(19) United States
(12) Patent Application Publication
(10) Pub. No.: US 2011/0166163 A1
(43) Pub. Date: Jul. 7, 2011

(54) SUBSTITUTED FUROPYRIMIDINES AND USE THEREOF

(75) Inventors: Thomas Lampe, Dusseldorf (DE); Raimund Kast, Wuppertal (DE); Mario Jeske, Solingen (DE); Friederike Stoll, Dusseldorf (DE); Joachim Schuhmacher, Wuppertal (DE); Eva-Maria Becker, Wuppertal (DE); Hartmut Beck, Koln (DE)

(73) Assignee: BAYER SCHERING PHARMA AKTIENGESELLSCHAFT, Berlin (DE)

(21) Appl. No.: 12/664,748

(22) PCT Filed: Jun. 3, 2008
(86) PCT No.: PCT/EP2008/004407

§ 371 Date: Mar. 28, 2011

(30) Foreign Application Priority Data
Jun. 16, 2007 (DE) 10 2007 027 799.9

(51) Int. Cl. A61K 31/519; C07D 491/048; A61P 9/12; A61P 9/00; A61P 7/02; A61P 9/10

(52) U.S. Cl. 514/260.1; 544/278

(57) ABSTRACT

The present application relates to novel substituted furopyrimidine derivatives, to processes for their preparation, to their use for the treatment and/or prophylaxis of diseases and to their use for preparing medicaments for the treatment and/or prophylaxis of diseases, especially for the treatment and/or prophylaxis of cardiovascular diseases.

The present application relates to novel substituted furopyrimidine derivatives, to processes for their preparation, to their use for the treatment and/or prophylaxis of diseases and to their use for preparing medicaments for the treatment and/or prophylaxis of diseases, especially for the treatment and/or prophylaxis of cardiovascular diseases.

Prostacyclin (PGI₂) belongs to the class of bioactive prostaglandins, which are derivatives of arachidonic acid. PGI₂ is the main product of arachidonic acid metabolism in endothelial cells and is a potent vasodilator and inhibitor of platelet aggregation. PGI₂ is the physiological antagonist of thromboxane A₂ (TxA₂), a strong vasoconstrictor and stimulator of platelet aggregation, and thus contributes to the maintenance of vascular homeostasis. A drop in PGI₂ levels is presumed to be partly responsible for the development of various cardiovascular diseases [Dusting, G. J. et al., Pharmac. Ther. 1990, 48: 323-344; Vane, J. et al., Eur. J. Vasc. Endovasc. Surg. 2003, 26: 571-578].

After release of arachidonic acid from phospholipids via phospholipases A₂, PGI₂ is synthesized by cyclooxygenases and then by PGI₂-synthase. PGI₂ is not stored, but is released immediately after synthesis, exerting its effects locally. PGI₂ is an unstable molecule, which is transformed rapidly (half-life approx. 3 minutes) and non-enzymatically, to an inactive metabolite, 6-keto-prostaglandin-F1 alpha [Dusting, G. J. et al., Pharmac. Ther. 1990, 48: 323-344].

The biological effects of PGI₂ occur through binding to a membrane-bound receptor, called the prostacyclin receptor or IP receptor [Narumiya, S. et al., Physiol. Rev. 1999, 79: 1193-1226]. The IP receptor is one of the G-protein-coupled receptors, which are characterized by seven transmembrane domains. In addition to the human IP receptor, prostacyclin receptors have also been cloned from rat and mouse [Vane, J. et al., Eur. J. Vasc. Endovasc. Surg. 2003, 26: 571-578]. In smooth muscle cells, activation of the IP receptor leads to stimulation of adenylate cyclase, which catalyzes the formation of cAMP from ATP. Increase in the intracellular cAMP concentration is responsible for prostacyclin-induced vasodilation and for inhibition of platelet aggregation. In addition to the vasoactive properties, anti-proliferative effects [Schroer, K. et al., Agents Actions Suppl. 1997, 48: 63-91; Kothapalli, D. et al., Mol. Pharmacol. 2003, 64: 249-258; Planchon, P. et al., Life Sci. 1995, 57: 1233-1240] and anti-arteriosclerotic effects [Rudic, R. D. et al., Circ. Res. 2005, 96: 1240-1247; Egan K. M. et al., Science 2004, 114: 784-794] have also been described for PGI₂. Furthermore, PGI₂ also inhibits the formation of metastases [Schneider, M. R. et al., Cancer Metastasis Rev. 1994, 13: 349-64]. It is unclear whether these effects are due to stimulation of cAMP formation or to IP receptor-mediated activation of other signal transduction pathways in the respective target cell [Wise, H. et al. TIPS 1996, 17: 17-21], such as the phosphoinositide cascade, and of potassium channels.

Although the effects of PGI₂ are on the whole of benefit therapeutically, clinical application of PGI₂ is severely restricted by its chemical and metabolic instability. PGI₂ analogs that are more stable, for example iloprost [Badesch, D. B. et al., J. Am. Coll. Cardiol. 2004, 43: 56S-61S] and treprostinil [Chattaraj, S. C., Curr. Opion. Invest. Drugs 2002, 3: 582-586] could be made available, but these compounds still have a very short time of action. Moreover, the substances can only be administered to the patient via complicated routes of administration, e.g. by continuous infusion, subcutaneously or via repeated inhalations. These routes of administration can also have additional side-effects, for example infections or pains at the site of injection. The use of beraprost, which to date is the only PGI₂ derivative available for oral administration to the patient [Barst, R. J. et al., J. Am. Coll. Cardiol. 2003, 41: 2119-2125], is once again limited by its short time of action.

The compounds described in the present application are, compared with PGI₂, chemically and metabolically stable, non-prostanoid activators of the IP receptor, which imitate the biological action of PGI₂ and thus can be used for the treatment of diseases, in particular of cardiovascular diseases.

DE 1 817 146, EP 1 018 514, EP 1 132 093, EP 1 724 268, WO 02/092603, WO 03/022852, WO 2005/092896, WO 2005/121149 and WO 2006/004658 describe various 4-oxy-, 4-thio- and/or 4-aminofuro[2,3-d]pyrimidine derivatives and their use for the treatment of diseases. WO 03/018589 discloses 4-aminofuropyrimidines as adenosine kinase inhibitors for the treatment of cardiovascular diseases. WO 2007/079861 and WO 2007/079862 claim substituted 5,6-diphenylfuropyrimidines as IP receptor agonists for the treatment of cardiovascular diseases.

In contrast to the compounds of the prior art, the compounds claimed in the context of the present application are distinguished by a 5,6-disubstituted furo[2,3-d]pyrimidine core structure which is attached via the 4-position at a certain spatial distance to a carboxylic acid or carboxylic acid-like functionality.

The present invention provides compounds of the general formula (I)

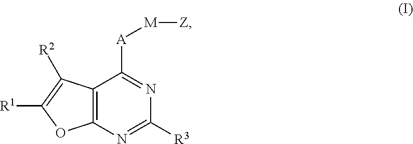

in which
A represents O or N—R⁴,
   where
   R⁴ represents hydrogen, (C₁-C₆)-alkyl, (C₃-C₇)-cycloalkyl or (C₄-C₇)-cycloalkenyl,
M represents a group of the formula

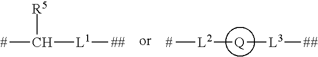

where
# represents the point of attachment to A,
## represents the point of attachment to Z,
R⁵ represents hydrogen or (C₁-C₄)-alkyl,
   where alkyl may be substituted by hydroxyl or amino,
L¹ represents (C₁-C₇)-alkanediyl, (C₂-C₇)-alkenediyl or a group of the formula *-L^{1A}-V-L^{1B}-**,
   where alkanediyl and alkenediyl may be mono- or disubstituted by fluorine,
   where
   * represents the point of attachment to —CHR⁵,
   ** represents the point of attachment to Z,
   L^{1A} represents (C₁-C₅)-alkanediyl,
      where alkanediyl may be substituted by 1 or 2 substituents independently of one another selected from the group consisting of (C₁-C₄)-alkyl and (C₁-C₄)-alkoxy,
   L^{1B} represents a bond or (C₁-C₃)-alkanediyl,
   where alkanediyl may be mono- or disubstituted by fluorine,
   and
   V represents O or N—R⁶,
      where
      R⁶ represents hydrogen, (C₁-C₆)-alkyl or (C₃-C₇)-cycloalkyl,
L² represents a bond or (C₁-C₄)-alkanediyl,
Q represents (C₃-C₇)-cycloalkyl, (C₄-C₇)-cycloalkenyl, 5- to 7-membered heterocyclyl, phenyl or 5- or 6-membered heteroaryl,
   where cycloalkyl, cycloalkenyl, heterocyclyl, phenyl and heteroaryl may each be substituted by 1 or 2 substituents independently of one another selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino,
      where alkyl may be substituted by a substituent selected from the group consisting of hydroxyl, (C₁-C₄)-alkoxy, amino, mono-(C₁-C₄)-alkylamino and di-(C₁-C₄)-alkylamino,
and
L³ represents (C₁-C₄)-alkanediyl or (C₂-C₄)-alkenediyl,
   where alkanediyl may be mono- or disubstituted by fluorine,
   and
   where a methylene group of the alkanediyl group may be replaced by O or N—R⁷,
      where
      R⁷ represents hydrogen, (C₁-C₆)-alkyl or (C₃-C₇)-cycloalkyl,
Z represents a group of the formula

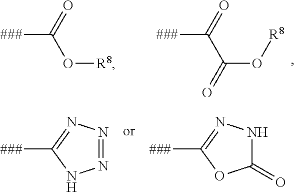

where
### represents the point of attachment to the group L¹ or L³,
and
R⁸ represents hydrogen or (C₁-C₄)-alkyl,
   and either
R¹ represents (C₃-C₇)-cycloalkyl, (C₄-C₇)-cycloalkenyl, 5- to 7-membered heterocyclyl or 5- or 6-membered heteroaryl,
   where cycloalkyl, cycloalkenyl, heterocyclyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
      where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
         and
R² represents (C₃-C₇)-cycloalkyl, (C₄-C₇)-cycloalkenyl, 5- to 7-membered heterocyclyl, phenyl or 5- or 6-membered heteroaryl,
   where cycloalkyl, cycloalkenyl, heterocyclyl, phenyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
      where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
   or
   two substituents attached to adjacent carbon atoms of a phenyl ring together form a group of the formula —O—CH₂—O—, —O—CHF—O—, —O—CF₂—O—, —O—CH₂—CH₂—O— or —O—CF₂—CF₂—O—,
      or
R¹ represents (C₃-C₇)-cycloalkyl, (C₄-C₇)-cycloalkenyl, 5- to 7-membered heterocyclyl, phenyl or 5- or 6-membered heteroaryl,
   where cycloalkyl, cycloalkenyl, heterocyclyl, phenyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
      where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
   or
   two substituents attached to adjacent carbon atoms of a phenyl ring together form a group of the formula —O—CH₂—O—, —O—CHF—O—, —O—CF₂—O—, —O—CH₂—CH₂—O— or —O—CF₂—CF₂—O—,
      and
R² represents (C₃-C₇)-cycloalkyl, (C₄-C₇)-cycloalkenyl, 5- to 7-membered heterocyclyl or 5- or 6-membered heteroaryl,
   where cycloalkyl, cycloalkenyl, heterocyclyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
      where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
         and
R³ represents hydrogen, (C₁-C₄)-alkyl or cyclopropyl,
   or their salts, solvates or solvates of the salts.

Compounds according to the invention are the compounds of the formula (I) and the salts, solvates and solvates of the salts thereof, the compounds of the formulae mentioned below encompassed by the formula (I) and the salts, solvates and solvates of the salts thereof, and also the compounds encompassed by the formula (I) and mentioned below as working examples, and the salts, solvates and solvates of the salts thereof, provided the compounds encompassed by formula (I) and mentioned below are not already salts, solvates and solvates of the salts.

The compounds of the invention may, depending on their structure, exist in stereoisomeric forms (enantiomers, diastereomers). The present invention therefore relates to the enantiomers or diastereomers and respective mixtures thereof. The stereoisomerically pure constituents can be isolated in a known manner from such mixtures of enantiomers and/or diastereomers.

If the compounds of the invention may occur in tautomeric forms, the present invention encompasses all tautomeric forms.

Salts which are preferred for the purposes of the present invention are physiologically acceptable salts of the compounds of the invention. Also encompassed are salts which are themselves unsuitable for pharmaceutical uses but can be used for example for isolating or purifying the compounds of the invention.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refers for the purposes of the invention to those forms of the compounds of the invention which form, in the solid or liquid state, a complex by coordination with solvent molecules. Hydrates are a specific form of solvates in which the coordination takes place with water. Hydrates are preferred solvates in the context of the present invention.

The present invention additionally encompasses prodrugs of the compounds of the invention. The term “prodrugs” encompasses compounds which themselves may be biologically active or inactive, but are converted during their residence time in the body into compounds of the invention (for example by metabolism or hydrolysis).

In particular, for the compounds of the formula (I) in which

Z represents a group of the formula

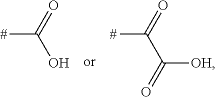

the present invention also includes hydrolyzable ester derivatives of these compounds. These are to be understood as meaning esters which can be hydrolyzed to the free carboxylic acids, as the compounds that are mainly active biologically, in physiological media, under the conditions of the biological tests described later and in particular in vivo by enzymatic or chemical routes. (C₁-C₄)-alkyl esters, in which the alkyl group can be straight-chain or branched, are preferred as such esters. Particular preference is given to methyl or ethyl esters (see also the corresponding definitions of the radical R⁸).

In the context of the present invention, the substituents have the following meaning, unless specified otherwise:

Alkyl stands in the context of the invention for a straight-chain or branched alkyl radical having 1 to 6 carbon atoms. Preference is given to a straight-chain or branched alkyl radical having 1 to 4 carbon atoms. The following may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

Alkenyl stands in the context of the invention for a straight-chain or branched alkenyl radical having 2 to 6 carbon atoms and one or two double bonds. Preference is given to a straight-chain or branched alkenyl radical having 2 to 5 carbon atoms and one double bond. The following may be mentioned by way of example and by way of preference: vinyl, allyl, isopropenyl and n-but-2-en-1-yl.

Alkynyl stands in the context of the invention for a straight-chain or branched alkynyl radical having 2 to 4 carbon atoms and a triple bond. The following may be mentioned by way of example and by way of preference: ethynyl, n-prop-1-yn-1-yl, n-prop-2-yn-1-yl, n-but-2-yn-1-yl and n-but-3-yn-1-yl.

Alkanediyl stands in the context of the invention for a straight-chain or branched divalent alkyl radical having 1 to 7 carbon atoms. The following may be mentioned by way of example and by way of preference: methylene, 1,2-ethylene, ethane-1,1-diyl, 1,3-propylene, propane-1,1-diyl, propane-1,2-diyl, propane-2,2-diyl, 1,4-butylene, butane-1,2-diyl, butane-1,3-diyl and butane-2,3-diyl.

Alkenediyl stands in the context of the invention for a straight-chain or branched divalent alkenyl radical having 2 to 7 carbon atoms and up to 2 double bonds. The following may be mentioned by way of example and by way of preference: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl and buta-1,3-diene-1,4-diyl.

Alkoxy stands in the context of the invention for a straight-chain or branched alkoxy radical having 1 to 6 carbon atoms. Preference is given to a straight-chain or branched alkoxy radical having 1 to 4 carbon atoms. The following may be mentioned by way of example and by way of preference: methoxy, ethoxy, n-propoxy, isopropoxy, n-butoxy, tert-butoxy, n-pentoxy and n-hexoxy.

Alkylthio stands in the context of the invention for a straight-chain or branched alkylthio radical having 1 to 6 carbon atoms. Preference is given to a straight-chain or branched alkylthio radical having 1 to 4 carbon atoms. The following may be mentioned by way of example and by way of preference: methylthio, ethylthio, n-propylthio, isopropylthio, n-butylthio, tert-butylthio, n-pentylthio and n-hexylthio.

Alkylcarbonyl stands in the context of the invention for a straight-chain or branched alkyl radical having 1 to 6 carbon atoms and a carbonyl group attached in position 1. The following may be mentioned by way of example and by way of preference: methylcarbonyl, ethylcarbonyl, n-propylcarbonyl, isopropylcarbonyl, n-butylcarbonyl, isobutylcarbonyl and tert-butylcarbonyl.

Monoalkylamino stands in the context of the invention for an amino group having a straight-chain or branched alkyl substituent having 1 to 6 carbon atoms. The following may be mentioned by way of example and by way of preference: methylamino, ethylamino, n-propylamino, isopropylamino and tert-butylamino.

Dialkylamino stands in the context of the invention for an amino group having two identical or different straight-chain or branched alkyl substituents having 1 to 6 carbon atoms each. The following may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Alkylcarbonylamino stands in the context of the invention for an amino group which is attached via a carbonyl group to a straight-chain or branched alkyl substituent having 1 to 6 carbon atoms. The following may be mentioned by way of example and by way of preference: methylcarbonylamino, ethylcarbonylamino, n-propylcarbonylamino, isopropylcarbonylamino, n-butylcarbonylamino, isobutylcarbonylamino and tert-butylcarbonylamino.

Cycloalkyl stands in the context of the invention for a monocyclic saturated cycloalkyl group having 3 to 7 carbon atoms. The following may be mentioned by way of example and by way of preference: cyclopropyl, cyclobutyl, cyclopentyl, cyclohexyl and cycloheptyl.

Cycloalkenyl stands in the context of the invention for a monocyclic cycloalkyl group having 4 to 7 carbon atoms and a double bond. The following may be mentioned by way of example and by way of preference: cyclobutenyl, cyclopentenyl, cyclohexenyl and cycloheptenyl.

Heterocyclyl stands in the context of the invention for a saturated monocyclic heterocyclic radical having 5 to 7 ring atoms and up to 3, preferably up to 2, heteroatoms and/or heterogroups from the series N, O, S, SO, SO₂, where a nitrogen atom may also form an N-oxide. Preference is given to 5- or 6-membered saturated heterocyclyl radicals having one or two ring heteroatoms from the series N and O. The following may be mentioned by way of example and by way of preference: pyrrolidinyl, pyrrolinyl, pyrazolidinyl, tetrahydrofuranyl, piperidinyl, piperazinyl, tetrahydropyranyl, morpholinyl, hexahydroazepinyl and hexahydro-1,4-diazepinyl.

Heteroaryl stands in the context of the invention for an aromatic heterocycle (heteroaromatic) having 5 or 6 ring atoms and up to 3 heteroatoms from the series N, O and S, where a nitrogen atom may also form an N-oxide. The following may be mentioned by way of example and by way of preference: furyl, pyrrolyl, thienyl, pyrazolyl, imidazolyl, thiazolyl, oxazolyl, oxadiazolyl, isoxazolyl, isothiazolyl, pyridyl, pyrimidinyl, pyridazinyl and pyrazinyl.

Halogen stands in the context of the invention for fluorine, chlorine, bromine and iodine, preferably for chlorine or fluorine.

If radicals in the compounds according to the invention are substituted, the radicals, unless specified otherwise, may be mono- or polysubstituted. In the context of the present invention, for all radicals that occur more than once, their meanings are independent of one another. Substitution by one, two or three identical or different substituents is preferred. Very particular preference is given to substitution by one substituent.

In the context of the present invention, preference is given to compounds of the formula (I) in which
A represents O or N—R⁴,
   where
   R⁴ represents hydrogen, (C₁-C₄)-alkyl or cyclopropyl,
M represents a group of the formula

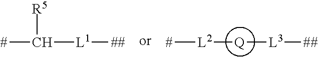

where
# represents the point of attachment to A,
## represents the point of attachment to Z,
R⁵ represents hydrogen or (C₁-C₃)-alkyl,
   where alkyl may be substituted by hydroxyl or amino,
L¹ represents (C₃-C₇)-alkanediyl, (C₃-C₇)-alkenediyl or a group of the formula *-L^{1A}-V-L^{1B}-**,
   where alkanediyl and alkenediyl may be mono- or disubstituted by fluorine,
   where
   * represents the point of attachment to —CHR⁵,
   ** represents the point of attachment to Z,
   L^{1A} represents (C₁-C₃)-alkanediyl,
      where alkanediyl may be substituted by 1 or 2 substituents independently of one another selected from the group consisting of methyl and ethyl,
   L^{1B} represents (C₁-C₃)-alkanediyl,
   where alkanediyl may be mono- or disubstituted by fluorine,
   and
   V represents O or N—R⁶,
      where
      R⁶ represents hydrogen, (C₁-C₃)-alkyl or cyclopropyl,
L² represents a bond or (C₁-C₃)-alkanediyl,
Q represents (C₃-C₆)-cycloalkyl, (C₄-C₆)-cycloalkenyl, 5- or 6-membered heterocyclyl or phenyl,
   where cycloalkyl, cycloalkenyl, heterocyclyl and phenyl may each be substituted by 1 or 2 substituents independently of one another selected from the group consisting of fluorine, chlorine, (C₁-C₃)-alkyl, trifluoromethyl, hydroxyl, methoxy, ethoxy, trifluoromethoxy, amino, methylamino, dimethylamino, ethylamino and diethylamino,
and
L³ represents (C₁-C₃)-alkanediyl, (C₂-C₃)-alkenediyl or a group of the formula -W—CR⁹R¹⁰-, -W—CH₂—CR⁹R¹⁰- or -CH₂—W—CR⁹R¹⁰-,
   where alkanediyl may be mono- or disubstituted by fluorine,
   where
   represents the point of attachment to ring Q,
   represents the point of attachment to the group Z,
   W represents O or N—R¹¹,
      where
      R¹¹ represents hydrogen, (C₁-C₃)-alkyl or cyclopropyl,
   R⁹ represents hydrogen or fluorine,
   and
   R¹⁰ represents hydrogen or fluorine,
Z represents a group of the formula

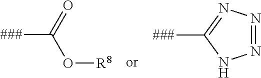

where
### represents the point of attachment to the group L¹ or L³,
and
R⁸ represents hydrogen, methyl or ethyl,
   and either
R¹ represents (C₄-C₆)-cycloalkyl, (C₄-C₆)-cycloalkenyl, 5- or 6-membered heterocyclyl or 5- or 6-membered heteroaryl,
   where cycloalkyl, cycloalkenyl, heterocyclyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
      and
R² represents phenyl or 5- or 6-membered heteroaryl,
   where phenyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
   or
   two substituents attached to adjacent carbon atoms of a phenyl ring together form a group of the formula —O—CH₂—O—, —O—CHF—O— or —O—CF₂—O—,
      or
R¹ represents phenyl or 5- or 6-membered heteroaryl,
   where phenyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
   or
   two substituents attached to adjacent carbon atoms of a phenyl ring together form a group of the formula —O—CH₂—O—, —O—CHF—O— or —O—CF₂—O—,
      and
R² represents (C₄-C₆)-cycloalkyl, (C₄-C₆)-cycloalkenyl, 5- or 6-membered heterocyclyl or 5- or 6-membered heteroaryl,
   where cycloalkyl, cycloalkenyl, heterocyclyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
      and
R³ represents hydrogen or (C₁-C₃)-alkyl,
   or their salts, solvates or solvates of the salts.

In the context of the present invention, particular preference is given to compounds of the formula (I) in which
A represents O or NH,
M represents a group of the formula

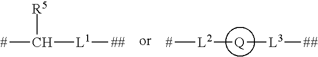

where
# represents the point of attachment to A,
## represents the point of attachment to Z,
R⁵ represents hydrogen, methyl or ethyl,
L¹ represents (C₃-C₇)-alkanediyl, (C₃-C₇)-alkenediyl or a group of the formula *-L^{1A}-V-L^{1B}-**,
   where
   * represents the point of attachment to —CHR⁵,
   ** represents the point of attachment to Z,
   L^{1A} represents (C₁-C₃)-alkanediyl,
      where alkanediyl may be substituted by 1 or 2 substituents independently of one another selected from the group consisting of methyl and ethyl,
   L^{1B} represents (C₁-C₃)-alkanediyl,
   and
   V represents O or N—CH₃,
L² represents a bond, methylene, ethane-1,1-diyl or ethane-1,2-diyl,
Q represents cyclobutyl, cyclopentyl, cyclohexyl, piperidinyl or phenyl,
   where cyclobutyl, cyclopentyl, cyclohexyl, piperidinyl and phenyl may each be substituted by 1 or 2 substituents independently of one another selected from the group consisting of fluorine, methyl, ethyl, trifluoromethyl, hydroxyl, methoxy and ethoxy,
and
L³ represents (C₁-C₃)-alkanediyl or a group of the formula -W—CH₂ or -W—CH₂—CH₂-,
   where
   represents the point of attachment to ring Q,
   represents the point of attachment to the group Z,
   W represents O or N—R¹¹,
      where
      R¹¹ represents hydrogen or (C₁-C₃)-alkyl,
Z represents a group of the formula

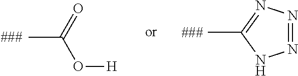

where
### represents the point of attachment to the group L¹ or L³,
   and either
R¹ represents (C₄-C₆)-cycloalkyl, (C₄-C₆)-cycloalkenyl or 5- or 6-membered heteroaryl,
   where cycloalkyl, cycloalkenyl and heteroaryl groups may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      and
R² represents phenyl,
   where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      or
R¹ represents phenyl,
   where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      and
R² represents (C₄-C₆)-cycloalkyl, (C₄-C₆)-cycloalkenyl or 5- or 6-membered heteroaryl,
   where cycloalkyl, cycloalkenyl and heteroaryl may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      and
R³ represents hydrogen or methyl,
   or their salts, solvates or solvates of the salts.

In the context of the present invention, particular preference is also given to compounds of the formula (I) in which
A represents O or NH,
M represents a group of the formula

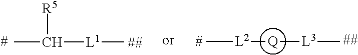

where
# represents the point of attachment to A,
## represents the point of attachment to Z,
R⁵ represents hydrogen, methyl or ethyl,
L¹ represents (C₃-C₇)-alkanediyl, (C₃-C₇)-alkenediyl or a group of the formula *-L^{1A}-V-L^{1B}-**,
   where
   * represents the point of attachment to —CHR⁵,
   ** represents the point of attachment to Z,
   L^{1A} represents (C₁-C₃)-alkanediyl,
   L^{1B} represents (C₁-C₃)-alkanediyl,
   and
   V represents O or N—CH₃,
L² represents a bond, methylene, ethane-1,1-diyl or ethane-1,2-diyl,
Q represents cyclobutyl, cyclopentyl, cyclohexyl, cyclopentenyl, pyrrolodinyl, piperidinyl, tetrahydrofuranyl, tetrahydropyranyl, morpholinyl or phenyl,
   where cyclobutyl, cyclopentyl, cyclohexyl, cyclopentenyl, pyrrolodinyl, piperidinyl, tetrahydrofuranyl, tetrahydropyranyl, morpholinyl and phenyl may each be substituted by 1 or 2 substituents independently of one another selected from the group consisting of fluorine, methyl, ethyl, trifluoromethyl, hydroxyl, methoxy and ethoxy,
and
L³ represents (C₁-C₃)-alkanediyl or a group of the formula -W—CH₂ or -W—CH₂—CH₂-,
   where
   represents the point of attachment to ring Q,
   represents the point of attachment to the group Z,
   W represents O or N—R¹¹,
      where
      R¹¹ represents hydrogen or (C₁-C₃)-alkyl,
Z represents a group of the formula

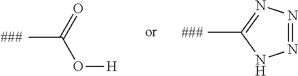

where
### represents the point of attachment to the group L¹ or L³, and either
R¹ represents (C₄-C₆)-cycloalkyl, (C₄-C₆)-cycloalkenyl or 5- or 6-membered heteroaryl,
   where cycloalkyl, cycloalkenyl and heteroaryl may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      and
R² represents phenyl,
   where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      or
R¹ represents phenyl,
   where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      and
R² represents (C₄-C₆)-cycloalkyl, (C₄-C₆)-cycloalkenyl or 5- or 6-membered heteroaryl,
   where cycloalkyl, cycloalkenyl and heteroaryl may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
      and
R³ represents hydrogen or methyl,
   or their salts, solvates or solvates of the salts.

In the context of the present invention, very particular preference is given to compounds of the formula (I) in which
A represents O or NH,
M represents a group of the formula

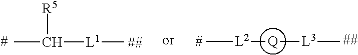

where
# represents the point of attachment to A,
## represents the point of attachment to Z,
R⁵ represents hydrogen or methyl,
L¹ represents butane-1,4-diyl or pentane-1,5-diyl,
L² represents a bond or methylene,
Q represents cyclopentyl, cyclohexyl or phenyl,
and
L³ represents methylene, ethane-1,2-diyl, propane-1,3-diyl or a group of the formula -O—CH₂- or -O—CH₂—CH₂-,
   where
   represents the point of attachment to ring Q,
   represents the point of attachment to the group Z,
Z represents a group of the formula

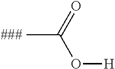

where
### represents the point of attachment to the group L¹ or L³,
   and either
R¹ represents cyclopent-1-en-1-yl, cyclohexen-1-en-1-yl, thienyl or pyridyl,
   where cyclopent-1-en-1-yl and cyclohexen-1-en-1-yl may each be monosubstituted by fluorine,
   and
   where thienyl and pyridyl may each be substituted by a substituent selected from the group consisting of fluorine, chlorine, methyl and trifluoromethyl,
      and
R² represents phenyl,
   where phenyl may be substituted by a substituent from the group consisting of methyl, ethyl, vinyl, trifluoromethyl, methoxy, ethoxy and trifluoromethoxy,
      or
R¹ represents phenyl,
   where phenyl may be substituted by a substituent selected from the group consisting of fluorine, chlorine, methyl, trifluoromethyl, methoxy and trifluoromethoxy,
      and
R² represents cyclopent-1-en-1-yl, cyclohexen-1-en-1-yl or pyridyl, where cyclopent-1-en-1-yl and cyclohexen-1-en-1-yl may each be substituted by a substituent selected from the group consisting of methyl, ethyl, trifluoromethyl and methoxy,
   and
   where pyridyl may be substituted by a substituent selected from the group consisting of methyl, ethyl, vinyl, trifluoromethyl, methoxy, ethoxy and trifluoromethoxy,
      and
R³ represents hydrogen,
   or their salts, solvates or solvates of the salts.

In the context of the present invention, preference is also given to compounds of the formula (I) in which

either
R¹ represents cyclopent-1-en-1-yl, cyclohexen-1-en-1-yl, thienyl or pyridyl,
   where cyclopent-1-en-1-yl and cyclohexen-1-en-1-yl may each be monosubstituted by fluorine,
   and
   where thienyl and pyridyl may each be substituted by a substituent selected from the group consisting of fluorine, chlorine, methyl and trifluoromethyl,
      and
R² represents phenyl,
   where phenyl may be substituted by a substituent from the group consisting of methyl, ethyl, vinyl, trifluoromethyl, methoxy, ethoxy and trifluoromethoxy,
      or
R¹ represents phenyl,
   where phenyl may be substituted by a substituent selected from the group consisting of fluorine, chlorine, methyl, trifluoromethyl, methoxy and trifluoromethoxy,
      and
R² represents cyclopent-1-en-1-yl, cyclohexen-1-en-1-yl or pyridyl,
   where cyclopent-1-en-1-yl and cyclohexen-1-en-1-yl may each be substituted by a substituent selected from the group consisting of methyl, ethyl, trifluoromethyl and methoxy,
   and
   where pyridyl may be substituted by a substituent selected from the group consisting of methyl, ethyl, vinyl, trifluoromethyl, methoxy, ethoxy and trifluoromethoxy.

In the context of the present invention, preference is also given to compounds of the formula (I) in which
M represents a group of the formula

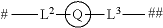

where
# represents the point of attachment to A,
## represents the point of attachment to Z,
L² represents a bond or methylene,
Q represents cyclopentyl, cyclohexyl or phenyl,
and
L³ represents methylene, ethane-1,2-diyl, propane-1,3-diyl or a group of the formula -O—CH₂- or -O—CH₂—CH₂-,
   where
   represents the point of attachment to ring Q,
   represents the point of attachment to the group Z.

In the context of the present invention, preference is also given to compounds of the formula (I) in which
M represents a group of the formula

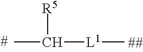

where
# represents the point of attachment to A,
## represents the point of attachment to Z,
R⁵ represents hydrogen, methyl or ethyl,
L¹ represents (C₃-C₇)-alkanediyl, (C₃-C₇)-alkenediyl or a group of the formula *-L^{1A}-V-L^{1B}-**,
   where
   * represents the point of attachment to —CHR⁵,
   ** represents the point of attachment to Z,
   L^{1A} represents (C₁-C₃)-alkanediyl,
      where alkanediyl may be substituted by 1 or 2 substituents independently of one another selected from the group consisting of methyl and ethyl,
   L^{1B} represents (C₁-C₃)-alkanediyl,
   and
   V represents O or N—CH₃.

The individual definitions of radicals given in the respective combinations and preferred combinations of radicals are, independently of the given combination of radicals in question, also optionally replaced by radical definitions of other combinations.

Very particular preference is given to combinations of two or more of the preferred ranges mentioned above.

The invention furthermore provides a process for preparing the compounds of the formula (I) according to the invention in which Z represents —COOH, characterized in that either
[A] compounds of the formula (II-A)

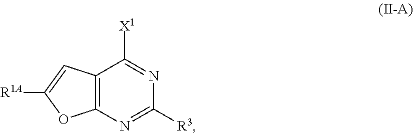

in which R³ has the meaning given above
and
R^{1A} represents (C₄-C₇)-cycloalkenyl, 5- or 6-membered heteroaryl or phenyl,
   where the cycloalkenyl, heteroaryl and phenyl groups mentioned may be substituted within the scope of the meanings mentioned above,
and
X¹ represents a leaving group, such as, for example, halogen, in particular chlorine,
are reacted in the presence of a base, if appropriate in an inert solvent, with a compound of the formula (III)

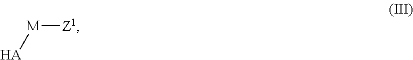

in which A and M each have the meanings given above
and
Z¹ represents cyano or a group of the formula COOR^{8A},
   where
   R^{8A}, represents (C₁-C₄)-alkyl,
to give compounds of the formula (IV-A)

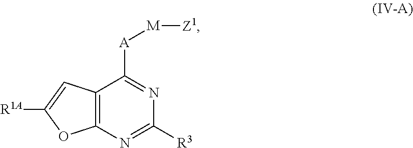

in which A, M, Z¹, R^{1A} and R³ each have the meanings given above,
then brominated in an inert solvent, for example with N-bromosuccinimide, to give compounds of the formula (V-A)

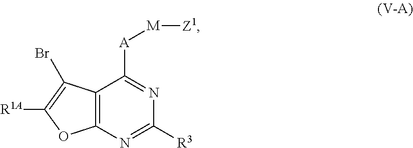

in which A, M, Z¹, R^{1A} and R³ have the meanings given above,
and these are subsequently coupled in an inert solvent in the presence of a base and a suitable palladium catalyst with a compound of the formula (VI-A)

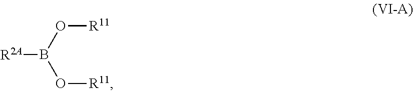

in which
R^{2A} represents (C₄-C₇)-cycloalkenyl or 5- or 6-membered heteroaryl,
   where the cycloalkenyl and heteroaryl groups mentioned may be substituted within the scope of the meanings mentioned above,
and
R¹¹ represents hydrogen or the two radicals R¹¹ together form a —C(CH₃)₂—C(CH₃)₂-bridge,
to give compounds of the formula (VII-A)

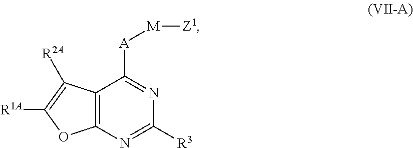

in which A, M, Z¹, R^{1A}, R^{2A} and R³ have the scope of the meanings given above,
   or
[B] compounds of the formula (II-B)

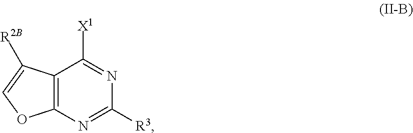

in which X¹ and R³ have the meanings given above
and
R^{2B} represents (C₄-C₇)-cycloalkenyl, 5- or 6-membered heteroaryl or phenyl,
   where the cycloalkenyl, heteroaryl and phenyl groups mentioned may be substituted within the scope of the meanings mentioned above,
are reacted in the presence of a base, if appropriate in an inert solvent, with a compound of the formula (III) to give compounds of the formula (IV-B)

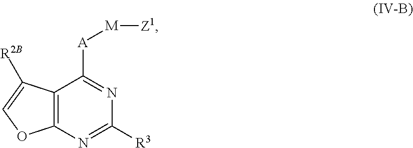

in which A, M, Z¹, R^{2B} and R³ each have the meanings given above,
then brominated in an inert solvent, for example with N-bromosuccinimide, to give compounds of the formula (V-B)

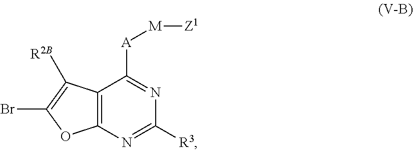

in which A, M, Z¹, R^{2B} and R³ have the meanings given above,
and these are subsequently coupled in an inert solvent in the presence of a base and a suitable palladium catalyst with a compound of the formula (VI-B)

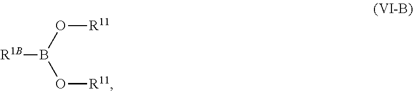

in which R¹¹ has the scope of the meanings indicated above
and
R^{1B} represents (C₄-C₇)-cycloalkenyl or 5- or 6-membered heteroaryl,
   where the cycloalkenyl and heteroaryl groups mentioned may be substituted within the scope of the meanings mentioned above,
to give compounds of the formula (VII-B)

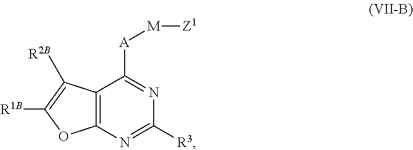

in which A, M, Z¹, R^{1B}, R^{2B} and R³ have the scope of the meanings given above,
   or
[C] compounds of the formula (V-A) are coupled in an inert solvent in the presence of a suitable palladium catalyst with compounds of the formula (VI-C)

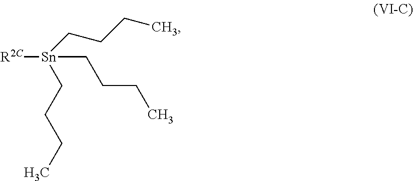

in which
R^{2c} represents 5- or 6-membered heteroaryl,
   where heteroaryl may be substituted within the scope of the meanings mentioned above,
to give compounds of the formula (VII-C)

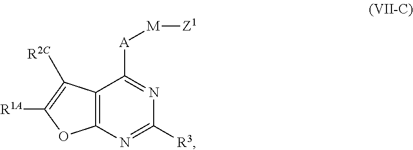

in which A, M, Z¹, R^{1A}, R^{2C} and R³ have the scope of the meanings given above,
   or
[D] compounds of the formula (V-B) are coupled in an inert solvent in the presence of a suitable palladium catalyst with a compound of the formula (VI-D)

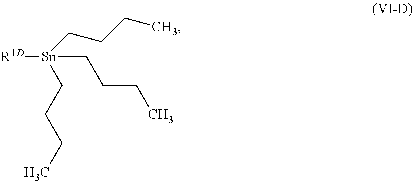

in which
R^{1D} represents 5- or 6-membered heteroaryl,
   where heteroaryl may be substituted within the scope of the meanings mentioned above,
to give compounds of the formula (VII-D)

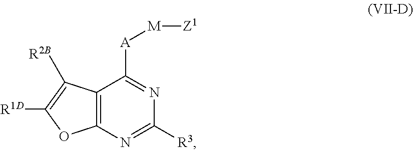

in which A, M, Z¹, R^{1D}, R^{2B} and R³ have the scope of the meanings given above,
   or
[E] compounds of the formula (V-A) are coupled in an inert solvent, if appropriate in the presence of a base and a suitable palladium catalyst, with 4,4,4′,4′,5,5,5′,5′-octamethyl-2,2′-bi-1,3,2-dioxoborolane (bis(pinacolato)-diboron) to give compounds of the formula (V-E)

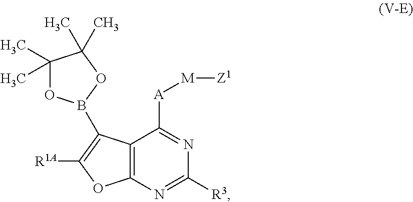

in which A, M, Z¹, R^{1A} and R³ have the scope of the meanings given above, and then coupled with compounds of the formula (VI-E)

R^{2C}—X² (VI-E)
in which R^{2C} has the scope of the meanings indicated above
and
X² represents a leaving group, such as, for example, halogen or trifluoromethanesulfonyloxy, in particular bromine or trifluoromethanesulfonyloxy,
in an inert solvent, if appropriate in the presence of a base and a suitable palladium catalyst, to give compounds of the formula (VII-C),
   and the respective compounds of the formulae (VII-A), (VII-B), (VII-C) and (VII-D) obtained are then converted by hydrolysis of the cyano or ester group Z¹ into the carboxylic acids of the formula (I-1)

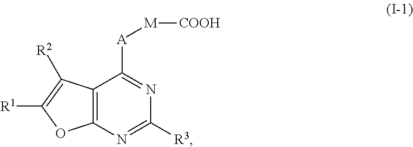

in which A, M, R¹, R² and R³ have the meanings given above,
and these are, if appropriate, reacted with the appropriate (i) solvents and/or (ii) bases or acids to give their solvates, salts and/or solvates of the salts.

Inert solvents for process steps (II-A)+(III)→(IV-A) and (II-B)+(III)→(IV-B) are, for example, ethers, such as diethyl ether, methyl tert-butyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethane, tetrachloroethane, trichloroethylene, chlorobenzene or chlorotoluene, or other solvents, such as dimethylformamide (DMF), dimethyl sulfoxide (DMSO), N,N′-dimethylpropyleneurea (DMPU), N-methylpyrrolidone (NMP) or acetonitrile. It is also possible to use mixtures of the solvents mentioned. Preference is given to using tetrahydrofuran, dimethylformamide, dimethyl sulfoxide or mixtures of these solvents.

However, if appropriate, the process steps (II-A)+(III)→(IV-A) and (II-B)+(III)→(IV-B) can also be carried out in the absence of a solvent.

Suitable bases for process steps (II-A)+(III)→(IV-A) and (II-B)+(III)→(IV-B) are customary inorganic or organic bases. These preferably include alkali metal hydroxides, such as, for example, lithium hydroxide, sodium hydroxide, or potassium hydroxide, alkali metal or alkaline earth metal carbonates, such as lithium carbonate, sodium carbonate, potassium carbonate, calcium carbonate or cesium carbonate, alkali metal alkoxides, such as sodium tert-butoxide or potassium tert-butoxide, alkali metal hydrides, such as sodium hydride or potassium hydride, amides, such as lithium bis(trimethylsilyl)amide or potassium bis(trimethylsilyl)amide or lithium diisopropylamide, organic metallic compounds, such as butyllithium or phenyllithium, or organic amines, such as triethylamine, N-methylmorpholine, N-methylpiperidine, N,N-diisopropyl-ethylamine or pyridine.

In the case of the reaction with alcohol derivatives [A in (III)=O], phosphazene bases (so-called “Schwesinger bases”), such as, for example, P2-t-Bu or P4-t-Bu are likewise expedient [cf., for example, R. Schwesinger, H. Schlemper, Angew. Chem. Int. Ed. Engl. 26, 1167 (1987); T. Pietzonka, D. Seebach, Chem. Ber. 124, 1837 (1991)].

In the reaction with amine derivatives [A in (III)=N], the base used is preferably a tertiary amine, such as, in particular, N,N-diisopropylethylamine. However, if appropriate, these reactions can—if an excess of the amine component (III) is used—also be carried out without the addition of an auxiliary base. In the reaction with alcohol derivatives [A in (III)=O], preference is given to potassium carbonate or cesium carbonate or the phosphazene bases P2-t-Bu and P4-t-Bu.

If appropriate, the process steps (II-A)+(III)→(IV-A) and (II-B)+(III)→(IV-B) can advantageously be carried out with addition of a crown ether.

In one process variant, the reactions (II-A)+(III)→(IV-A) and (II-B)+(III)→(IV-B) can also be carried out in a two-phase mixture consisting of an aqueous alkali metal hydroxide solution as base and one of the hydrocarbons or halogenated hydrocarbons mentioned above as further solvent, using a phase-transfer catalyst, such as tetrabutyl-ammonium hydrogen sulfate or tetrabutylammonium bromide.

The process steps (II-A)+(III)→(IV-A) and (II-B)+(III)→(IV-B) are, in the reaction with amine derivatives [A in (III)=N], generally carried out in a temperature range of from +50° C. to +150° C. In the reaction with alcohol derivatives [A in (III)=O], the reactions are generally carried out in a temperature range of from −20° C. to +120° C., preferably at from 0° C. to +60° C.

The bromination in process steps (IV-A)→(V-A) and (IV-B)→(V-B) is preferably carried out in a halogenated hydrocarbon as solvent, in particular in carbon tetrachloride, in a temperature range of from +50° C. to +100° C. Suitable brominating agents are elemental bromine and also, in particular, N-bromosuccinimide (NBS), if appropriate with addition of α,α′-azobis(isobutyronitrile) (AIBN) as initiator.

Inert solvents for process steps (V-A)+(VI-A)→(VII-A), (V-B)+(VI-B)→(VII-B), (V-E)+(VI-E)→(VII-C) and (V-A)+(bis(pinacolato)diboron)→(VI-E) are, for example, alcohols, such as methanol, ethanol, n-propanol, isopropanol, n-butanol or tert-butanol, ethers, such as diethyl ether, dioxane, tetrahydrofuran, glycol dimethyl ether or di-ethylene glycol dimethyl ether, hydrocarbons, such as benzene, xylene, toluene, hexane, cyclohexane or mineral oil fractions, or other solvents, such as dimethyl-formamide, dimethyl sulfoxide, N,N′-dimethylpropyleneurea (DMPU), N-methylpyrrolidone (NMP), pyridine, acetonitrile or else water. It is also possible to use mixtures of the solvents mentioned. Preference is given to a mixture of dimethyl sulfoxide and water.

Suitable bases for the process steps (V-A)+(VI-A)→(VII-A), (V-B)+(VI-B)→(VII-B), (V-E)+(VI-E)→(VII-C) and (V-A)+(bis(pinacolato)-diboron)→(V-E) are customary inorganic bases. These include in particular alkali metal hydroxides, such as, for example, lithium hydroxide, sodium hydroxide or potassium hydroxide, alkali metal bicarbonates, such as sodium bicarbonate or potassium bicarbonate, alkali metal carbonate and alkaline earth metal carbonates, such as lithium carbonate, sodium carbonate, potassium carbonate, calcium carbonate or cesium carbonate, or alkali metal hydrogenphosphates, such as disodium hydrogenphosphate or dipotassium hydrogenphosphate. Preference is given to using sodium carbonate or potassium carbonate.

Suitable palladium catalysts for the process steps (V-A)+(VI-A)→(VII-A), (V-B)+(VI-B)→(VII-B), (V-E)+(VI-E)→(VII-C) and (V-A)+(bis(pinacolato)-diboron)→(V-E) [“Suzuki coupling”] are, for example, palladium on carbon, palladium(II) acetate, tetrakis(triphenylphosphine)palladium(0), bis(triphenylphosphine)palladium(II) chloride, bis-(acetonitrile)palladium(II) chloride and [1,1′-bis(diphenylphosphino)ferrocene]-dichloropalladium(II)/dichloromethane complex [c.f., for example, J. Hassan et al., Chem. Rev. 102, 1359-1469 (2002)].

The reactions (V-A)+(VI-A)→(VII-A), (V-B)+(VI-B)→(VII-B), (V-E)+(VI-E)→(VII-C) and (V-A)+(bis(pinacolato)diboron)→(V-E) are generally carried out in a temperature range of from +20° C. to +150° C., preferably at from +50° C. to +100° C.

Inert solvents for process steps (V-A)+(VI-C)→(VII-C) and (V-B)+(VI-D)→(VII-D) are, for example, ethers, such as dioxane, tetrahydrofuran, glycol dimethyl ether or di-ethylene glycol dimethyl ether, hydrocarbons, such as benzene, xylene, toluene, hexane, cyclohexane or mineral oil fractions, or other solvents, such as dimethylformamide, dimethyl sulfoxide, N,N′-dimethylpropyleneurea (DMPU), N-methyl-pyrrolidone (NMP), pyridine, acetonitrile or else water. It is also possible to use mixtures of the solvents mentioned. Preference is given to using toluene.

Suitable palladium catalysts for process steps (V-A)+(VI-C)→(VII-C) and (V-B)+(VI-D)→(VII-D) [“Stille coupling”] are palladium(0) or palladium(II) compounds, in particular bis(dibenzylideneacetone)palladium(0), tetrakis(triphenylphosphine)palladium(0), palladium(II) acetate, bis(triphenylphosphine)palladium(II) chloride [see also: V. Farina, V. Krishnamurthy, W. J. Scott in: The Stille Reaction, 1998, J. Wiley and Sons, New York]. The reactions (V-A)+(VI-C)→(VII-C) and (V-B)+(VI-D)→(VII-D) are generally carried out in a temperature range of from +60° C. to +150° C., preferably at from +100° C. to +130° C.

The hydrolysis of the cyano or ester group Z¹ of the compounds (VII-A), (VII-B), (VII-C) and (VII-D) to give compounds of the formula (I-1) is carried out by customary methods by treating the esters or nitriles in inert solvents with acids or bases, where in the latter case the salts initially formed are converted by treatment with acid into the free carboxylic acids. In the case of the tert-butyl esters, the ester cleavage is preferably carried out using acids.

Suitable inert solvents for these reactions are water or the organic solvents customary for ester cleavage. These preferably include alcohols, such as methanol, ethanol, n-propanol, isopropanol, n-butanol or tert-butanol, or ethers, such as diethyl ether, tetra-hydrofuran, dioxane or glycol dimethyl ether, or other solvents, such as acetone, dichloromethane, dimethylformamide or dimethyl sulfoxide. It is also possible to use mixtures of the solvents mentioned. In the case of a basic ester hydrolysis, preference is given to using mixtures of water with dioxane, tetrahydrofuran, methanol and/or ethanol, and for nitrile hydrolysis, preference is given to using water and/or n-propanol. In the case of the reaction with trifluoroacetic acid, preference is given to using dichloromethane, and in the case of the reaction with hydrogen chloride, preference is given to using tetrahydrofuran, diethyl ether, dioxane or water.

Suitable bases are the customary inorganic bases. These preferably include alkali metal hydroxides or alkaline earth metal hydroxides, such as, for example, sodium hydroxide, lithium hydroxide, potassium hydroxide or barium hydroxide, or alkali metal carbonates or alkaline earth metal carbonates, such as sodium carbonate, potassium carbonate or calcium carbonate. Particular preference is given to sodium hydroxide or lithium hydroxide.

Acids suitable for the ester cleavage are, in general, sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, if appropriate with added water. Preference is given to hydrogen chloride or trifluoroacetic acid in the case of the tert-butyl esters and to hydrochloric acid in the case of the methyl esters.

The ester cleavage is generally carried out in a temperature range of from 0° C. to +100° C., preferably at from +0° C. to +50° C.

The reactions mentioned can be carried out at atmospheric, elevated or reduced pressure (for example from 0.5 to 5 bar). In general, the reactions are carried out at atmospheric pressure.

The compounds of the formula (I) according to the invention, in which Z represents a group of the formula

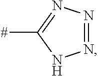

can be prepared by reacting compounds of the formula (VII-A), (VII-B), (VII-C) or (VII-D) in which Z¹ represents cyano in an inert solvent with an alkali metal azide in the presence of ammonium chloride or with trimethylsilyl azide, if appropriate in the presence of a catalyst.

Inert solvents for this reaction are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, or other solvents, such as dimethyl sulfoxide, dimethylformamide, N,N′-dimethylpropyleneurea (DMPU) or N-methylpyrrolidone (NMP). It is also possible to use mixtures of the solvents mentioned. Preference is given to using toluene.

A suitable azide reagent is in particular sodium azide in the presence of ammonium chloride or trimethylsilyl azide. The latter reaction can advantageously be carried out in the presence of a catalyst. Suitable for this purpose are in particular compounds such as di-n-butyltin oxide, trimethylaluminum or zinc bromide. Preference is given to using trimethylsilyl azide in combination with di-n-butyltin oxide.

The reaction is generally carried out in a temperature range of from +50° C. to +150° C., preferably at from +60° C. to +110° C. The reaction can be carried out at atmospheric, elevated or reduced pressure (for example from 0.5 to 5 bar). In general, the reaction is carried out at atmospheric pressure.

The compounds of the formula (I) according to the invention, in which Z represents a group of the formula

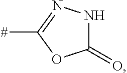

can be prepared by converting compounds of the formula (VII-A), (VII-B), (VII-C) or (VII-D) in which Z¹ represents methoxy- or ethoxycarbonyl initially in an inert solvent with hydrazine into compounds of the formula (VIII)

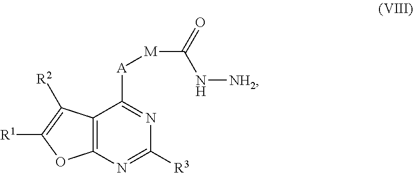

in which A, M, R¹, R² and R³ have the meanings given above,
and then reacting in an inert solvent with phosgene or a phosgene equivalent, such as, for example, N,N′-carbonyl diimidazole.

Suitable inert solvents for the first step of this reaction sequence are in particular alcohols, such as methanol, ethanol, n-propanol, isopropanol, n-butanol or tert-butanol, or ethers, such as diethyl ether, dioxane, tetrahydrofuran, glycol dimethyl ether or diethylene glycol dimethyl ether. It is also possible to use mixtures of these solvents. Preference is given to using a mixture of methanol and tetrahydrofuran. The second reaction step is preferably carried out in an ether, in particular in tetrahydrofuran. The reactions are generally carried out in a temperature range of from 0° C. to +70° C., under atmospheric pressure.

The compounds of the formula (I) according to the invention in which L¹ represents a group of the formula *-L^{1A}-V-L^{1B}-**, where L^{1A}, L^{1B} and V have the meanings given above, can alternatively also be prepared by converting compounds of the formula (IX)

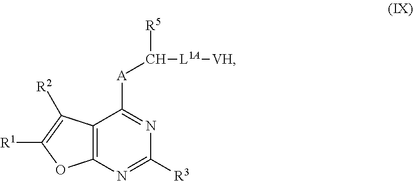

in which A, L^{1A}, V, R¹, R², R³ and R⁵ each have the meanings given above,
in the presence of a base, if appropriate in an inert solvent, with a compound of the formula (X)

X³-L^{1B}-Z¹ (X),

in which L^{1B} and Z¹ have the meanings given above
and
X³ represents a leaving group, such as, for example, halogen, mesylate or tosylate, or, in the case that L^{1B} represents —CH₂CH₂—, with a compound of the formula (XI)

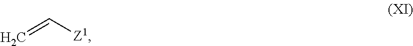

in which Z¹ has the meaning given above,
into compounds of the formula (VII-1)

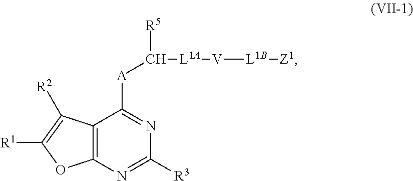

in which A, L^{1A}, L^{1B}, V, Z¹, R¹, R², R³ and R⁵ each have the meanings given above,
and then reacting these further, in a manner corresponding to the process described above.

For the process steps (IX)+(X) and (XI)→(VII-1), the reaction parameters described above for the reactions (II-A)+(III)→(IV-A) and (II-B)+(III)→(IV-B), such as solvents, bases and reaction temperatures, are used in an analogous manner.

The compounds of the formula (I) according to the invention in which L³ represents a group of the formula -W—CR⁹R¹⁰- or -W—CH₂—CR⁹R¹⁰-, where W, R⁹ and R¹⁰ have the meanings given above, can alternatively also be prepared by converting compounds of the formula (XII)

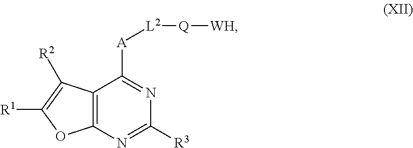

in which A, L², Q, W, R¹, R² and R³ each have the meanings given above,
in the presence of a base, if appropriate in an inert solvent, with a compound of the formula (XIII)

X³—(CH₂)ₘ—CR⁹R¹⁰—Z¹ (XIII),

in which R⁹, R¹⁰, X³ and Z¹ each have the meanings given above,
m represents the number 0 or 1,
or, in the case that L³ represents -W—CH₂CH₂-, with a compound of the formula (XI) into compounds of the formula (VII-2)

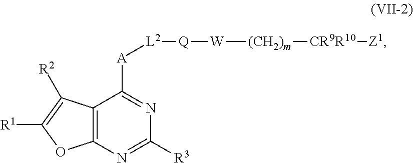

in which A, L², Q, W, Z¹, R¹, R², R³, R⁹, R¹⁰ and m each have the meanings given above,
and then reacting these further, in a manner corresponding to the process described above.

For the process steps (X)+(XIII) and (XI)→(VII-2), the reaction parameters described above for the reactions (II-A)+(III)→(IV-A) and (II-B)+(III)→(IV-B), such as solvents, bases and reaction temperatures, are used in an analogous manner.

Further compounds according to the invention can optionally also be prepared by conversions of functional groups of individual substituents, in particular those listed under R¹ and R², starting from the compounds of the formula (I) obtained by the above processes. These conversions are carried out by conventional methods known to the person skilled in the art and include, for example, reactions such as nucleophilic or electrophilic substitutions, oxidations, reductions, hydrogenations, transition metal-catalyzed coupling reactions, eliminations, alkylation, amination, esterifications, ester cleavage, etherification, ether cleavage, formation of carboxamides, and also the introduction and removal of temporary protective groups.

The compounds of the formulae (II-A), (II-B), (III), (VI-A), (VI-B), (VI-C), (VI-D) and (VI-E) are commercially available, known from the literature or can be prepared analogously to processes known from the literature (cf., for example, WO 03/018589; see also Reaction Schemes 1 and 2).

The preparation of the compounds according to the invention can be illustrated by the synthesis schemes below:

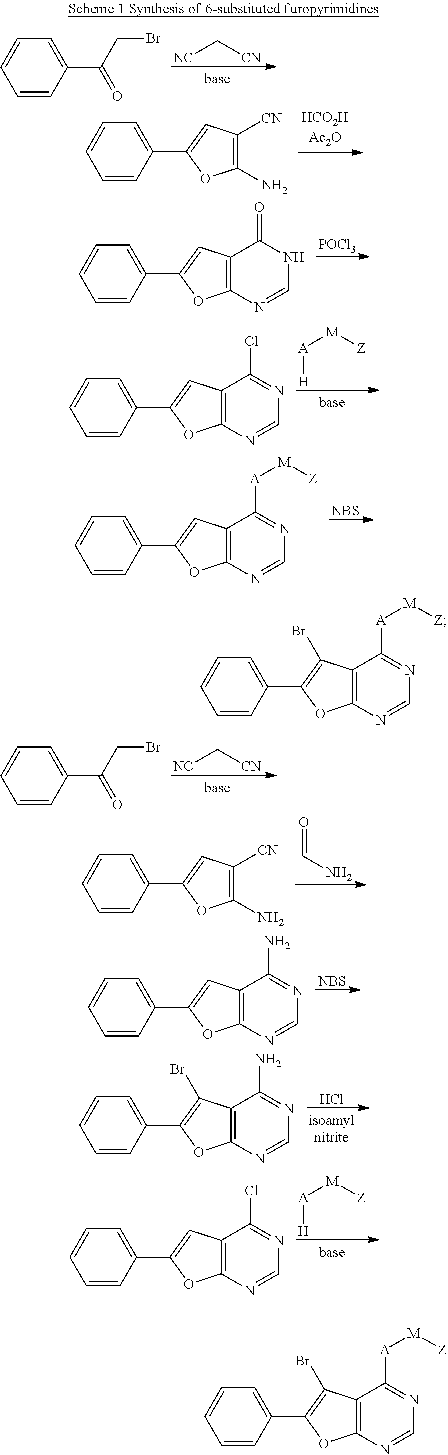

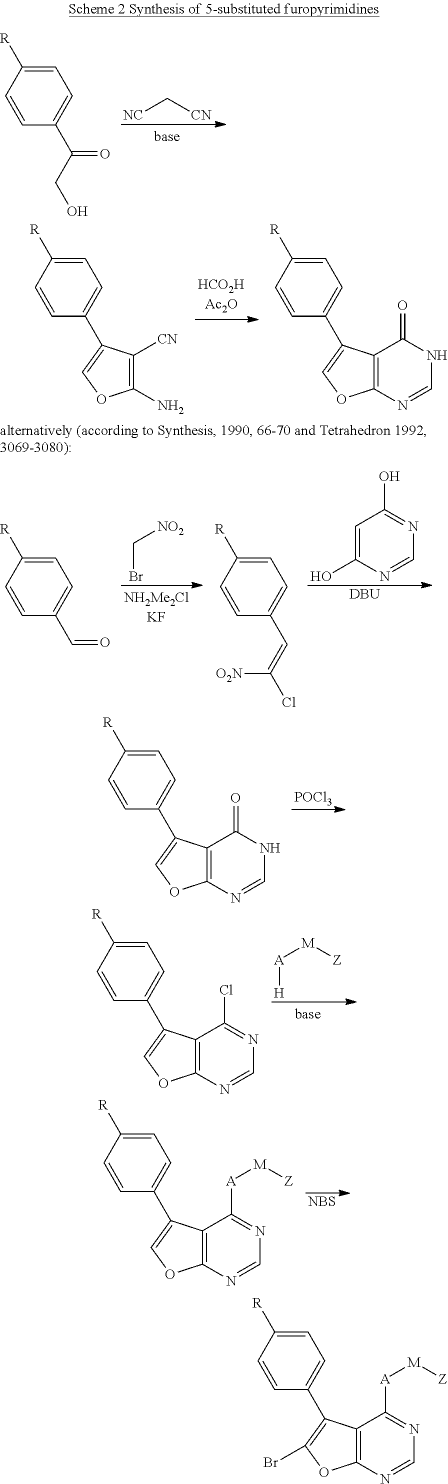

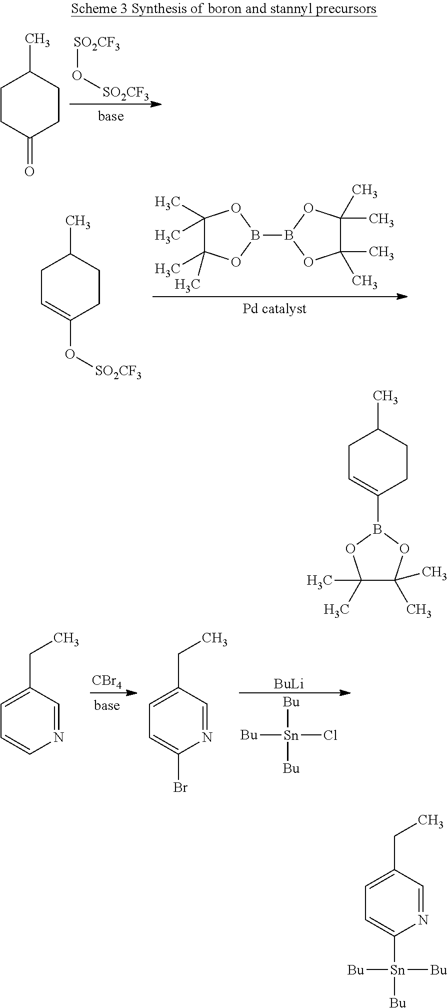

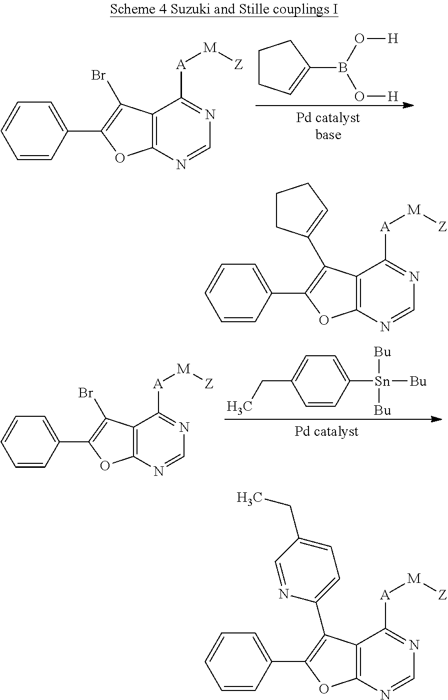

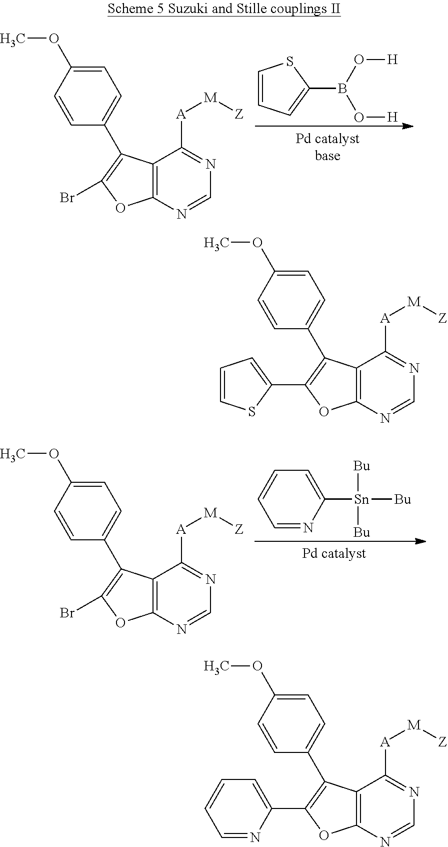

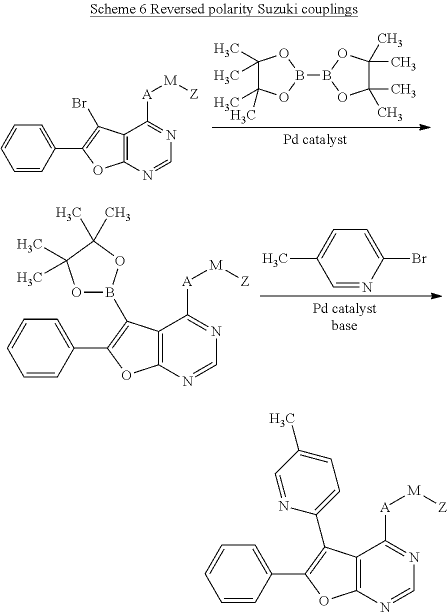

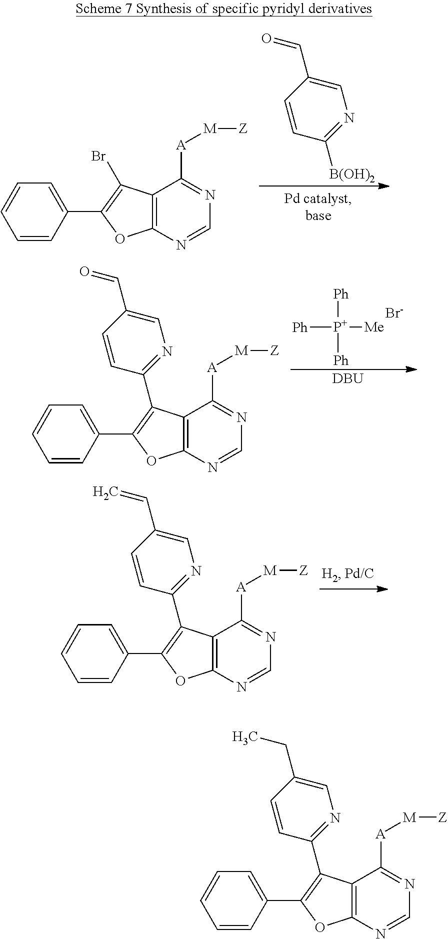

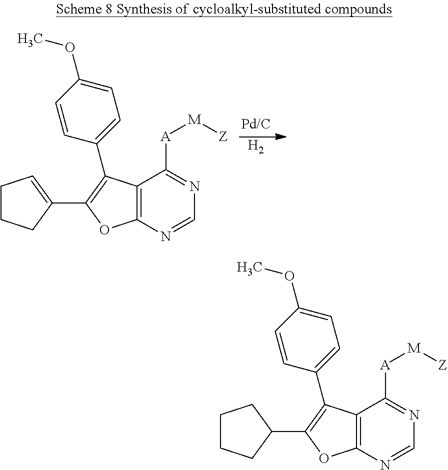

The compounds according to the invention possess valuable pharmacological properties and can be used for the prevention and treatment of diseases in humans and animals. The compounds according to the invention are chemically and metabolically stabile, non-prostanoid activators of the IP receptor.

They are thus suitable in particular for the prophylaxis and/or treatment of cardiovascular diseases such as stable and unstable angina pectoris, of hypertension and heart failure, pulmonary hypertension, for the prophylaxis and/or treatment of thromboembolic diseases and ischaemias such as myocardial infarction, stroke, transient and ischemic attacks and subarachnoid hemorrhage, and for the prevention of restenosis such as after thrombolytic treatments, percutaneous transluminal angioplasty (PTA), coronary angioplasty (PTCA) and bypass surgery.

The compounds according to the invention are particularly suitable for the treatment and/or prophylaxis of pulmonary hypertension (PH) including its various manifestations. The compounds of the invention are therefore particularly suitable for the treatment and/or prophylaxis of pulmonary arterial hypertension (PAH) and its subtypes such as idiopathic and familial pulmonary arterial hypertension, and the pulmonary arterial hypertension which is associated for example with portal hypertension, fibrotic disorders, HIV infection or inappropriate medications or toxins.

The compounds of the invention can also be used for the treatment and/or prophylaxis of other types of pulmonary hypertension. Thus, for example, they can be employed for the treatment and/or prophylaxis of pulmonary hypertension associated with left atrial or left ventricular disorders and with left heart valve disorders. In addition, the compounds of the invention are suitable for the treatment and/or prophylaxis of pulmonary hypertension associated with chronic obstructive pulmonary disease, interstitial pulmonary disease, pulmonary fibrosis, sleep apnea syndrome, disorders with alveolar hypoventilation, altitude sickness and pulmonary development impairments.

The compounds of the invention are furthermore suitable for the treatment and/or prophylaxis of pulmonary hypertension based on chronic thrombotic and/or embolic disorders such as, for example, thromboembolism of the proximal pulmonary arteries, obstruction of the distal pulmonary arteries and pulmonary embolism. The compounds of the invention can further be used for the treatment and/or prophylaxis of pulmonary hypertension connected with sarcoidosis, histiocytosis X or lymphangioleiomyomatosis, and where the pulmonary hypertension is caused by external compression of vessels (lymph nodes, tumor, fibrosing mediastinitis).

In addition, the compounds according to the invention can also be used for the treatment and/or prophylaxis of peripheral and cardial vascular diseases, peripheral occlusive diseases (PAOD, PVD) and disturbances of peripheral blood flow.

Furthermore, the compounds according to the invention can be used for the treatment of arteriosclerosis, hepatitis, asthmatic diseases, chronic obstructive pulmonary diseases (COPD), pulmonary edema, fibrosing lung diseases such as idiopathic pulmonary fibrosis (IPF) and ARDS, inflammatory vascular diseases such as scleroderma and lupus erythematosus, renal failure, arthritis and osteoporosis, and also for the prophylaxis and/or treatment of cancers, especially of metastasizing tumors.

Moreover, the compounds according to the invention can also be used as an addition to the preserving medium of an organ transplant, e.g. kidneys, lungs, heart or islet cells.

The present invention further relates to the use of the compounds according to the invention for the treatment and/or prophylaxis of diseases, and especially of the aforementioned diseases.

The present invention further relates to the use of the compounds according to the invention for the production of a medicinal product for the treatment and/or prophylaxis of diseases, and especially of the aforementioned diseases.

The present invention further relates to a method for the treatment and/or prophylaxis of diseases, especially of the aforementioned diseases, using an effective amount of at least one of the compounds according to the invention.

The present invention further relates to the compounds according to the invention for use in a method for the treatment and/or prophylaxis of angina pectoris, pulmonary hypertension, thromboembolic disorders and peripheral occlusive diseases.

The compounds of the invention can be employed alone or, if required, in combination with other active ingredients. The present invention further relates to medicaments comprising at least one of the compounds of the invention and one or more further active ingredients, especially for the treatment and/or prophylaxis of the aforementioned disorders. Suitable active ingredients for combinations are by way of example and preferably:
organic nitrates and NO donors such as, for example, sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil;
NO-independent but heme-dependent stimulators of guanylate cyclase such as in particular the compounds described in WO 00/06568, WO 00/06569, WO 02/42301 and WO 03/095451;
NO- and heme-independent activators of guanylate cyclase, such as in particular the compounds described in WO 01/19355, WO 01/19776, WO 01/19778, WO 01/19780, WO 02/070462 and WO 02/070510;
compounds which inhibit human neutrophile elastase (HNE), such as, for example, sivelestat, DX-890 (Reltran), elafin or in particular the compounds described in WO 03/053930, WO 2004/020410, WO 2004/020412, WO 2004/024700, WO 2004/024701, WO 2005/080372, WO 2005/082863 and WO 2005/082864;
compounds which inhibit the signal transduction cascade, for example and preferably from the group of kinase inhibitors, in particular from the group of tyrosine kinase and/or serine/threonine kinase inhibitors;
compounds which inhibit soluble epoxide hydrolase (sEH), such as, for example, N,N′-dicyclohexylurea, 12-(3-adamantan-1-yl-ureido)dodecanoic acid or 1-adamantan-1-yl-3-{5-[2-(2-ethoxyethoxy)ethoxy]pentyl}urea;
compounds which influence the energy metabolism of the heart, such as by way of example and preferably etomoxir, dichloroacetate, ranolazine or trimetazidine;
agonists of VPAC receptors, such as by way of example and preferably the vasoactive intestinal polypeptide (VIP);
agents having an antithrombotic effect, for example and preferably from the group of platelet aggregation inhibitors, of anticoagulants or of profibrinolytic substances;
active ingredients which lower blood pressure, for example and preferably from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors and diurectics; and/or
active ingredients which alter lipid metabolism, for example and preferably from the group of thyroid receptor agonists, cholesterol synthesis inhibitors such as by way of example and preferably HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile adsorbents, bile acid reabsorption inhibitors and lipoprotein(a) antagonists.

In a preferred embodiment of the invention, the compounds of the invention are administered in combination with a kinase inhibitor such as by way of example and preferably canertinib, imatinib, gefitinib, erlotinib, lapatinib, lestaurtinib, lonafarnib, pegaptinib, pelitinib, semaxanib, tandutinib, tipifarnib, vatalanib, sorafenib, sunitinib, bortezomib, lonidamine, leflunomide, fasudil, or Y-27632.

Agents having an antithrombotic effect preferably mean compounds from the group of platelet aggregation inhibitors, of anticoagulants or of profibrinolytic substances.

In a preferred embodiment of the invention, the compounds of the invention are administered in combination with a platelet aggregation inhibitor such as by way of example and preferably aspirin, clopidogrel, ticlopidine or dipyridamole.

In a preferred embodiment of the invention, the compounds of the invention are administered in combination with a thrombin inhibitor such as by way of example and preferably ximelagatran, melagatran, bivalirudin or clexane.

In a preferred embodiment of the invention, the compounds of the invention are administered in combination with a GPIIb/IIIa antagonist such as by way of example and preferably tirofiban or abciximab.

In a preferred embodiment of the invention, the compounds of the invention are administered in combination with a factor Xa inhibitor such as by way of example and preferably rivaroxaban, DU-176b, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or a low molecular weight (LMW) heparin derivative.

In a preferred embodiment of the invention, the compounds of the invention are administered in combination with a vitamin K antagonist such as by way of example and preferably coumarin.

Agents which lower blood pressure preferably mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors, and diuretics.

In a preferred embodiment of the invention, the compounds of the invention are administered in combination with a calcium antagonist such as by way of example and preferably nifedipine, amlodipine, verapamil or diltiazem.

In a preferred embodiment of the invention, the compounds of the invention are administered in combination with an alpha-1 receptor blocker such as by way of example and preferably prazosin.

In a preferred embodiment of the invention, the compounds of the invention are administered in combination with a beta-receptor blocker such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin All antagonist such as by way of example and preferably losartan, candesartan, valsartan, telmisartan or embusartan.

In a preferred embodiment of the invention, the compounds of the invention are administered in combination with an ACE inhibitor such as by way of example and preferably enalapril, captopril, lisinopril, ramipril, delapril, fosinopril, quinopril, perindopril or trandopril.

In a preferred embodiment of the invention, the compounds of the invention are administered in combination with an endothelin antagonist such as by way of example and preferably bosentan, darusentan, ambrisentan or sitaxsentan.

In a preferred embodiment of the invention, the compounds of the invention are administered in combination with a renin inhibitor such as by way of example and preferably aliskiren, SPP-600 or SPP-800.

In a preferred embodiment of the invention, the compounds of the invention are administered in combination with a mineralocorticoid receptor antagonist such as by way of example and preferably spironolactone or eplerenone.

In a preferred embodiment of the invention, the compounds of the invention are administered in combination with a Rho kinase inhibitor such as by way of example and preferably fasudil, Y-27632, SLx-2119, BF-66851, BF-66852, BF-66853, KI-23095, SB-772077, GSK-269962A or BA-1049.

In a preferred embodiment of the invention, the compounds of the invention are administered in combination with a diuretic such as by way of example and preferably furosemide.

Agents which alter lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the compounds of the invention are administered in combination with a CETP inhibitor such as by way of example and preferably torcetrapib (CP-529 414), JJT-705 or CETP vaccine (Avant).

In a preferred embodiment of the invention, the compounds of the invention are administered in combination with a thyroid receptor agonist such as by way of example and preferably D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins such as by way of example and preferably lovastatin, simvastatin, pravastatin, fluvastatin, atorvastatin, rosuvastatin, cerivastatin or pitavastatin.

In a preferred embodiment of the invention, the compounds of the invention are administered in combination with a squalene synthesis inhibitor such as by way of example and preferably BMS-188494 or TAK-475.

In a preferred embodiment of the invention, the compounds of the invention are administered in combination with an ACAT inhibitor such as by way of example and preferably avasimibe, melinamide, pactimibe, eflucimibe or SMP-797.

In a preferred embodiment of the invention, the compounds of the invention are administered in combination with an MTP inhibitor such as by way of example and preferably implitapide, BMS-201038, R-103757 or JTT-130.

In a preferred embodiment of the invention, the compounds of the invention are administered in combination with a PPAR-gamma agonist such as by way of example and preferably pioglitazone or rosiglitazone.

In a preferred embodiment of the invention, the compounds of the invention are administered in combination with a PPAR-delta agonist such as by way of example and preferably GW-501516 or BAY 68-5042.

In a preferred embodiment of the invention, the compounds of the invention are administered in combination with a cholesterol absorption inhibitor such as by way of example and preferably ezetimibe, tiqueside or pamaqueside.

In a preferred embodiment of the invention, the compounds of the invention are administered in combination with a lipase inhibitor such as by way of example and preferably orlistat.

In a preferred embodiment of the invention, the compounds of the invention are administered in combination with a polymeric bile acid adsorbent such as by way of example and preferably cholestyramine, colestipol, colesolvam, CholestaGel or colestimide.

In a preferred embodiment of the invention, the compounds of the invention are administered in combination with a bile acid reabsorption inhibitor such as by way of example and preferably ASBT(=IBAT) inhibitors such as, for example, AZD-7806, S-8921, AK-105, BARI-1741, SC-435 or SC-635.

In a preferred embodiment of the invention, the compounds of the invention are administered in combination with a lipoprotein(a) antagonist such as by way of example and preferably gemcabene calcium (CI-1027) or nicotinic acid.

The present invention further relates to medicaments comprising at least one of the compounds according to the invention, usually in combination with one or more inert, non-toxic, pharmaceutically suitable excipients, and their use for the purposes mentioned above.

The compounds of the invention may have systemic and/or local effects. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in a modified manner, and which contain the compounds of the invention in crystalline and/or amorphized and/or dissolved form, such as, for example, tablets (uncoated and coated tablets, for example having coatings which are resistant to gastric juice or are insoluble or dissolve with a delay and control the release of the compound of the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other routes of administration are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears and eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

Oral or parenteral administration are preferred, especially oral and intravenous administration.

The compounds of the invention can be converted into the stated administration forms. This can take place in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorings (e.g. inorganic pigments such as, for example, iron oxides) and masking flavors and/or odors.

It has generally proved to be advantageous on parenteral administration to administer amounts of about 0.001 to 1 mg/kg, preferably about 0.01 to 0.5 mg/kg of body weight to achieve effective results. On oral administration, the dosage is about 0.01 to 100 mg/kg, preferably about 0.01 to 20 mg/kg, and very particularly preferably 0.1 to 10 mg/kg of body weight.

It may nevertheless be necessary where appropriate to deviate from the stated amounts, in particular as a function of body weight, administration route, individual response to the active ingredient, type of preparation and time or interval over which administration takes place. Thus, in some cases it may be sufficient to make do with less than the aforementioned minimum amount, whereas in other cases the upper limit mentioned must be exceeded. Where relatively large amounts are administered, it may be advisable to distribute these in a plurality of single doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data of liquid/liquid solutions are based in each case on the volume.

### A. EXAMPLES

### Abbreviations

abs. absolute
Ac acetyl
Ac₂O acetic anhydride
Boc tert-butoxycarbonyl
Bu butyl
c concentration
conc. concentrated
DBU 1,8-diazabicyclo[5.4.0]undec-7-ene
DCI direct chemical ionization (in MS)
DIBAH diisobutylaluminum hydride
DIEA diisopropylethylamine (“Hünig base”)

### DMAP 4-N,N-dimethylaminopyridine

DME 1,2-dimethoxyethane

### DMF N,N-dimethylformamide

DMSO dimethyl sulfoxide
ee enantiomeric excess
EI electron impact ionization (in MS)
ESI electrospray ionization (in MS)
Et ethyl
GC gas chromatography
h hour(s)
HPLC high-performance liquid chromatography
LC-MS liquid chromatography-coupled mass spectrometry
m.p. melting point
Me methyl
min minute(s)
Ms methanesulfonyl(mesyl)
MS mass spectrometry

### NBS N-bromosuccinimide

NMR nuclear magnetic resonance spectrometry
Pd/C palladium on carbon
rac. racemic
RP reversed phase (in HPLC)
RT room temperature
Rₜ retention time (in HPLC)
sat. saturated
TFA trifluoroacetic acid
THF tetrahydrofuran
TLC thin-layer chromatography

### LC-MS, HPLC and GC Methods:

### Method 1 (HPLC):

Instrument: HP 1100 with DAD detection; column: Kromasil 100 RP-18, 60 mm×2.1 mm, 3.5 μm; mobile phase A: 5 ml of HClO₄ (70% strength)/liter of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

### Method 2 (LC-MS):

Instrument: Micromass Quattro LCZ with HPLC Agilent series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 3 (LC-MS):

MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A 2.5 min 30% A 3.0 min 5% A 4.5 min 5% A; flow rate: 0.0 min 1 ml/min, 2.5 min/3.0 min/4.5 min 2 ml/min; oven: 50° C.; UV detection: 210 nm.

### Method 4 (LC-MS):

Instrument: Micromass Platform LCZ with HPLC Agilent series 1100; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 5 (LC-MS):

MS instrument type: Waters ZQ; HPLC instrument type: Waters Alliance 2795; column: Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A 2 min 65% A 4.5 min 5% A 6 min 5% A; flow rate: 2 ml/min; oven: 40° C.; UV detection: 210 nm.

### Method: 6 (LC-MS):

MS instrument type: Micromass ZQ; HPLC instrument type: Waters Alliance 2795; column: Phenomenex Synergi 2.5 μMAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A 0.1 min 90% A 3.0 min 5% A 4.0 min 5% A 4.01 min 90% A; flow rate: 2 ml/min; oven: 50° C.; UV detection: 210 nm.

### Method 7 (LC-MS):

MS instrument type: Micromass ZQ; HPLC instrument type: HP 1100 series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 8 (LC-MS):

Instrument: Micromass Quattro LCZ with HPLC Agilent series 1100; column: Phenomenex Gemini 3μ, 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 9 (GC-MS):

Instrument: Micromass GCT, GC6890; column: Restek RTX-35, 15 m×200 μm×0.33 μm; constant helium flow rate: 0.88 ml/min; oven: 70° C.; inlet: 250° C.; gradient: 70° C., 30° C./min→310° C. (maintained for 3 min).

### Method 10 (LC-MS):

Instrument: Micromass Quattro LCZ with HPLC Agilent series 1100; column: Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A 2 min 65% A 4.5 min 5% A 6 min 5% A; flow rate: 2 ml/min; oven: 40° C.; UV detection: 208-400 nm.

### Method 11 (LC-MS):

Instrument: Micromass Quattro LCZ with HPLC Agilent series 1100; column: Phenomenex Synergi 2.5 μMAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A 0.1 min 90% A 3.0 min 5% A 4.0 min 5% A 4.1 min 90% A; flow rate: 2 ml/min; oven: 50° C.; UV detection: 208-400 nm.

### Method 12 (LC-MS):

Instrument: Micromass Quattro Micro MS with HPLC Agilent series 1100; column: Thermo Hypersil GOLD 3μ 20×4 mm; mobile phase A: 1 l of water+0.5 ml of 50% strength formic acid, mobile phase B: 1 l of acetonitrile+0.5 ml of 50% strength formic acid; gradient: 0.0 min 100% A 3.0 min 10% A 4.0 min 10% A 4.0 1 100% A (flow rate 2.5 ml) 5.00 100% A. oven: 50° C.; flow rate: 2 ml/min; UV detection: 210 nm.

### Method 13 (HPLC):

Instrument: HP 1100 with DAD detection; column: Kromasil 100 RP-18, 60 mm×2.1 mm, 3.5 μm; mobile phase A: 5 ml of HClO₄ (70% strength)/liter of water, mobile phase B: acetonitrile; gradient: 0 min 2% B→0.5 min 2% B→4.5 min 90% B→9.0 min 90% B→9.2 min 2% B→10 min 2% B; flow rate: 0.75 ml/min; column temperature: 30° C.; UV detection: 210 nm.

### Method 14 (LC-MS):

Instrument: Micromass Quattro Premier with Waters HPLC Acquity; column: Thermo Hypersil GOLD; 1.9μ50×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; oven: 50° C.; flow rate: 0.33 ml/min; UV detection: 210 nm.

### Example 1A

### 2-Amino-5-phenyl-3-furonitrile

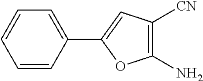

At RT, 68.6 ml (663 mmol) of diethylamine are added dropwise (cooling required to maintain the temperature) to a mixture of 60.0 g (301 mmol) of bromoacetophenone and 25.89 g (391.86 mmol) of malononitrile in 130 ml of DMF. Cooling is removed toward the end of the addition, and the mixture is stirred at RT for 1 h and then poured into 385 ml of water. The mixture is diluted with a further 125 ml of water and stirred at RT for 20 min. The precipitated solid is filtered off with suction, washed twice with in each case 125 ml of water, filtered off with suction to dryness and washed with petroleum ether. The residue is dried under high vacuum. This gives 33.3 g (50.1% of theory) of the target compound as crystals.

HPLC (method 1): Rₜ=4.27 min

MS (DCI): m/z=202 (M+NH₄)⁺, 185 (M+H)⁺

¹H-NMR (400 MHz, CDCl₃): δ=7.51-7.45 (m, 2H), 7.39-7.32 (m, 3H), 6.54 (s, 1H), 4.89 (br. s, 1H).

### Example 2A

### 6-Phenylfuro[2,3-d]pyrimidin-4(3H)-one

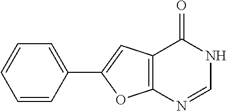

At 0° C., 424.5 ml (11.25 mol) of formic acid are added dropwise to 884.9 ml (9.378 mol) of acetic anhydride. The mixture is stirred at 0° C. for 30 min, and 69.1 g (0.375 mol) of 2-amino-5-phenyl-3-furonitrile are then added. Cooling is removed and the mixture is heated; evolution of gas commences at about 80° C. and ceases after about 3 h. The mixture is stirred under reflux for a total of 24 h (bath temperature about 130° C.). After cooling of the suspension to RT, 750 ml of diisopropyl ether are added, and the mixture is cooled to 0° C. and filtered off. The residue is washed with diisopropyl ether and dried under high vacuum. This gives 50.83 g (58.7% of theory) of the target compound as a solid.

HPLC (method 1): Rₜ=3.92 min

MS: m/z=213 (M+H)⁺

¹H-NMR (400 MHz, DMSO-d₆): δ=12.68 (br. s, 1H), 8.17 (s, 1H), 7.88 (d, 2H), 7.52-7.48 (m, 3H), 7.42-7.38 (m, 1H).

### Example 3A

### 4-Chloro-6-phenylfuro[2,3-d]pyrimidine

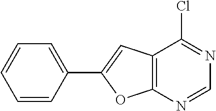

At RT, 50 g (235.6 mmol) of 6-phenylfuro[2,3-d]pyrimidin-4(3H)-one are suspended in 375 ml (4023 mmol) of phosphorus oxychloride, and the mixture is heated to the boil (evolution of HCl). After 1 h, the dark solution is cooled to RT and added dropwise to a vigorously stirred mixture of 1.25 liters of water and 2.25 liters of conc. ammonia solution (25% by weight) (temperature increase to 55-75° C., pH>9). After the end of the addition, the mixture is cooled to RT and extracted three times with in each case 1.6 liters of dichloromethane. The combined organic phases are dried and concentrated under reduced pressure. The residue is triturated with diethyl ether, filtered off with suction and dried under high vacuum. This gives 47.3 g (87% of theory) of the target compound.

HPLC (method 1): Rₜ=4.67 min

MS: m/z=231 (M+H)⁺

¹H-NMR (300 MHz, DMSO-d₆): δ=8.84 (s, 1H), 8.05 (m, 2H), 7.77 (s, 1H), 7.61-7.50 (m, 3H).

### Example 4A

### Methyl 6-[(6-phenylfuro[2,3-d]pyrimidin-4-yl)amino]hexanoate

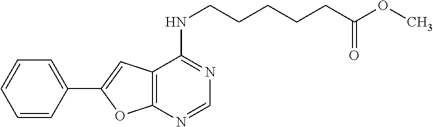

2.0 g (8.67 mmol) of 4-chloro-6-phenylfuro[2,3-d]pyrimidine and 6.04 ml (34.7 mmol) of DIEA in 5 ml of DMF are heated to 160° C. 3.15 g (17.34 mmol) of methyl 6-aminohexanoate hydrochloride are added, and the mixture is stirred at 160° C. for 4 h. After cooling, the mixture is added to ice-water and extracted three times with ethyl acetate. The combined organic phases are washed with saturated ammonium chloride solution, dried over magnesium sulfate and concentrated under reduced pressure. Methanol is added to the oil that remained. The precipitated solid is filtered off with suction, the filter cake is washed with methanol and the solid is dried under high vacuum. This gives 1.85 g (57.2% of theory) of the target compound.

LC-MS (method 2): Rₜ=2.38 min.; m/z=340 (M+H)⁺

¹H-NMR (400 MHz, DMSO-d₆): δ=8.24 (s, 1H), 7.98 (br. s, 1H), 7.79 (d, 2H), 7.51 (t, 2H), 7.43-7.37 (m, 2H), 3.59 (s, 3H), 3.49 (q, 2H), 2.32 (t, 2H), 1.65-1.56 (m, 4H), 1.41-1.35 (m, 2H).

### Example 5A

### Methyl 6-[(5-bromo-6-phenylfuro[2,3-d]pyrimidin-4-yl)amino]hexanoate

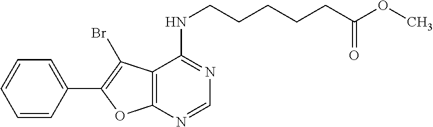

1.75 g (5.15 mmol) of methyl 6-[(6-phenylfuro[2,3-d]pyrimidin-4-yl)amino]hexanoate are initially charged in 5.2 ml of carbon tetrachloride. 1.054 g (5.92 mmol) of N-bromosuccinimide are added at RT, and the mixture is then heated under reflux for about 1 h. After cooling, the mixture is concentrated under reduced pressure and the residue is chromatographed on silica gel (mobile phase: cyclohexane/ethyl acetate 4:1). This gives 0.89 g (41.2% of theory) of the target compound.

LC-MS (method 3): Rₜ=2.64 min.; m/z=420 (M+H)⁺

¹H-NMR (400 MHz, DMSO-d₆): δ=8.33 (s, 1H), 8.02 (d, 2H), 7.61-7.49 (m, 3H), 7.04 (t, 1H), 3.59 (s, 3H), 3.59-3.52 (m, 2H), 2.31 (t, 2H), 1.68-1.54 (m, 4H), 1.40-1.31 (m, 2H).

### Example 6A

### 6-Phenylfuro[2,3-d]pyrimidine-4-amine

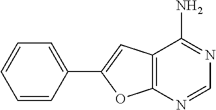

110 g (597 mmol) of 2-amino-5-phenyl-3-furonitrile are suspended in 355 ml (9 mol) of formamide and heated for 1.5 h (bath temperature about 210° C.). The mixture is then cooled to RT and stirred into water. The precipitated solid is filtered off with suction and washed with water. The moist product is triturated with dichloromethane, again filtered off with suction and dried under reduced pressure. This gives 106 g (80% of theory) of the target compound.

LC-MS (method 4): Rₜ=3.1 min.; m/z=212 (M+H)⁺

HPLC (method 1): Rₜ=3.63 min.

¹H-NMR (400 MHz, DMSO-d₆): δ=8.20 (s, 1H), 7.8 (d, 2H), 7.55-7.32 (m, 6H).

### Example 7A

### 5-Bromo-6-phenylfuro[2,3-d]pyrimidine-4-amine

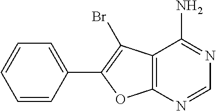

80 g (378.7 mmol) of 6-phenylfuro[2,3-d]pyrimidine-4-amine in 770 ml of carbon tetrachloride are heated to 60° C. 84.3 g (473.4 mmol) of N-bromosuccinimide are added, and the mixture is stirred under reflux overnight. After cooling, the mixture is filtered off, and the filter cake is triturated successively with dichloromethane and acetonitrile and again filtered off. The filter cake is then dried under reduced pressure. This gives 86 g of the target product (78.2% of theory).

MS (DCI): m/z=290/292 (M+H)⁺

¹H-NMR (400 MHz, DMSO-d₆): δ=8.28 (s, 1H), 8.03 (d, 2H), 7.60-7.50 (m, 5H).

### Example 8A

### 5-Bromo-4-chloro-6-phenylfuro[2,3-d]pyrimidine

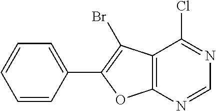

54 g (186 mmol) of 5-bromo-6-phenylfuro[2,3-d]pyrimidine-4-amine are initially charged in 135 ml of chloroform, 70 ml of 4 N hydrogen chloride in dioxane (280 mmol) are added and the mixture is heated to reflux. 50 ml (372 mmol) of isoamyl nitrite are added dropwise with evolution of gas. After the addition has ended, the mixture is stirred at reflux for 3 h, and the cooled reaction mixture is then added to water and extracted with dichloromethane. The organic phase is washed with saturated sodium bicarbonate solution, dried over sodium sulfate and concentrated under reduced pressure. The crude product is purified by chromatography on silica gel (mobile phase: dichloromethane). For further purification, the product is triturated with methanol, filtered off with suction and dried under high vacuum. This gives 32 g of the target product (55.5% of theory). LC-MS (method 3): Rₜ=2.54 min.; m/z=309/310 (M+H)⁺

HPLC (method 1): Rₜ=5.08 min.

¹H-NMR (400 MHz, CDCl₃): δ=8.79 (s, 1H), 8.23-8.20 (m, 2H), 7.58-7.51 (m, 3H).

### Example 9A

### Methyl 6-{[6-phenyl-5-(4,4,5,5-tetramethyl-1,3,2-dioxaborolan-2-yl)furo[2,3-d]pyrimidin-4-yl]amino}hexanoate

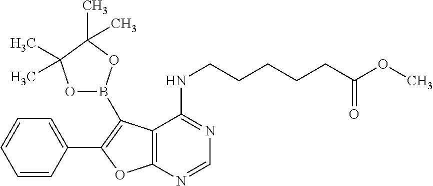

Under an atmosphere of argon, 455 mg (1.79 mmol) of 4,4,4′,4′,5,5,5′,5′-octamethyl-2,2′-bi-1,3,2-dioxaborolane are initially charged, and 600 mg (1.43 mmol) of methyl 6-[(5-bromo-6-phenylfuro[2,3-d]pyrimidin-4-yl)amino]hexanoate, 422 mg (4.30 mmol) of potassium acetate, 5 ml of DMSO and 70 mg (0.09 mmol) of 1,1-bis(diphenylphosphino)ferrocene]palladium(II) chloride are added in succession. The reaction is stirred at 90° C. for 7 h. For work-up, the reaction mixture is diluted with DMSO and the product is isolated by preparative RP-HPLC (gradient of water and acetonitrile). This gives 515 mg (29% of theory) of the target compound.

LC-MS (method 3): Rₜ=2.85 min; m/z=466 (M+H)⁺.

¹H-NMR (400 MHz, DMSO-d₆): δ=8.29 (s, 1H), 7.89 (dd, 2H), 7.34 (t, 1H), 7.55-7.45 (m, 3H), 3.61-3.50 (m, 5H: including 3.58 (s, 3H)), 2.33 (t, 2H), 1.71-1.56 (m, 4H), 1.48-1.28 (m, 14H: including 1.35 (s, 12H)).

### Example 10A

### tert-butyl (2E,6R)-6-hydroxyhept-2-enoate

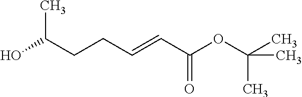

Solution A: 10.71 g (267.7 mmol) of 60% sodium hydride are suspended in 150 ml of abs. THF, and 43.3 ml (276.7 mmol) of tert-butyl P,P-dimethylphosphonoacetate are added dropwise with cooling. The mixture is stirred at RT, and after about 30 min a solution is formed.

187.4 ml (187.4 mmol) of a 1 M solution of DIBAH in THF are added dropwise to a solution, cooled to −78° C., of 17.87 g (178.5 mmol) of (R)-γ-valerolactone [(5R)-5-methyl-dihydrofuran-2(3H)-one] in 200 ml of abs. THF. The solution is stirred at −78° C. for 1 h, and solution A prepared above is then added. After the addition, the mixture is slowly warmed to RT and stirred at RT overnight. The reaction mixture is added to 300 ml of ethyl acetate and extracted with 50 ml of concentrated potassium sodium tartrate solution. After phase separation, the aqueous phase is reextracted with ethyl acetate. The organic phases are combined, washed with saturated sodium chloride solution, dried over magnesium sulfate and concentrated under reduced pressure. The residue is purified by chromatography on silica gel (mobile phase: cyclohexane/ethyl acetate 5:1). This gives 32.2 g (90.1% of theory) of the target product which contains small amounts of the cis isomer.

MS (DCI): m/z=218 (M+NH₄)⁺

¹H-NMR (400 MHz, DMSO-d₆): δ=6.70 (dt, 1H), 5.73 (d, 1H), 4.44 (d, 1H), 3.58 (m, 1H), 2.28-2.13 (m, 2H), 1.47-1.40 (m, 2H), 1.45 (s, 9H), 1.04 (d, 3H).

### Example 11A

### tert-Butyl(−)-6-hydroxyheptanoate

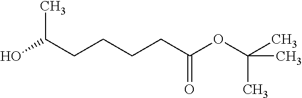

32.2 g (160.8 mmol) of tert-butyl (2E,6R)-6-hydroxyhept-2-enoate are dissolved in 200 ml of ethanol, and 1.7 g of 10% palladium on carbon are added. The mixture is stirred at RT under an atmosphere of hydrogen (atmospheric pressure) for 2 h and then filtered through Celite. The filtrate is concentrated under reduced pressure. The residue gives, after chromatography on silica gel (mobile phase: cyclohexane/ethyl acetate 10:1→6:1), 15.66 g of the target product (48.1% of theory).

MS (DCI): m/z=220 (M+NH₄)⁺

¹H-NMR (400 MHz, CDCl₃): δ=3.85-3.75 (m, 1H), 2.22 (t, 2H), 1.68-1.54 (m, 2H), 1.53-1.30 (m, 4H), 1.45 (s, 9H), 1.18 (d, 3H).

[α]_{D}²⁰=−21°, c=0.118, chloroform.

### Example 12A

### tert-Butyl (6R)-6-[(5-bromo-6-phenylfuro[2,3-d]pyrimidin-4-yl)oxy]heptanoate

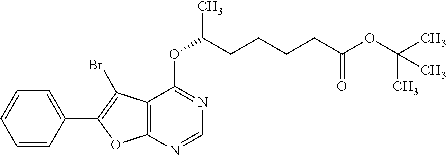

10.0 g (32.30 mmol) of 5-bromo-4-chloro-6-phenylfuro[2,3-d]pyrimidine and 10.8 g (53.30 mmol) of tert-butyl(−)-6-hydroxyheptanoate are initially charged in 20 ml of DMF, 2.1 g (53.30 mmol) of 60% sodium hydride are added at 0° C. and the mixture is warmed to RT and stirred at this temperature for 45 min. Water is added, and the reaction mixture is extracted with dichloromethane. The organic phase is washed with saturated sodium chloride solution, dried over sodium sulfate and concentrated under reduced pressure. The residue is chromatographed on silica gel using a gradient of cyclohexane and ethyl acetate (20/1→10/1). This gives 6.8 g of the target product (44% of theory) LC-MS (method 5): Rₜ=4.87 min; m/z=475 (M+H)⁺.

¹H-NMR (400 MHz, DMSO-d₆): δ=8.60 (s, 1H), 8.06 (d, 2H), 7.64-7.50 (m, 3H), 5.48 (m, 1H), 2.18 (t, 2H), 1.76 (m, 2H), 1.61-1.28 (m, 16H: including 1.33 (s, 9H)).

[α]_{D}²⁰=−56°, c=0.450, chloroform.

### Example 13A

### 5-Ethyl-2-(tributylstannyl)pyridine

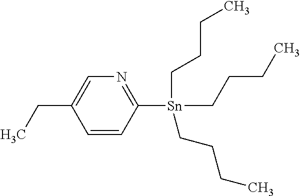

2250 mg (12 mmol) of 2-bromo-5-ethylpyridine [prepared analogously to J. Org. Chem., 2003, 2028 and Chem. Commun., 2000, 951] and 4330 mg (13.3 mmol) of tributyltin chloride are dissolved in 20 ml of THF, and 8.3 ml (13.3 mmol) of 1.6 N n-butyllithium in hexane are added dropwise at 0° C. The mixture is stirred at 0° C. for 2.5 h and at RT for 12 h. The reaction mixture is diluted with dichloromethane and washed with ammonium chloride solution and saturated sodium chloride solution, and the organic phase is dried over sodium sulfate and concentrated on a rotary evaporator. The crude product is chromatographed on silica gel (dichloromethane, then ethyl acetate). This gives 155 mg (25% of theory) of the title compound.

LC-MS (method 6): Rₜ=1.81 min; m/z=397 (M+H)⁺.

¹H-NMR (400 MHz, DMSO-d6): δ=8.55 (d, 1H), 7.46 (dd, 1H), 7.35 (d, 1H), 2.56 (q, 2H), 1.52 (t, 6H), 1.29 (m, 6H), 1.21-1.01 (m 6H), 0.87 (t, 9H), 0.83 (t, 3H).

### Example 14A

### 1-[(Z)-2-Chloro-2-nitrovinyl]-4-methoxybenzene

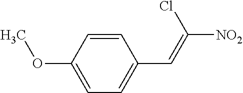

Analogously to a literature procedure [D. Dauzonne, Synthesis, 1990, 66-70], a mixture of 10.0 g (73.5 mmol) of 4-methoxybenzaldehyde, 9.0 ml (13.5 g, 96.2 mmol) of bromonitromethane, 53.9 g (661.0 mmol) of dimethylammonium chloride and 0.6 g (11.0 mmol) of potassium fluoride in 150 ml of xylene is stirred on a water separator at 160° C. for 15 hours. After addition of 25 ml of water and 100 ml of dichloromethane, the organic phase is separated off and the aqueous phase is extracted three times with in each case 100 ml of dichloromethane. The combined organic extracts are dried over sodium sulfate, filtered and concentrated under reduced pressure. The residue is chromatographed on silica gel (mobile phase: cyclohexane/dichloromethane 1:1). This gives 9.6 g (59% of theory) of the target compound.

LC-MS (method 7): Rₜ=2.52 min.

¹H-NMR (400 MHz, CDCl₃): δ=8.60 (s, 1H), 8.03 (d, 2H), 7.15 (d, 2H), 3.86 (s, 3H).

### Example 15A

### 5-(4-Methoxyphenyl)furo[2,3-d]pyrimidin-4(3H)-one

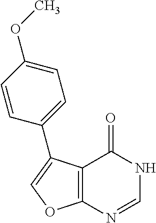

Analogously to a literature procedure [D. Dauzonne, Tetrahedron, 1992, 3069-3080], a suspension of 10.1 g (47.4 mmol) of 1-[(Z)-2-chloro-2-nitrovinyl]-4-methoxybenzene and 5.8 g (52.2 mmol) of 4,6-dihydroxypyrimidine in 200 ml of ethanol is stirred at 85° C. for ten minutes. 15.6 ml (15.9 g, 104.3 mmol) of 1,8-diazabicyclo[5.4.0]undec-7-ene are then added slowly. The mixture is stirred at this temperature for 15 h and then concentrated under reduced pressure. The residue is taken up in dichloromethane and chromatographed on silica gel (mobile phase: dichloromethane/methanol 95:5). The solid obtained is triturated with acetonitrile and filtered. This gives 2.3 g (20% of theory) of the target product.

LC-MS (method 3): Rₜ=1.57 min.; m/z=290 (M+H)⁺

¹H-NMR (400 MHz, CDCl₃): δ=12.66 (s, NH), 8.15 (s, 1H), 8.14 (s, 1H), 7.92 (d, 2H), 6.98 (d, 2H), 3.79 (s, 3H).

### Example 16A

### 4-Chloro-5-(4-methoxyphenyl)furo[2,3-d]pyrimidine

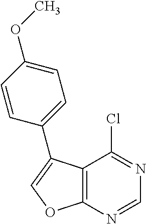

14.5 ml (13.6 g, 90.8 mmol) of N,N-diethylaniline are added to a suspension von 10.0 g (41.3 mmol) of 5-(4-methoxyphenyl)furo[2,3-d]pyrimidin-4(3H)-one in 250 ml of toluene, and the mixture is heated to 100° C. At this temperature, 4.2 ml (7.0 g, 45.4 mmol) of phosphoryl chloride are added dropwise, and the reaction mixture is stirred at 100° C. for 15 h. A further 1.2 ml (2.0 g, 13 mmol) of phosphoryl chloride are then added, and the reaction mixture is again stirred at 100° C. for 22 h. After cooling to room temperature, the reaction solution is washed successively quickly with 250 ml of ice-water, twice with in each case 250 ml of cold 20% strength sodium hydroxide solution, once more with 250 ml of ice-water, 250 ml of saturated sodium chloride solution, 1 N hydrochloric acid and 250 ml of ice-water. The organic phase is dried over sodium sulfate, filtered and concentrated under reduced pressure. This gives 6.3 g (59% of theory) of the title compound.

LC-MS (method 8): Rₜ=2.28 min.; m/z=261 (M+H)⁺

¹H-NMR (400 MHz, CDCl₃): δ=8.86 (s, 1H), 8.40 (s, 1H), 7.52 (d, 2H), 7.08 (d, 2H), 3.82 (s, 3H).

### Example 17A

### Methyl 6-{[5-(4-methoxyphenyl)furo[2,3-d]pyrimidin-4-yl]amino}hexanoate

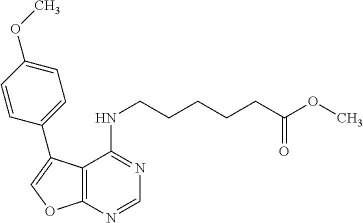

7.1 g (27.2 mol) of 4-chloro-5-(4-methoxyphenyl)furo[2,3-d]pyrimidine are dissolved in 250 ml of acetonitrile, and 5.9 g (32.7 mmol) of methyl 6-aminohexanoate hydrochloride and 9.4 g (68.1 mmol) of potassium carbonate are added. The mixture is heated under reflux for 18 hours and then, after cooling to room temperature, filtered. The residue is triturated with in each case 50 ml of water three times, filtered and dried under reduced pressure. This gives 4.1 g (41% of theory) of the title compound.

LC-MS (method 7): Rₜ=2.47 min.; m/z=370 (M+H)⁺

¹H-NMR (400 MHz, CDCl₃): δ=8.31 (s, 1H), 7.88 (s, 1H), 7.42 (d, 2H), 7.10 (d, 2H), 5.79 (t, 1H), 3.82 (s, 3H), 3.57 (s, 3H), 3.43 (q, 2H), 2.30 (t, 2H), 1.57-1.48 (m, 4H), 1.31-1.24 (m, 2H).

### Example 18A

### Methyl 6-{[6-bromo-5-(4-methoxyphenyl)furo[2,3-d]pyrimidin-4-yl]amino}hexanoate

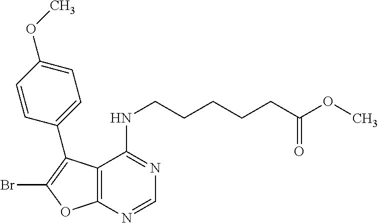

At room temperature, 4.1 g (11.1 mmol) of methyl 6-{[5-(4-methoxyphenyl)furo[2,3-d]pyrimidin-4-yl]amino}hexanoate are dissolved in 150 ml of carbon tetrachloride, and 2.2 g (12.2 mmol) of N-bromosuccinimide are added. The mixture is stirred under reflux for three hours and then, after cooling to room temperature, filtered, and the filtrate is concentrated under reduced pressure. This gives 4.8 g (96% of theory) of the title compound.

LC-MS (method 8): Rₜ=2.65 min.; m/z=448 (M+H)⁺

¹H-NMR (400 MHz, CDCl₃): δ=8.29 (s, 1H), 7.41 (d, 2H), 7.12 (d, 2H), 5.61 (t, 1H), 3.82 (s, 3H), 3.57 (s, 3H), 3.38 (q, 2H), 2.28 (t, 2H), 1.54-1.42 (m, 4H), 1.26-1.18 (m, 2H).

### Example 19A

### tert-Butyl (6R)-6-{[5-(4-methoxyphenyl)furo[2,3-d]pyrimidin-4-yl]oxy}heptanoate

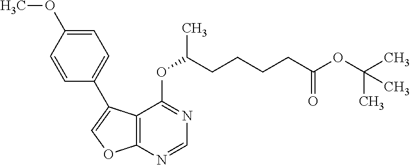

Under an atmosphere of argon, 5.0 g (18.90 mmol) of 4-chloro-5-(4-methoxyphenyl)furo[2,3-d]pyrimidine and 5.4 g (26.58 mmol) of tert-butyl(−)-6-hydroxyheptanoate are dissolved in 20 ml of THF, and 23.7 ml (23.73 mmol) of 1 M N′″-tert-butyl-N,N′,N″-tris[tris(dimethylamino)phosphoranylidene]phosphorimidic triamide solution in hexane are then added dropwise at −10° C. The mixture is stirred at −10° C. for 10 min, and the reaction mixture is slowly warmed to RT and stirred at this temperature for 2.5 h. Water is added, and the reaction mixture is neutralized with 1 M hydrochloric acid and extracted with dichloromethane. The organic phase is dried over sodium sulfate and concentrated under reduced pressure. The residue is chromatographed on silica gel using a gradient of cyclohexane and ethyl acetate 7/1→5/1. This gives 4.3 g (53% of theory) of the target compound.

LC-MS (method 5): Rₜ=4.38 min; m/z=427 (M+H)⁺.

1H-NMR (400 MHz, DMSO-d6): δ=8.57 (s, 1H), 8.25 (s, 1H), 7.68 (d, 2H), 7.01 (d, 2H), 5.45 (m, 1H), 3.81 (s, 3H), 2.13 (t, 2H), 1.67 (m, 2H), 1.49 (m, 2H), 1.32 (m, 14H).

[α]_{D}²⁰=−41°, c=0.575, chloroform.

### Example 20A

### tert-Butyl (6R)-6-{[6-bromo-5-(4-methoxyphenyl)furo[2,3-d]pyrimidin-4-yl]oxy}heptanoate

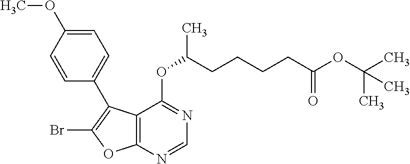

Under an atmosphere of argon, 2.1 g (4.92 mmol) of tert-butyl (6R)-6-{[5-(4-methoxyphenyl)furo[2,3-d]pyrimidin-4-yl]oxy}heptanoate are dissolved in 40 ml of acetonitrile, 1.0 g (5.42 mmol) of N-bromosucchinimide are added and the mixture is stirred at RT for 2 h. The reaction solution is concentrated under reduced pressure and the residue is pre-purified on silica gel using a mobile phase of cyclohexane and ethyl acetate 10/1. The concentrated product fractions are once more chromatographed on silica gel (Biotage® cartridge) (gradient cyclohexane and ethyl acetate 20/1→15/1→10/1). This gives 1.0 g (43% of theory) of the target compound.

LC-MS (method 5): Rₜ=4.66 min; m/z=505 (M+H)⁺.

¹H-NMR (400 MHz, DMSO-d₆): δ=8.57 (s, 1H), 7.52 (d, 2H), 7.05 (d, 2H), 5.35 (m, 1H), 3.82 (s, 3H), 2.11 (t, 2H), 1.57 (m, 2H), 1.47 (m, 2H), 1.34 (s, 9H), 1.31-1.12 (m, 5H: including 1.25 (d, 3H)).

[α]_{D}²⁰=−45°, c=0.515, chloroform.

### Example 21A

### (4-Ethylcyclohex-1-en-1-yl)-trifluoromethanesulfonate

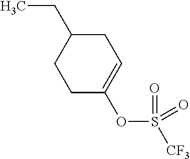

The compound is prepared according to the literature procedure Synthesis, 1980, 283-284.

GC-MS (method 9): Rₜ=3.17 min; m/z=258 (M)⁺.

¹H-NMR (400 MHz, DMSO-d₆): δ=5.85 (m, 1H), 2.44-2.21 (m, 3H), 1.90-1.75 (m, 2H), 1.50-1.23 (m, 4H), 0.88 (t, 3H).

The following trifluoromethanesulfonates are prepared in an analogous manner:

| Ex- | | |
|---|---|---|
| ample | Structure | Analytical data |
| 22A | 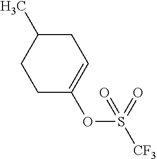 | GC-MS (method 9): Rₜ = 2.67; m/z = 244 (M)⁺. 1H-NMR (400 MHz, DMSO-d₆): δ = 5.83 (m, 1H), 2.44-2.18 (m, 3H), 1.85-1.73 (m, 2H), 1.65 (m, 1H), 1.39 (m, 1H), 0.94 (d, 3H). |
| 23A | 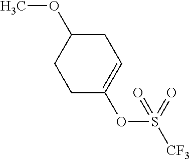 | GC-MS (method 9): Rₜ = 3.35 min; m/z = 260 (M)⁺. ¹H-NMR (400 MHz, DMSO-d₆): δ = 5.86 (m, 1H), 3.48 (m, 1H), 3.25 (s, 3H), 2.48-2.23 (m, 3H), 2.22-2.11 (m, 1H), 1.92-1.81 (m, 2H). |

### Example 24A

### 2-(4-Ethylcyclohex-1-en-1-yl)-4,4,5,5-tetramethyl-1,3,2-dioxaborolane

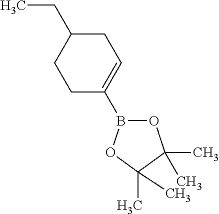

Under an atmosphere of argon, 1180 mg (4.57 mmol) of 4-ethylcyclohex-1-en-1-yl trifluoromethanesulfonate, 1276 g (5.03 mmol) of 4,4,4′,4′,5,5,5′,5′-octamethyl-2,2′-bi-1,3,2-dioxaborolane, 947 mg (6.85 mmol) of potassium carbonate, 72 mg (0.27 mmol) of triphenylphosphine and 96 mg (0.14 mmol) of bis(triphenylphosphine)palladium(II) chloride are initially charged in 3 ml of dioxane. The reaction mixture is stirred at 80° C. overnight. The reaction mixture is diluted with dichloromethane and washed with water and saturated sodium chloride solution, and the aqueous phase is extracted with dichloromethane. The combined organic phases are dried over sodium sulfate and concentrated on a rotary evaporator. The residue is dried under reduced pressure and chromatographed on silica gel using a mobile phase of cyclohexane and ethyl acetate 10/1. This gives 691 mg (60% of theory) of the target compound.

GC-MS (method 9): Rₜ=4.78 min; m/z=236 (M)⁺.

¹H-NMR (400 MHz, DMSO-d₆): δ=6.42 (m, 1H), 2.21-2.05 (m, 2H), 2.01-1.89 (m, 1H), 1.72-1.58 (m, 2H), 1.42-1.12 (m, 16H: including 1.25 (t, 2H) and 1.18 (s, 12H)).

The following boronic acid esters are prepared in an analogous manner:

| Example | Structure | Analytical data |
|---|---|---|
| 25A | 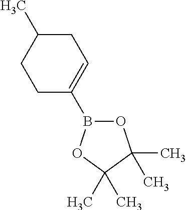 | GC-MS (method 9): Rₜ = 4.30 min.; m/z = 222 (M)⁺. ¹H-NMR (400 MHz, DMSO-d₆): δ = 6.42 (m, 1H), 2.18-1.90 (m, 3H), 1.71-1.45 (m, 4H), 1.18 (s, 12H), 0.91 (d, 3H). |
| 26A | 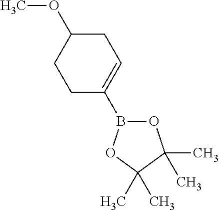 | MS (DCl, NH₃): m/z = 256 (M + NH₄)⁺. ¹H-NMR (400 MHz, DMSO-d₆): δ = 6.42 (m, 1H), 3.38 (m, 1H), 3.23 (s, 3H), 2.44-2.33 (m, 1H), 2.18-2.07 (m, 1H), 2.04-1.90 (m, 2H), 1.85-1.75 (m, 1H), 1.43 (m, 1H), 1.17 (s, 12H). |

### Example 27A

### Methyl 3-nitrophenoxyacetate

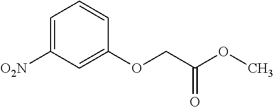

50 g (359.4 mmol) of 3-nitrophenol and 175.67 g (539 mmol) of cesium carbonate are initially charged in 1.0 liter of acetone, and 71.5 g (467.3 mmol) of methyl bromoacetate are added. The mixture is stirred at 50° C. for 1 h and, after cooling, poured into 7.5 liter of water. The suspension is stirred for 30 min and then filtered off with suction, and the filter residue is washed with water. The solid is dried in a drying cabinet at 50° C. and 100 mbar. This gives 64.3 g (84.7% of theory) of the target compound.

MS (DCI): m/z=229 (M+NH₄)⁺

¹H-NMR (300 MHz, CDCl₃): δ=7.90 (dd, 1H), 7.43 (t, 1H), 7.48 (t, 1H), 7.28 (dd, 1H), 4.75 (s, 2H), 3.86 (s, 3H).

### Example 28A

### Methyl 3-aminophenoxyacetate

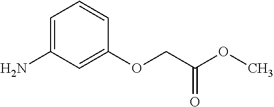

Under argon, 1.3 g of palladium on carbon (10%) are added to 13 g (61.6 mmol) of methyl 3-nitrophenoxyacetate in 150 ml of methanol. The mixture is stirred under an atmosphere of hydrogen (atmospheric pressure) at RT for 18 h. The catalyst is filtered off through kieselguhr and the filtrate is concentrated under reduced pressure. After drying under high vacuum, this gives 10.7 g (95.9% of theory) of the target compound.

MS (DCI): m/z=199 (M+NH₄)⁺, 182 (M+H)⁺

¹H-NMR (400 MHz, CDCl₃): δ=7.10-7.02 (m, 1H), 6.35-6.23 (m, 2H), 4.58 (s, 2H), 3.79 (s, 3H), 3.65 (br. s, 2H).

### Example 29A

### Methyl {3-[(5-bromo-6-phenylfuro[2,3-d]pyrimidin-4-yl)amino]phenoxy}acetate

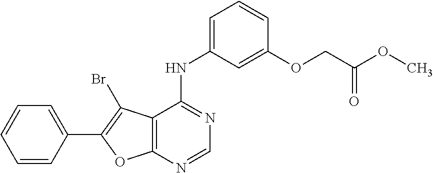

In a microwave, 657 mg (2.12 mmol) of 5-bromo-4-chloro-6-phenylfuro[2,3-d]pyrimidine, 500 mg (2.76 mmol) of methyl 3-aminophenoxyacetate and 0.70 ml (4.25 mmol) of diisopropylethylamine are heated at 180° C. for 30 min. After cooling, water is added and the reaction mixture is extracted repeatedly with ethyl acetate. The combined organic phases are washed with sat. sodium chloride solution, dried with sodium sulfate and concentrated under reduced pressure. From the crude product, 261 mg of the title compound (27.1% of theory) are isolated by chromatography on silica gel (cyclohexane/ethyl acetate 6:1).

LC-MS (method 7): Rₜ=2.82 min; m/z=454 (M+H)⁺.

¹H-NMR (400 MHz, DMSO-d₆): δ=8.59 (s, 1H), 8.52 (s, 1H), 8.08 (d, 2H), 7.65-7.48 (m, 4H), 7.35-7.28 (m, 2H), 6.23 (m, 1H), 4.82 (s, 2H), 3.74 (s, 3H).

### Example 30A

### 1-[(Z)-2-Chloro-2-nitroethenyl]-4-ethylbenzene

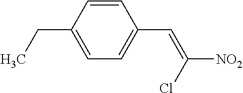

5.89 g (101.45 mmol) of potassium fluoride, 468.78 g (5.748 mol) of dimethylammonium chloride and 92.6 g (676.32 mmol) of 4-ethylbenzaldehyde are added to a solution of 115.67 g (743.95 mmol) of bromonitromethane in 2.78 liter of xylene. On a water separator, the mixture is vigorously stirred at reflux for 7 h. After cooling, 4.0 liter of water are added, the phases are separated and the aqueous phase is extracted twice with ethyl acetate. The combined organic phases are washed with saturated aqueous sodium chloride solution, dried over sodium sulfate and concentrated under reduced pressure. The residue is purified by chromatography on silica gel (cyclohexane/dichloromethane 1:1). This gives 108 g (75.5% of theory) of the title compound.

¹H-NMR (400 MHz, DMSO-d₆): δ=8.60 (s, 1H), 7.94 (d, 2H), 7.43 (d, 2H), 2.68 (q, 2H), 1.22 (t, 3H).

### Example 31A

### 5-(4-Ethylphenyl)furo[2,3-d]pyrimidin-4(3H)-one

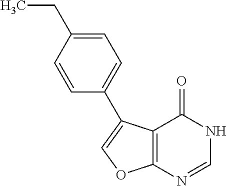

A mixture of 7.50 g (35.44 mmol) of 1-[(Z)-2-chloro-2-nitroethenyl]-4-ethylbenzene and 4.97 g (44.30 mmol) of 4,6-dihydroxypyrimidine in 130 ml of isopropanol is heated at reflux. After 10 min, 13.22 ml (88.59 mmol) of 1,8-diazabicyclo[5.4.0]undecan-7-ene (DBU) are added dropwise, and the resulting reaction mixture is stirred under reflux for 4 h and then, after cooling, concentrated under reduced pressure. The residue is taken up in ethyl acetate and water. The phases are separated and the organic phase is washed with water and saturated aqueous sodium chloride solution, dried over sodium sulfate and concentrated under reduced pressure. The residue is purified by chromatography on silica gel (cyclohexane/ethyl acetate 1:2). This gives 2.8 g (32.9% of theory) of the title compound.

HPLC (method 1): Rₜ=4.14 min.

MS (DCI): m/z=241 (M+H)⁺.

¹H-NMR (400 MHz, DMSO-d₆): δ=12.68 (s, 1H), 8.19 (s, 1H), 8.17 (d, 1H), 7.88 (d, 2H), 7.28 (d, 2H), 2.63 (q, 2H), 1.22 (t, 3H).

### Example 32A

### 4-Chloro-5-(4-ethylphenyl)furo[2,3-d]pyrimidine

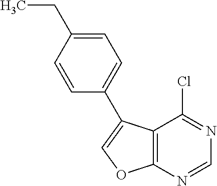

2.70 g (11.24 mmol) of 5-(4-ethylphenyl)furo[2,3-d]pyrimidin-4(3H)-one are mixed with 13.5 ml of sulfolane and 2.1 ml (22.48 mmol) of phosphorus oxychloride and heated to 120° C. After 1 h, the mixture is cooled and, with vigorous stirring, added dropwise to a mixture of 30 g of ice and 20 ml of conc. ammonia solution. The precipitated solid is filtered off with suction and washed well with water. The crude product is dissolved in 20 ml of dichloromethane and purified by chromatography on silica gel (cyclohexane/ethyl acetate 1:1). This gives 2.30 g (79.1% of theory) of the title compound.

HPLC (method 13): Rₜ=4.91 min.

MS (DCI): m/z=259 (M+H)⁺.

¹H-NMR (400 MHz, DMSO-d₆): δ=8.89 (s, 1H), 8.43 (s, 1H), 7.51 (d, 2H), 7.35 (d, 2H), 2.68 (q, 2H), 1.24 (t, 3H).

### Example 33A

Methyl(+)-(2S)-2-methyl-3-(trityloxy)propanoate

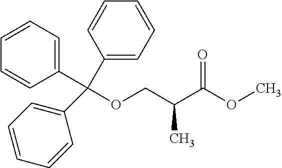

10.33 g (87.5 mmol) of (+)-methyl L-β-hydroxyisobutyrate are initially charged in 10 ml of dichloromethane and 14.2 ml (174.9 mmol) of pyridine and cooled to 0° C., and 1.07 g (8.7 mmol) of DMAP and, with ice cooling, 30.5 g (109 mmol) of triphenylmethyl chloride, dissolved in dichloromethane, are added. Cooling is removed and the mixture is stirred for 5 h and then, after dilution with dichloromethane, washed successively with water, 1 N hydrochloric acid, with saturated aqueous sodium bicarbonate solution and saturated aqueous sodium chloride solution. The organic phase is dried over sodium sulfate and concentrated under reduced pressure. The precipitated crystals are triturated with methanol, filtered off and then dried under reduced pressure. This gives 25.36 g of the target product (41.4% of theory).

MS (DCI): m/z=378 (M+NH₄)⁺

[α]_{D}²⁰=+6.4°, c=0.555, CHCl₃.

### Example 34A

### (+)-(2R)-2-Methyl-3-(trityloxy)propan-1-ol

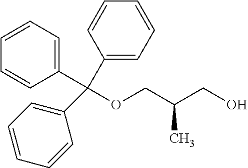

23 g (63.8 mmol) of methyl(+)-(2S)-2-methyl-3-(trityloxy)propanoate are dissolved in 100 ml of abs. THF and cooled to −20° C., and 31.9 ml (1 mol/l, 31.9 mmol) of lithium aluminum hydride solution in THF are added dropwise. After the addition has ended, the mixture is stirred at −10° C. for 10 min and then diluted with dichloromethane, and at about 0° C. saturated aqueous ammonium chloride solution is added carefully. The organic phase is washed with saturated aqueous sodium chloride solution, dried over sodium sulfate and concentrated under reduced pressure. The product is purified by chromatography (cyclohexane/ethyl acetate 5:1) on silica gel. This gives 11.16 g of the target compound (52.6% of theory).

¹H-NMR (400 MHz, DMSO-d₆): δ=7.40-7.25 (m, 15H), 4.39 (t, 1H), 3.43-3.38 (m, 1H), 3.32-3.28 (m, 1H), 3.02 (dd, 1H), 2.82 (dd, 1H), 1.84 (m, 1H), 0.88 (d, 3H).

[α]_{D}²⁰=+25.1°, c=0.575, CHCl₃.

### Example 35A

### Ethyl(−)-{[(2R)-2-methyl-3-(trityloxy)propyl]oxy}acetate

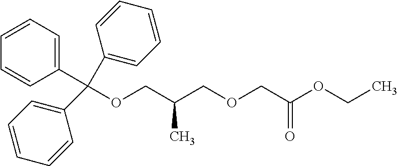

At 0° C., 3.4 ml (33.1 mmol) of ethyl diazoacetate are added dropwise with vigorous stirring to a suspension of 5.0 g (15.0 mmol) of (+)-(2R)-2-methyl-3-(trityloxy)propan-1-ol and 0.332 g (0.75 mmol, Rh₂OAc₄) of rhodium(II) acetate dimer in 25 ml of dry dichloromethane. After the addition has ended, stirring is continued at 0° C. for 5 min and the mixture is then warmed to RT and stirred at RT for a further 2.5 h. After dilution with dichloromethane, the mixture is washed with water and saturated aqueous sodium chloride solution, dried over sodium sulfate and concentrated under reduced pressure. The crude product is purified by chromatography on silica gel (cyclohexane/ethyl acetate 20:1). This gives 5.18 g of the target compound (79.7% of theory).

MS (DCI): m/z=436 (M+NH₄)⁺

¹H-NMR (400 MHz, DMSO-d₆): δ=7.40-7.25 (m, 15H), 4.10 (qu, 2H), 4.03 (s, 2H), 3.48 (dd, 1H), 3.38 (dd, 1H), 2.98 (dd, 1H), 2.40 (dd, 1H), 1.98 (m, 1H), 1.18 (t, 3H), 0.90 (d, 3H).

[α]_{D}²⁰=−0.9°, c=0.47, CHCl₃.

### Example 36A

### Ethyl(−)-{[(2S)-3-hydroxy-2-methylpropyl]oxy}acetate

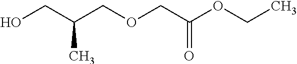

2.75 g (6.58 mmol) of ethyl(−)-{[(2R)-2-methyl-3-(trityloxy)propyl]oxy}acetate are dissolved in 25 ml of ethanol, 300 mg of 10% Pd/C are added and the mixture is stirred at RT under an atmosphere of hydrogen (atmospheric pressure) for 3 h. The mixture is filtered through Celite and the filtrate is concentrated under reduced pressure. The crude product is purified by filtration on silica gel (gradient cyclohexane/ethyl acetate 7:1 to 4:1). This gives 1.05 g of the target compound (90.6% of theory).

¹H-NMR (400 MHz, DMSO-d₆): δ=4.40 (t, 1H), 4.12 (qu, 2H), 4.05 (s, 2H), 3.41 (dd, 1H), 3.38-3.32 (m, 1H), 3.30-3.23 (m, 2H), 1.78 (m, 1H), 1.20 (t, 3H), 0.85 (d, 3H).

[α]_{D}²⁰=−11.9°, c=0.45, chloroform.

### Example 37A

### tert-Butyl {[(2R)-2-methyl-3-(trityloxy)propyl]oxy}acetate

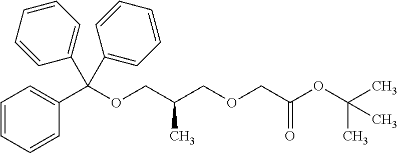

9.40 g (22.28 mmol) of (+)-(2R)-2-methyl-3-(trityloxy)propan-1-ol are dissolved in 28 ml of dichloromethane, and 625 mg (1.41 mmol) of rhodium diacetate dimer are added. The suspension is cooled to 0° C., and 6.5 ml (42.42 mmol) of tert-butyl diazoacetate are added dropwise with vigorous stirring. After the addition has ended, cooling is removed and the mixture is warmed to RT, stirred at RT for 2 h and then diluted with dichloromethane. The reaction mixture is washed three times with water and once with saturated aqueous sodium bicarbonate solution, dried over magnesium sulfate and concentrated under reduced pressure. The crude product is purified by chromatography on silica gel (gradient cyclohexane/dichloromethane 2:1 to 1:2). This gives 9.3 g of the target product (73.6% of theory).

¹H-NMR (400 MHz, DMSO-d₆): δ=7.40-7.24 (m, 15H), 3.89 (s, 2H), 3.46 (m, 1H) 3.35 (m, 1H), 2.98 (m, 1H), 2.89 (m, 1H), 1.97 (m, 1H), 1.41 (s, 9H), 0.90 (d, 3H).

### Example 38A

### tert-Butyl(−)-{[(2S)-3-hydroxy-2-methylpropyl]oxy}acetate

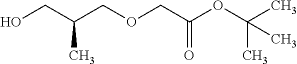

1.00 g (2.24 mmol) of tert-butyl {[(2R)-2-methyl-3-(trityloxy)propyl]oxy}acetate are dissolved in 5 ml of ethanol, and 238.3 mg of palladium on carbon (10% Pd/C) are added. Under an atmosphere of hydrogen (atmospheric pressure) the suspension is stirred vigorously at RT overnight. The reaction mixture is filtered off through kieselguhr and the filtrate obtained is concentrated under reduced pressure. The crude product is purified by chromatography on silica gel (gradient cyclohexane/ethyl acetate 10:1 to 1:1). This gives 350 mg of the target product (76.5% of theory).

¹H-NMR (400 MHz, DMSO-d₆): δ=4.39 (t, 1H), 3.93 (s, 2H), 3.41-3.22 (m, 4H), 1.78 (m, 1H), 1.43 (s, 9H), 0.84 (d, 3H).

[α]_{D}²⁰=−13.0°, c=0.495, CHCl₃.

### Example 39A

### tert-Butyl 3-[(1S)-2-benzyloxy-1-methylethoxy]propanoate

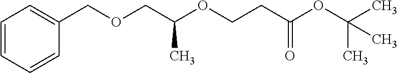

A mixture of 20 g (120.3 mmol) of (+)-(S)-1-benzyloxy-2-propanol and 123 g (962 mmol) of tert-butyl acrylate is cooled to 0° C., and 962 mg (60% strength, 24 mmol) of sodium hydride are added in a plurality of portions. The mixture is stirred at 0° C. for 10 min, and saturated aqueous ammonium chloride solution is then added carefully. After phase separation, the aqueous phase is extracted twice with dichloromethane. The organic phases are combined, dried over magnesium sulfate and concentrated under reduced pressure and then under high vacuum. The crude product is purified by chromatography on silica gel (cyclohexane/ethyl acetate 30:1). This gives 18.4 g of the target compound (51.9% of theory).

¹H-NMR (400 MHz, DMSO-d₆): δ=7.38-7.25 (m, 5H), 4.49 (s, 2H), 3.64 (t, 2H), 3.61-3.58 (m, 1H), 3.40 (dd, 1H), 3.32 (dd, 1H), 2.39 (t, 2H), 1.39 (s, 9H), 1.05 (d, 3H).

### Example 40A

### tert-Butyl(+)-3-[(1S)-2-hydroxy-1-methylethoxy]propanoate

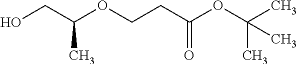

18.1 g (61.5 mmol) of ethyl {[(2R)-2-methyl-3-(trityloxy)propyl]oxy}acetate are dissolved in 100 ml of ethanol, 1.96 g of 10% Pd/C are added and the mixture is stirred at RT under an atmosphere of hydrogen (atmospheric pressure) for 2 h. The mixture is filtered through Celite and the filtrate is concentrated under reduced pressure. This gives 13.8 g of the target compound as a crude product which is not purified any further (about 92% of theory).

MS (DCI): m/z=222 (M+NH₄)⁺

¹H-NMR (400 MHz, DMSO-d₆): δ=4.50 (t, 1H), 3.67-3.60 (m, 2H), 3.40-3.34 (m, about 2H), 3.27-3.21 (m, 1H), 2.39 (t, 2H), 1.39 (s, 9H), 1.02 (d, 3H).

[α]_{D}²⁰=+15.0°, c=0.490, chloroform.

### Example 41A

### Ethyl(−)-{[(2R)-3-{[5-(4-methoxyphenyl)furo[2,3-d]pyrimidin-4-yl]oxy}-2-methylpropyl]oxy}-acetate

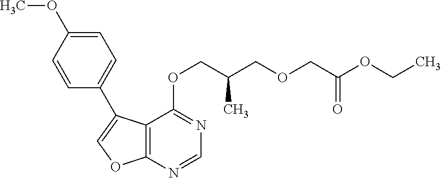

In four portions, 168 mg of sodium hydride are added to a mixture, cooled to 0° C., of 1000 mg (3.84 mmol) of 4-chloro-5-(4-methoxyphenyl)furo[2,3-d]pyrimidine and 946 mg (5.37 mmol) of ethyl(−)-{[(2S)-3-hydroxy-2-methylpropyl]oxy}acetate in 5.0 ml of DMF. The reaction mixture is stirred at 0° C. for 1 h and at RT overnight and then added to water. The mixture is extracted three times with ethyl acetate and the organic phases are combined. The organic phases are washed with saturated aqueous ammonium chloride solution and saturated aqueous sodium chloride solution, dried over magnesium sulfate and concentrated under reduced pressure. The residue is pre-purified by chromatography on silica gel (gradient cyclohexane/ethyl acetate 10:1 to 3:1). Further purification is by preparative RP-HPLC (gradient acetonitrile/water). This gives 248 mg (16.1% of theory) of the title compound.

LC-MS (method 14): Rₜ=1.33 min.; m/z=401 (M+H)⁺.

¹H-NMR (400 MHz, DMSO-d₆): δ=8.59 (s, 1H), 8.26 (s, 1H), 7.78 (d, 2H), 7.02 (d, 2H), 4.51 (dd, 1H), 4.41 (dd, 1H), 4.11-4.04 (m, 4H), 3.82 (s, 3H), 3.48-3.41 (m, 2H), 2.21 (m, 1H), 1.16 (t, 3H), 0.96 (d, 3H).

[α]_{D}²⁰=−7.7°, c=0.485, cloroform.

The following examples are obtained by an analogous procedure:

### Example 42A

### tert-Butyl(+)-3-[(1S)-2-{[5-(4-methoxyphenyl)furo[2,3-d]pyrimidin-4-yl]oxy}-1-methylethoxy]propanoate

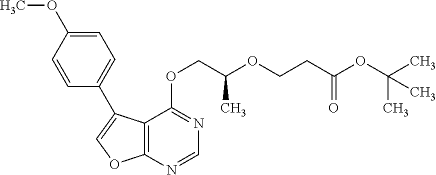

Starting with 4-chloro-5-(4-methoxyphenyl)furo[2,3-d]pyrimidine and tert-butyl 3-[(1S)-2-hydroxy-1-methylethoxy]propanoate, 530 mg (32.2% of theory) of the title compound are obtained.

LC-MS (method 7): Rₜ=2.80 min.; m/z=429 (M+H)⁺.

¹H-NMR (400 MHz, DMSO-d₆): δ=8.60 (s, 1H), 8.28 (s, 1H), 7.72 (d, 2H), 7.02 (d, 2H), 4.53-4.41 (m, 2H), 3.87-3.80 (m, 1H), 3.81 (s, 3H), 3.70-3.58 (m, 2H), 2.37 (t, 2H), 1.31 (s, 9H), 1.15 (d, 3H). P [α]_{D}²⁰=+15.0°, c=0.490, chloroform.

### Example 43A

### tert-Butyl(−)-(6R)-6-{[5-(4-ethyl phenyl)furo[2,3-d]pyrimidin-4-yl]oxy}heptanoate

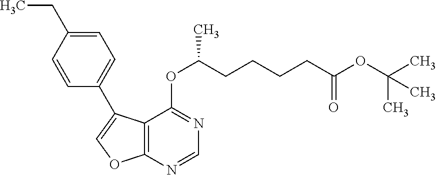

Starting with 4-chloro-5-(4-ethylphenyl)furo[2,3-d]pyrimidine and tert-butyl (6R)-6-hydroxyheptanoate, 363 mg (44.2% of theory) of the title compound are obtained.

LC-MS (method 6): Rₜ=2.93 min.; m/z=425 (M+H)⁺.

¹H-NMR (400 MHz, DMSO-d₆): δ=8.58 (s, 1H), 8.29 (s, 1H), 7.67 (d, 2H), 7.31 (d, 2H), 5.47 (m, 1H), 2.67 (q, 2H), 2.13 (t, 2H), 1.75-1.65 (m, 2H), 1.51-1.45 (m, 2H), 1.40-1.26 (m, including s, together 14H), 1.25 (t, 3H).

[α]_{D}²⁰=−47.6°, c=0.550, chloroform.

### Example 44A

### tert-Butyl(−)-{[(2R)-3-{[5-(4-ethylphenyl)furo[2,3-d]pyrimidin-4-yl]oxy}-2-methylpropyl]oxy}acetate

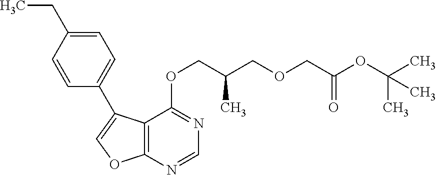

Starting with 4-chloro-5-(4-ethylphenyl)furo[2,3-d]pyrimidine and tert-butyl(−)-{[(2S)-3-hydroxy-2-methylpropyl]oxy}acetate, 454 mg (55.1% of theory) of the title compound are obtained.

LC-MS (method 7): Rₜ=3.12 min.; m/z=427 (M+H)⁺.

¹H-NMR (400 MHz, DMSO-d₆): δ=8.60 (s, 1H), 8.30 (s, 1H), 7.65 (d, 2H), 7.29 (d, 2H), 4.49 (dd, 1H), 4.41 (dd, 1H), 3.91 (s, 2H), 3.45-3.38 (m, 2H), 2.67 (q, 2H), 2.20 (m, 1H), 1.39 (s, 9H), 1.21 (t, 3H), 0.95 (d, 3H).

[α]_{D}²⁰=−4.0°, c=0.480, chloroform.

### Example 45A

### tert-Butyl(+)-3-[(1S)-2-{[5-(4-ethylphenyl)furo[2,3-d]pyrimidin-4-yl]oxy}-1-methylethoxy]propanoate

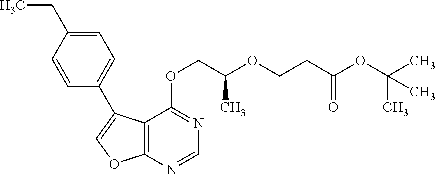

Starting with 4-chloro-5-(4-ethylphenyl)furo[2,3-d]pyrimidine and tert-butyl 3-[(1S)-2-hydroxy-1-methylethoxy]propanoate, 158 mg (19.2% of theory) of the title compound are obtained.

LC-MS (method 7): Rₜ=3.08 min.; m/z=427 (M+H)⁺.

¹H-NMR (400 MHz, DMSO-d₆): δ=8.61 (s, 1H), 8.31 (s, 1H), 7.70 (d, 2H), 7.29 (d, 2H), 4.51 (dd, 1H), 4.43 (dd, 1H), 3.82 (m, 1H), 3.69-3.54 (m, 2H), 2.64 (q, 2H), 2.35 (t, 2H), 1.33 (s, 9H), 1.22 (t, 3H), 1.15 (d, 3H).

[α]_{D}²⁰=+14.2°, c=0.445, chloroform.

### Example 46A

### tert-butyl(+)-3-{(1S)-2-[(5-bromo-6-phenylfuro[2,3-d]pyrimidin-4-yl)oxy]-1-methylethoxy}propanoate

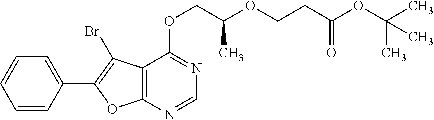

Starting with 5-bromo-4-chloro-6-phenylfuro[2,3-d]pyrimidine and tert-butyl 3-[(1S)-2-hydroxy-1-methylethoxy]propanoate, 911 mg (59.1% of theory) of the title compound are obtained.

LC-MS (method 6): Rₜ=2.75 min.; m/z=477 (M+H)⁺.

¹H-NMR (400 MHz, DMSO-d₆): δ=8.62 (s, 1H), 8.07 (d, 2H), 7.64-7.53 (m, 3H), 4.58 (dd, 1H), 4.48 (dd, 1H), 3.92-3.88 (m, 1H), 3.80-3.72 (m, 2H), 2.41 (t, 2H), 1.34 (s, 9H), 1.28 (d, 3H).

[α]_{D}²⁰=+9.0°, c=0.485, chloroform.

### Example 47A

### Ethyl(−)-{(2R)-3-[(5-bromo-6-phenylfuro[2,3-d]pyrimidin-4-yl)oxy]-2-methylpropyl}oxy)acetate

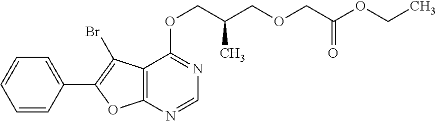

A mixture of 1000 mg (3.23 mmol) of 5-bromo-4-chloro-6-phenylfuro[2,3-d]pyrimidine and 797 mg (4.52 mmol) of ethyl(−)-{[(2S)-3-hydroxy-2-methylpropyl]oxy}acetate in 6 ml of DMF and 6 ml of THF is cooled to −10°, and 3.55 ml (3.55 mmol) of a 1 N solution of phosphazene base in hexane are added dropwise. After the end of the addition, the mixture is stirred at −10° C. for 1 h and then added to water. The mixture is extracted three times with ethyl acetate, the organic phases are combined and the combined organic phase is washed with saturated aqueous sodium chloride solution, dried over magnesium sulfate and concentrated under reduced pressure. The residue is purified by chromatography on silica gel (gradient cyclohexane/ethyl acetate 10:1 to 5:1). Further purification is by preparative RP-HPLC (gradient acetonitrile/water). This gives 225 mg (15.5% of theory) of the title compound.

LC-MS (method 14): R=1.54 min.; m/z=449/451 (M+H)⁺.

¹H-NMR (400 MHz, DMSO-d₆): δ=8.62 (s, 1H), 8.09 (d, 2H), 7.63-7.53 (m, 3H), 4.55 (dd, 1H), 4.45 (dd, 1H), 4.15-4.06 (m, 4H), 3.64-3.55 (m, 2H), 2.30 (m, 1H), 1.18 (t, 3H), 1.11 (d, 3H).

[α]_{D}²⁰=−6.7°, c=0.50, chloroform.

### Example 48A

### tert-Butyl 3-[(1S)-2-{[6-bromo-5-(4-methoxyphenyl)furo[2,3-d]pyrimidin-4-yl]oxy}-1-methylethoxy]-propanoate

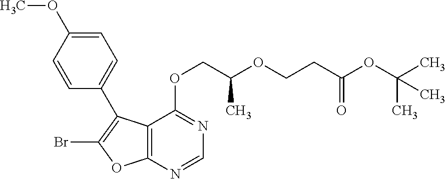

182.8 mg (1.03 mmol) of N-bromosuccinimide are added to a mixture of 418 mg (0.93 mmol) of tert-butyl(+)-3-[(1S)-2-{[5-(4-methoxyphenyl)furo[2,3-d]pyrimidin-4-yl]oxy}-1-methylethoxy]propanoate in 2.0 ml of acetonitrile. After about 20 min of stirring at RT, the mixture is separated by preparative RP-HPLC (gradient acetonitrile/water). This gives 418 mg (88.3% of theory) of the title compound.

LC-MS (method 14): Rₜ=1.57 min.; m/z=507/509 (M+H)⁺.

¹H-NMR (400 MHz, DMSO-d₆): δ=8.59 (s, 1H), 7.55 (d, 2H), 7.05 (d, 2H), 4.44-4.33 (m, 2H), 3.83 (s, 3H), 3.73-3.68 (m, 1H), 3.58-3.45 (m, 2H), 2.39 (t, 2H), 1.33 (s, 9H), 1.04 (d, 3H).

[α]_{D}²⁰=+18.4°, c=0.50, chloroform.

The following examples are obtained in an analogous manner by bromination of appropriate starting materials with N-bromosuccinimide:

| Example | Structure | Analytical data |
|---|---|---|
| 49A | 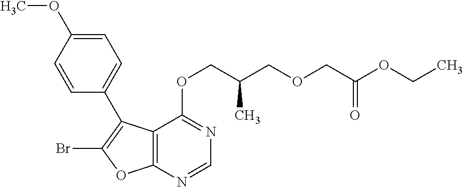 | LC-MS (method 14): Rₜ = 1.46 min; m/z = 479/481 (M + H)⁺. ¹H-NMR (400 MHz, DMSO-d₆): δ = 8.59 (s, 1H), 7.53 (d, 2H), 7.06 (d, 2H), 4.41 (dd, 1H), 4.34 (dd, 1H), 4.09 (q, 2H), 4.01 (s, 2H), 3.82 (s, 3H), 3.35-3.28 (m, about 2H), 2.10 (m, 1H), 1.18 (t, 3H), 0.85 (d, 3H). |
| 50A | 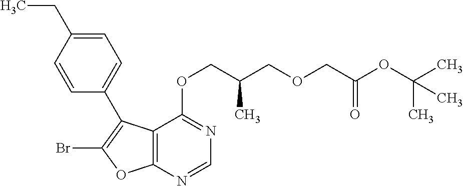 | LC-MS (method 7): Rₜ = 3.28 min; m/z = 449/451 (M + H)⁺. ¹H-NMR (400 MHz, DMSO-d₆): δ = 8.59 (s, 1H), 7.51 (d, 2H), 7.32 (d, 2H), 4.40-4.31 (m, 2H), 3.87 (s, 2H), 3.26 (s, 2H), 2.68 (q, 2H), 2.05 (m, 1H), 1.39 (s, 9H), 1.23 (t, 3H), 0.71 (d, 3H). [α]_{D}²⁰ = −5.9°, c = 0.485, chloroform. |
| 51A | 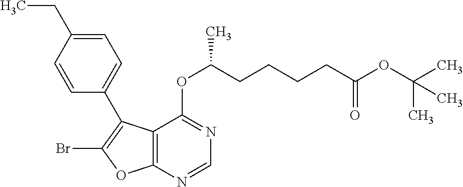 | LC-MS (method 7): Rₜ = 3.44 min; m/z = 503/505 (M + H)⁺. ¹H-NMR (400 MHz, DMSO-d₆): δ = 8.68 (s, 1H), 7.50 (d, 2H), 7.33 (d, 2H), 5.34 (m, 1H), 2.69 (q, 2H), 2.09 (t, 2H), 1.59-1.51 (m, 2H), 1.45-1.55 (m, 2H), 1.36 (s, 9H), 1.29-1.17 (m, about 2H). [α]_{D}²⁰ = −51.8°, c = 0.450, chloroform. |
| 52A | 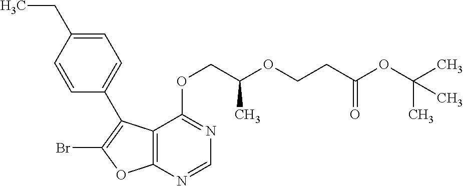 | LC-MS (method 14): Rₜ = 1.72 min; m/z = 505/507 (M + H)⁺. ¹H-NMR (400 MHz, DMSO-d₆): δ = 8.60 (s, 1H), 7.52 (d, 2H), 7.32 (d, 2H), 4.42 (dd, 1H), 4.34 (dd, 1H), 3.68 (m, 1H), 3.55-3.42 (m, 2H), 2.68 (q, 2H), 2.27 (t, 2H), 1.32 (s, 9H), 1.22 (t, 3H), 1.03 (d, 3H). [α]_{D}²⁰ = +28.8°, c = 0.430, chloroform. |

### WORKING EXAMPLES

### Example 1

### Methyl 6-{[5-(5-methylpyridin-2-yl)-6-phenylfuro[2,3-d]pyrimidin-4-yl]amino}hexanoate

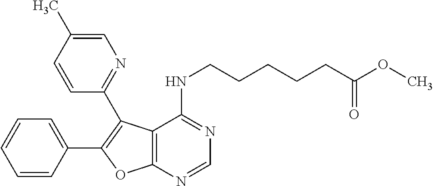

Under argon, 150 mg (0.32 mmol) of methyl 6-{[6-phenyl-5-(4,4,5,5-tetramethyl-1,3,2-dioxaborolan-2-yl)furo[2,3-d]pyrimidin-4-yl]amino}hexanoate are dissolved in 600 μl of DMSO, and 11 mg (0.02 mmol) of bis(triphenylphosphine)palladium(II) chloride, 67 mg (0.48 mmol) of potassium carbonate, 60 μl of methanol (10% by volume) and 61 mg (0.36 mmol) of 2-bromo-5-methylpyridine are added. The mixture is stirred at 80° C. for 3 h. The reaction mixture is diluted with DMSO and the crude product is purified by preparative RP-HPLC (gradient: water/acetonitrile). This gives 41 mg (29% of theory) of the title compound.

LC-MS (method 7): Rₜ=2.88 min.; m/z=431 (M+H)⁺.

¹H-NMR (400 MHz, DMSO-d₆): δ=9.28 (t, 1H), 8.61 (s, 1H), 8.29 (s, 1H), 7.63-7.56 (m, 3H), 7.52-7.46 (m, 3H), 7.28 (d, 1 h), 3.57 (s, 3H), 3.51 (q, 2H), 2.35 (s, 3H), 2.32 (t, 2H), 1.67-1.54 (m, 4H), 1.42-1.32 (m, 2H).

### Example 2

### Methyl 6-{[5-(5-formylpyridin-2-yl)-6-phenylfuro[2,3-d]pyrimidin-4-yl]amino}hexanoate

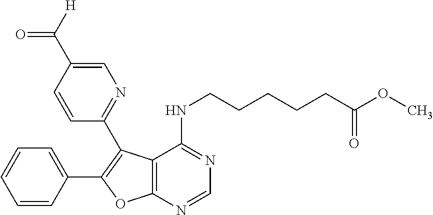

Under argon, 150 mg (0.32 mmol) of methyl 6-{[6-phenyl-5-(4,4,5,5-tetramethyl-1,3,2-dioxaborolan-2-yl)furo[2,3-d]pyrimidin-4-yl]amino}hexanoate are dissolved in 0.6 ml of DMSO, 11 mg (0.02 mmol) of bis(triphenylphosphine)palladium(II) chloride, 67 mg (0.48 mmol) of potassium carbonate, 60 μl of methanol (10% by volume) and 66 mg (0.36 mmol) of 6-bromopyridine-3-carbaldehyde are added. The mixture is stirred at 80° C. for 3 h. The reaction mixture is diluted with DMSO, and 70 mg of the title compound (49% of theory) are isolated from the crude product by preparative RP-HPLC (gradient of water and acetonitrile).

LC-MS (method 3): Rₜ=2.45 min; m/z=445 (M+H)⁺.

¹H-NMR (400 MHz, DMSO-d₆): δ=10.12 (s, 1H), 9.28 (d, 1H), 9.06 (t, 1H), 8.33 (s, 1H), 8.17 (dd, 1H), 7.63 (dd, 2H), 7.57 (s, 1H), 7.53 (t, 3H), 3.56 (s, 3H), 3.53 (t, 2H), 2.32 (t, 2H), 1.69-1.55 (m, 4H), 1.43-1.33 (m, 2H).

### Example 3

### Methyl 6-{[6-phenyl-5-(5-vinylpyridin-2-yl)furo[2,3-d]pyrimidin-4-yl]amino}hexanoate

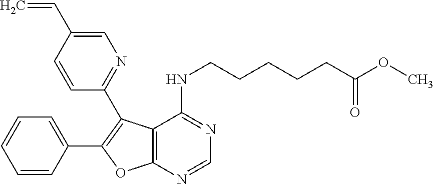

Under argon, 63 mg (0.14 mmol) of methyl 6-{[5-(5-formylpyridin-2-yl)-6-phenylfuro[2,3-d]pyrimidin-4-yl]amino}hexanoate are dissolved in 0.2 ml of dichloromethane, and 61 mg (0.17 mmol) of methyl(triphenyl)phosphonium bromide and 27 mg (0.18 mmol) of 1,8-diazabicyclo[5.4.0]undec-7-ene are added. The mixture is stirred at R_{T} for 4 h. The reaction mixture is diluted with dichloromethane and washed with saturated sodium bicarbonate solution and saturated sodium chloride solution, and the organic phase is dried over sodium sulfate and concentrated on a rotary evaporator. Drying under high vacuum gives 21 mg (33% of theory) of the title compound.

LC-MS (method 2): Rₜ=2.96 min; m/z=443 (M+H)⁺.

¹H-NMR (400 MHz, DMSO-d₆): δ=9.26 (t, 1H), 8.82 (d, 1H), 8.30 (s, 1H), 7.94 (dd, 1H), 7.61 (q, 2H), 7.51 (m, 3H), 7.34 (d, 1H), 6.82 (q, 1H), 6.04 (d, 1H), 5.48 (d, 1H), 3.56 (s, 3H), 3.52 (q, 2H), 2.31 (t, 2H), 1.61 (m, 4H), 1.37 (m, 2H).

### Example 4

### Methyl 6-{[5-(5-ethylpyridin-2-yl)-6-phenylfuro[2,3-d]pyrimidin-4-yl]amino}hexanoate

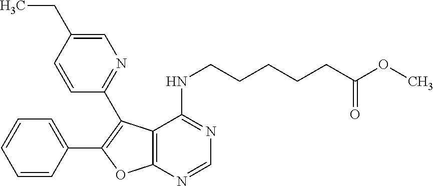

0.5 mg of 10% palladium on carbon are initially charged under argon, and 19 mg (0.04 mmol) of methyl 6-{[6-phenyl-5-(5-vinyl pyridin-2-yl)furo[2,3-d]pyrimidin-4-yl]amino}hexanoate dissolved in 0.3 ml of ethanol are added. The mixture is stirred under a hydrogen atmosphere at atmospheric pressure and RT for 4.5 h. The reaction is filtered through Celite. The filter cake is washed with ethanol, the filtrate is concentrated on a rotary evaporator and the residue is dried under reduced pressure. This gives 6 mg (29% of theory) of the title compound.

LC-MS (method 7): Rₜ=3.02 min; m/z=445 (M+H)⁺.

### Example 5

### tert-Butyl (6R)-6-{[5-(5-ethylpyridin-2-yl)-6-phenylfuro[2,3-d]pyrimidin-4-yl]oxy}heptanoate

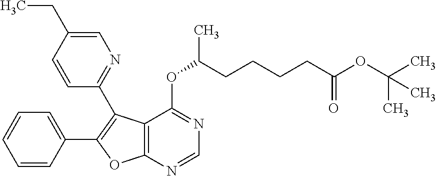

Under argon, 100 mg (0.21 mmol) of tert-butyl (6R)-6-[(5-bromo-6-phenylfuro[2,3-d]pyrimidin-4-yl)oxy]heptanoate are dissolved in 5 ml of toluene, and 100 mg (0.25 mmol) of 5-ethyl-2-(tributylstannyl)pyridine and 12 mg (0.01 mmol) of tetrakis(triphenyl-phosphine)palladium(0) are added. The reaction mixture is stirred at reflux for 12 h, and the reaction mixture is then filtered through Celite. The filtrate is washed with saturated sodium chloride solution and concentrated on a rotary evaporator. The residue is purified by preparative RP-HPLC (gradient: water/acetonitrile). This gives 23 mg (22% of theory) of the title compound.

LC-MS (method 7): Rₜ=3.38 min; m/z=502 (M+H)⁺.

¹H-NMR (400 MHz, DMSO-d₆): δ=8.59 (s, 1H), 8.53 (d, 1H), 7.77 (dd, 1H), 7.57 (m, 3H), 7.4 (m, 3H), 5.24 (q, 1H), 2.72 (q, 2H), 2.08 (q, 2H), 1.54-1.04 (m, 21H).

### Example 6

### tert-Butyl (6R)-6-[(5-cyclopent-1-en-1-yl-6-phenylfuro[2,3-d]pyrimidin-4-yl)oxy]heptanoate

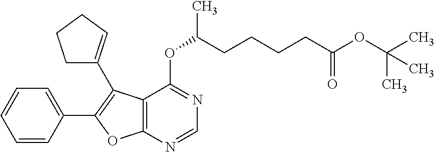

Under argon, 500 mg (1.05 mmol) of tert-butyl (6R)-6-[(5-bromo-6-phenylfuro[2,3-d]pyrimidin-4-yl)oxy]heptanoate are dissolved in 10 ml of THF, and 5.3 ml of a 2 M (10.52 mmol) sodium carbonate solution, 73 mg (0.11 mmol) of bis(triphenylphosphine)-palladium(II) chloride and 265 mg (2.37 mmol) of cyclopenten-1-ylboronic acid are added. The mixture is stirred at reflux for 12 h. The reaction mixture is filtered through Celite and the filtrate is concentrated. The crude product is purified by chromatography on silica gel (cyclohexane/ethyl acetate 5:1). This gives 548 mg of the title compound (58% of theory).

LC-MS (method 5): Rₜ=5.12 min; m/z=463 (M+H)⁺.

¹H-NMR (400 MHz, DMSO-d₆): δ=8.54 (s, 1H), 7.76 (d, 2H), 7.5 (t, 2H), 7.44 (q, 1H), 5.94 (t, 1H), 5.4 (q, 1H), 2.63 (m, 2H), 2.16 (t, 2H), 2.03 (m, 2H), 1.68 (q, 2H), 1.5 (m, 2H), 1.34 (m, 16H).

[α]_{D}²⁰=−50, c=0.340 chloroform.

### Example 7

### tert-Butyl (6R)-6-{[5-(4-methoxyphenyl)-6-pyridin-2-ylfuro[2,3-d]pyrimidin-4-yl]oxy}heptanoate

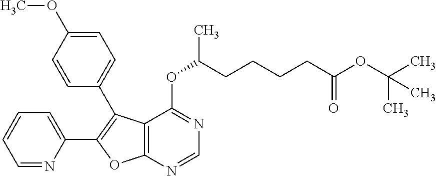

Under argon, 165 mg (0.33 mmol) of tert-butyl (6R)-6-{[6-bromo-5-(4-methoxyphenyl)furo[2,3-d]pyrimidin-4-yl]oxy}heptanoate are dissolved in toluene, and 150 mg (0.40 mmol) of 2-tri-n-butylstannylpyridine and 19 mg (0.02 mmol) of tetrakis(triphenylphosphine)palladium are added. The reaction mixture is heated at reflux for 12 h. The crude product is purified directly by preparative RP-HPLC (gradient: water/acetonitrile). This gives 160 mg of the title compound (97% of theory).

LC-MS (method 5): Rₜ=4.38 min; m/z=504 (M+H)⁺.

¹H-NMR (400 MHz, DMSO-d₆): δ=8.61 (s, 1H), 8.57 (d, 1H), 7.83 (t, 1H), 7.57 (d, 1H), 7.42 (d, 2H), 7.37 (q, 1H), 6.97 (d, 2H), 5.3 (m, 1H), 3.81 (s, 3H), 2.1 (t, 2H), 1.5 (m, 2H), 1.39 (m, 2H), 1.34 (s, 9H), 1.24 (d, 3H), 1.15 (m, 2H).

[α]_{D}²⁰=−69°, c=0.455, chloroform.

### Example 8

### tert-Butyl (6R)-6-[(5-cyclopent-1-en-1-yl-6-phenylfuro[2,3-d]pyrimidin-4-yl)oxy]heptanoate

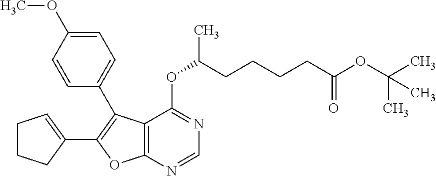

Under an atmosphere of argon, 210 mg (0.42 mmol) of tert-butyl (6R)-6-{[6-bromo-5-(4-methoxyphenyl)furo[2,3-d]pyrimidin-4-yl]oxy}heptanoate are initially charged in 4 ml of THF, and 2.1 ml (4.16 mmol) of a 2 M aqueous sodium carbonate solution, 29 mg (0.04 mmol) of bis(triphenylphosphine)palladium(II) chloride and 104 mg (0.94 mmol) of cyclopent-1-en-1-ylboronic acid are added in succession. The reaction mixture is stirred under reflux for 1.5 h. The catalyst is filtered off through Celite, the filter residue is washed with THF and the combined filtrate is concentrated under reduced pressure. The residue is purified by preparative RP-HPLC (gradient: water/acetonitrile). This gives 131 mg (64% of theory) of the target compound.

LC-MS (method 10): Rₜ=5.17 min; m/z=493 (M+H)⁺.

¹H-NMR (400 MHz, DMSO-d₆): δ=8.49 (s, 1H), 7.33 (d, 2H), 6.98 (d, 2H), 6.35 (m, 1H), 5.21 (m, 1H), 3.81 (s, 3H), 2.45 (m, 2H), 2.29 (m, 2H), 2.08 (t, 2H), 1.83 (m, 2H), 1.52-1.29 (m, 13H: including: 1.36 (s, 9H)), 1.22-0.98 (m, 5H: including 1.18 (d, 3H)).

[α]_{D}²⁰=−48°, c=0.590, chloroform.

### Example 9

### tert-Butyl (6R)-6-{[6-cyclopentyl-5-(4-methoxyphenyl)furo[2,3-d]pyrimidin-4-yl]oxy}heptanoate

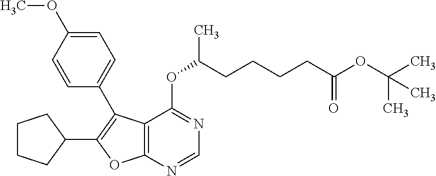

59 mg (0.12 mmol) of tert-butyl (6R)-6-[(5-cyclopent-1-en-1-yl-6-phenylfuro[2,3-d]pyrimidin-4-yl)oxy]heptanoate are initially charged in 10 ml of ethanol, 6 mg of 10% palladium on carbon are added and the mixture is stirred under a hydrogen atmosphere at atmospheric pressure and RT overnight. The catalyst is filtered off through Celite, the filter residue is washed with ethanol and the filtrate is concentrated under reduced pressure. The residue is purified by preparative RP-HPLC (gradient: water/acetonitrile). This gives 20 mg of the target compound (32% of theory).

LC-MS (method 10): Rₜ=5.14 min; m/z=495 (M+H)⁺.

¹H-NMR (400 MHz, CDCl₃): δ=8.42 (s, 1H), 7.35 (d, 2H), 6.95 (d, 2H), 5.35 (m, 1H), 3.87 (s, 3H), 3.25 (m, 1H), 2.12 (t, 2H), 1.95 (m, 4H), 1.89 (m, 2H), 1.70-1.48 (m, 6H), 1.41 (s, 9H), 1.35-1.18 (m, 5H: including 1.28 (d, 3H)).

[α]_{D}²⁰=−35°, c=0.490, chloroform.

### Example 10

### tert-Butyl (6R)-6-[(5-cyclohex-1-en-1-yl-6-phenylfuro[2,3-d]pyrimidin-4-yl)oxy]heptanoate

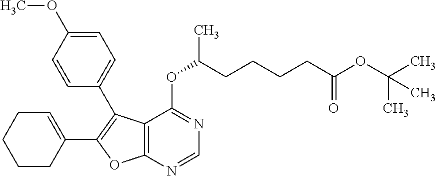

Under an atmosphere of argon, 268 mg (0.53 mmol) of tert-butyl (6R)-6-{[6-bromo-5-(4-methoxyphenyl)furo[2,3-d]pyrimidin-4-yl]oxy}heptanoate are dissolved in 1.5 ml of DMSO, and 0.5 ml (1.05 mmol) of a 2 M aqueous sodium carbonate solution, 22 mg (0.03 mmol) of bis(triphenylphosphine)palladium(II) chlorid and 166 mg (0.80 mmol) of 2-cyclohex-1-en-1-yl-4,4,5,5-tetramethyl-1,3,2-dioxaborolane are added in succession. The reaction mixture is stirred at 80° C. for 3 h. The reaction solution is diluted with DMSO and 193 mg of the target compound (72% of theory) are isolated from the residue by preparative RP-HPLC (gradient: water/acetonitrile).

LC-MS (method 10): R=5.23 min; m/z=507 (M+H)⁺.

¹H-NMR (400 MHz, DMSO-d6): δ=8.48 (s, 1H), 7.53 (d, 2H), 6.95 (d, 2H), 6.35 (m, 1H), 5.21 (m, 1H), 3.80 (s, 3H), 2.14 (m, 2H), 2.11-1.99 (m, 4H: including 2.09 (t, 2H)), 1.55 (m, 2H), 1.50-1.30 (m 13H: including 1.35 (s, 9H)), 1.18 (d, 3H), 1.15-0.99 (m, 2H).

[α]_{D}²⁰=−55°, c=0.545, chloroform.

### Example 11

### tert-Butyl (6R)-6-{[5-(4-ethyl cyclohex-1-en-1-yl)-6-phenylfuro[2,3-d]pyrimidin-4-yl]oxy}heptanoate

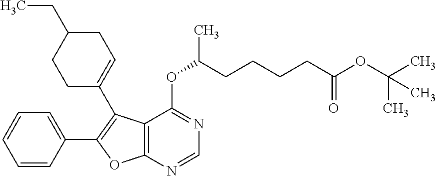

Under an atmosphere of argon, 250 mg (0.53 mmol) of tert-butyl (6R)-6-[(5-bromo-6-phenylfuro[2,3-d]pyrimidin-4-yl)oxy]heptanoate are initially charged in 2 ml of DMSO, and 0.5 ml (1.05 mmol) of 2 M aqueous sodium carbonate solution, 37 mg (0.05 mmol) of bis(triphenylphosphine)palladium(II) chloride and 186 mg (0.79 mmol) of 2-(4-ethylcyclohex-1-en-1-yl)-4,4,5,5-tetramethyl-1,3,2-dioxaborolane are added in succession. The reaction mixture is stirred at 80° C. overnight. The catalyst is filtered off through Celite, the filter residue is washed with dichloromethane, the filtrate is washed with water and saturated sodium chloride solution and the organic phase is concentrated under reduced pressure. The residue is purified by preparative RP-HPLC (gradient: water/acetonitrile). This gives 82 mg (31% of theory) of the target compound.

LC-MS (method 7): Rₜ=3.99 min; m/z=505 (M+H)⁺.

¹H-NMR (400 MHz, DMSO-d₆): δ=8.52 (s, 1H), 7.81 (d, 2H), 7.50 (t, 2H), 7.41 (t, 1H), 5.78 (m, 1H), 5.40 (m, 1H), 2.45-2.20 (m, 3H), 2.17 (m, 2H), 1.96-1.87 (m, 1H), 1.85-1.26 (m, 23H: including 1.35 (s, 9H)), 0.96 (t, 3H).

[α]_{D}²⁰=−63°, c=0.355, chloroform.

The following compounds are prepared in an analogous manner:

### Example 12

### tert-Butyl (6R)-6-{[5-(4-methylcyclohex-1-en-1-yl)-6-phenylfuro[2,3-d]pyrimidin-4-yl]oxy}heptanoate

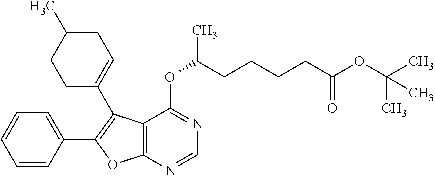

LC-MS (method 7): Rₜ=3.79 min; m/z=491 (M+H)⁺.

¹H-NMR (400 MHz, DMSO-d₆): δ=8.53 (s, 1H), 7.82 (d, 2H), 7.51 (t, 2H), 7.41 (t, 1H), 5.78 (m, 1H), 5.40 (m, 1H), 2.44-2.13 (m, 5H), 1.91-1.64 (m, 5H), 1.57-1.28 (m, 17H: including 1.34 (s, 9H)), 1.04 (d, 3H).

[α]_{D}²⁰=−62°, c=0.185, chloroform.

### Example 13

### tert-Butyl (6R)-6-{[5-(4-methoxycyclohex-1-en-1-yl)-6-phenylfuro[2,3-d]pyrimidin-4-yl]oxy}heptanoate

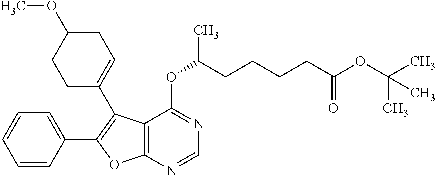

LC-MS (method 7): Rₜ=3.57 min.; m/z=507 (M+H)⁺.

¹H-NMR (400 MHz, DMSO-d₆): δ=8.53 (s, 1H), 7.82 (d, 2H), 7.49 (t, 2H), 7.42 (t, 1H), 5.68 (m, 1H), 5.40 (m, 1H), 3.63 (m, 1H), 3.36 (s, 3H), 2.40 (m, 2H), 2.23-1.95 (m, 3H), 1.82 (m, 1H), 1.71 (m, 2H), 1.52 (m, 2H), 1.57-1.28 (m, 16H: including 1.32 (s, 9H)).

[α]_{D}²⁰=−50, c=0.205, chloroform.

### Example 14

### Methyl 6-{[5-(4-methoxyphenyl)-6-pyridin-3-ylfuro[2,3-d]pyrimidin-4-yl]amino}hexanoate

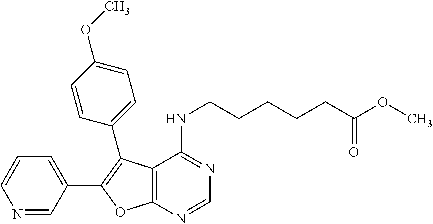

0.23 ml of a 2M aqueous sodium carbonate solution and 34 mg (0.28 mmol) of pyridine-3-boronic acid are added to a mixture of 100 mg (0.24 mmol) of methyl 6-{[6-bromo-5-(4-methoxyphenyl)furo[2,3-d]pyrimidin-4-yl]amino}hexanoate and 8 mg (0.01 mmol) of bis(triphenylphosphine)palladium(II) chloride in 5 ml of DMSO, and the mixture is stirred at 80° C. for 16 hours. After filtration through kieselguhr, the filtrate is isolated on preparative RP-HPLC (gradient: water/acetonitrile). This gives 50 mg (49% of theory) of the desired product.

LC-MS (method 2): Rₜ=2.38 min.; m/z=447 (M+H)⁺¹H-NMR (400 MHz, DMSO-d₆): δ=8.56 (d, 1H), 8.49 (dd, 1H), 8.35 (s, 1H), 7.81 (dd, 1H), 7.46 (d, 2H), 7.41 (dd, 1H), 7.15 (d, 2H), 5.23 (t, 1H), 3.86 (s, 3H), 3.58 (s, 3H), 3.37 (q, 2H), 2.27 (t, 2H), 1.51-1.38 (m, 4H), 1.19-1.13 (m, 2H).

### Example 15

### Methyl 6-{[6-(2-fluoropyridin-3-yl)-5-(4-methoxyphenyl)furo[2,3-d]pyrimidin-4-yl]amino}-hexanoate

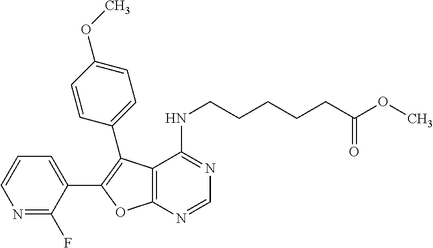

0.34 ml of a 2M aqueous sodium carbonate solution and 118 mg (0.84 mmol) of (2-fluoropyridin-3-yl)boronic acid are added to a mixture of 150 mg (0.24 mmol) of methyl 6-{[6-bromo-5-(4-methoxyphenyl)furo[2,3-d]pyrimidin-4-yl]amino}hexanoate and 19 mg (0.02 mmol) of tetrakis(triphenylphosphine)palladium(0) in 3.5 ml of toluene and 0.8 ml of ethanol, and the mixture is stirred at 70° C. for 16 hours. After filtration through kieselguhr, the filtrate is isolated on preparative RP-HPLC (gradient: water/acetonitrile). This gives 7 mg (4% of theory) of the desired product.

LC-MS (method 3): Rₜ=2.39 min.; m/z=465 (M+H)⁺

### Example 16

### Methyl 6-{[5-(4-methoxyphenyl)-6-(2-thienyl)furo[2,3-d]pyrimidin-4-yl]amino}hexanoate

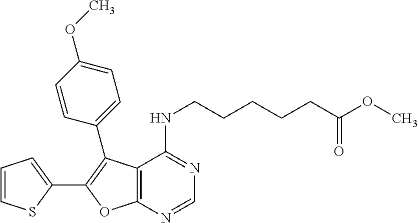

0.50 ml of a 2M aqueous potassium carbonate solution and 80 mg (0.63 mmol) of thiophene-2-boronic acid are added to a mixture of 224 mg (0.50 mmol) of methyl 6-{[6-bromo-5-(4-methoxyphenyl)furo[2,3-d]pyrimidin-4-yl]amino}hexanoate and 18 mg (0.03 mmol) of [1,1′-bis-(diphenylphosphino)-ferrocene]palladium(II) chloride in 2.5 ml of ethylene glycol dimethyl ether, and the mixture is stirred at 90° C. for 16 hours. After filtration through kieselguhr, the filtrate is isolated on preparative RP-HPLC (gradient: water/acetonitrile). This gives 148 mg (66% of theory) of the desired product.

LC-MS (method 7): Rₜ=2.93 min.; m/z=452 (M+H)⁺

¹H-NMR (400 MHz, DMSO-d₆): δ=8.30 (s, 1H), 7.54 (d, 1H), 7.47 (d, 2H), 7.22 (d, 1H), 7.15 (d, 2H), 7.07 (dd, 1H), 5.18 (t, 1H), 3.86 (s, 3H), 3.58 (s, 3H), 3.36 (q, 2H), 2.26 (t, 2H), 1.51-1.37 (m, 4H), 1.19-1.11 (m, 2H).

### General Procedure A: Hydrolysis of Methyl or Ethyl Esters to Give the Corresponding Carboxylic Acid Derivatives

At RT, 1.5 to 10 eq. of sodium hydroxide as a 1 N aqueous solution are added to a solution of a methyl or ethyl ester in THF or THF/methanol (1:1) (concentration about 0.05 to 0.5 mol/l). The mixture is stirred at RT for a period of 0.5-18 h and then neutralized or acidified slightly with 1 N hydrochloric acid. If this results in the precipitation of a solid, the product can be isolated by filtration, washing with water and drying under high vacuum. Alternatively, the target compound is isolated directly from the crude product by preparative RP-HPLC (mobile phase: water/acetonitrile gradient), if appropriate after work-up by extraction with dichloromethane.

The following examples are prepared according to General Procedure A:

| Example | Structure | Analytical data |
|---|---|---|
| 17 | 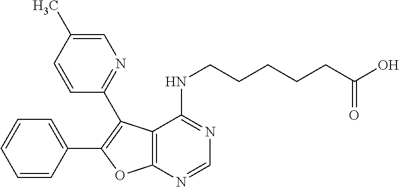 | LC-MS (method 7): Rₜ = 2.49 min.; m/z = 417 (M + H)⁺. |
| 18 | 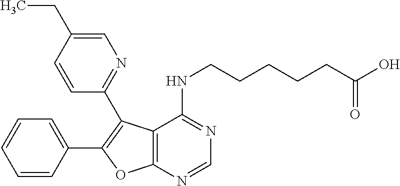 | LC-MS (method 2): Rₜ = 2.64 min.; m/z = 431 (M + H)⁺. ¹H-NMR (400 MHz, CDCl₃): δ = 9.24 (t, 1H), 8.49 (s, 1H), 8.39 (s, 1H), 7.62 (q, 2H), 7.4 (m, 5H), 3.58 (q, 1H), 3.38 (q, 1H), 2.7 (q, 1H), 2.4 (q, 1H), 2.02 (q, 1H), 1.75 (m, 2H), 1.53 (q, 1H), 1.29 (m, 5H), 1.13 (t, 1H). |

### General Procedure B: Cleavage of Tert-Butyl Esters to Give the Corresponding Carboxylic

### Acid Derivatives

At 0° C. to RT, TFA is added dropwise to a solution of the tert-butyl ester in dichloromethane (concentration 0.1 to 1.0 mol/l; additionally optionally a drop of water) until a dichloromethane/TFA ratio of about 2:1 to 1:1 is reached. The mixture is stirred at RT for 1-18 h and then concentrated under reduced pressure. Alternatively, the mixture is diluted with dichloromethane, washed with water and saturated aqueous sodium chloride solution, dried and concentrated under reduced pressure. If required, the reaction product can be purified by preparative RP-HPLC (mobile phase: acetonitrile/water-gradient).

The following examples are prepared according to General Procedure B hergestellt:

| Example | Structure | Analytical data |
|---|---|---|
| 19 | 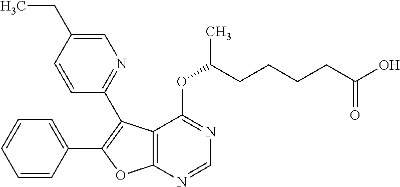 | LC-MS (method 7): Rₜ = 2.59 min; m/z = 446 (M + H)⁺. ¹H-NMR (400 MHz, DMSO- d₆): δ = 8.58 (s, 1H), 8.53 (s, 1H), 7.78 (dd, 1H), 7.57 (dd, 3H), 7.4 (m, 3H), 5.24 (q, 1H), 2.72 (q, 2H), 2.12 (t, 2H), 1.38 (m, 2H), 1.26 (t, 3H), 1.2 (d, 3H), 1.03 (d, 4H). [α]_{D}²⁰ = −45°, c = 0.225, chloroform. |
| 20 | 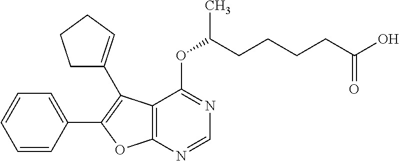 | LC-MS (method 5): Rₜ = 4.16 min; m/z = 407 (M + H)⁺. ¹H-NMR (400 MHz, DMSO- d₆): δ = 11.97 (s, 1H), 8.54 (s, 1H), 7.77 (d, 2H), 7.5 (t, 2H), 7.43 (t, 1H), 6.52 (s, 2H), 5.94 (t, 1H), 5.39 (q, 1H), 2.64 (m, 2H), 2.19 (t, 2H), 2.03 (m, 2H), 1.69 (m, 2H), 1.51 (m, 2H), 1.34 (d, 4H). [α]_{D}²⁰ = −45°, c = 0.16 chloroform |
| 21 | 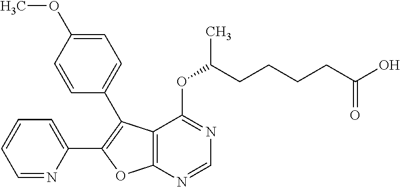 | LC-MS (method 10): Rₜ = 3.46 min; m/z = 448 (M + H)⁺. ¹H-NMR (400 MHz, DMSO- d₆): δ = 12 (s, 1H), 8.6 (s, 1H), 8.57 (d, 1H), 7.83 (t, 1H), 7.57 (d, 1H), 7.42 (d, 2H), 7.36 (q, 1H), 6.97 (d, 2H), 5.3 (m, 1H), 3.8 (m, 3H), 2.15 (t, 2H), 1.51 (q, 2H), 1.41 (m, 2H), 1.24 (d, 3H), 1.17 (m, 2H). [α]_{D}²⁰ = −55°, c = 0.45, chloroform. |

### Example 22

### (6R)-6-[(5-Cyclopent-1-en-1-yl-6-phenylfuro[2,3-d]pyrimidin-4-yl)oxy]heptanoic acid

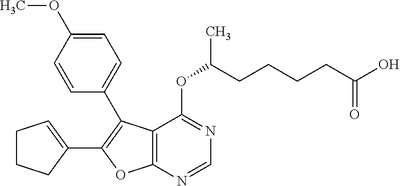

42 mg (0.09 mmol) of tert-butyl (6R)-6-[(5-cyclopent-1-en-1-yl-6-phenylfuro[2,3-d]pyrimidin-4-yl)oxy]heptanoate are dissolved in 130 μl of dichloromethane, 66 μl of trifluoroacetic acid are added and the mixture is stirred at RT for 1 h. The reaction mixture is diluted with dichloromethane and then washed with water and saturated sodium chloride solution. The organic phase is dried over sodium sulfate and concentrated under reduced pressure, and the residue is purified by preparative RP-HPLC (gradient: water/acetonitrile). This gives 3 mg of the title compound (7% of theory).

LC-MS (method 10): Rₜ=4.24 min; m/z=437 (M+H)⁺.

¹H-NMR (400 MHz, DMSO-d₆): δ=11.97 (br. s, 1H), 8.49 (s, 1H), 7.32 (d, 2H), 6.98 (d, 2H), 6.34 (m, 1H), 5.21 (m, 1H), 3.81 (s, 3H), 2.43 (m, 2H), 2.29 (m, 2H), 2.10 (t, 2H), 1.83 (m, 2H), 1.56-1.29 (m, 4H), 1.22-0.98 (m, 5H: including 1.18 (d, 3H)).

### Example 23

### (6R)-6-{[6-Cyclopentyl-5-(4-methoxyphenyl)furo[2,3-d]pyrimidin-4-yl]oxy}heptanoic acid

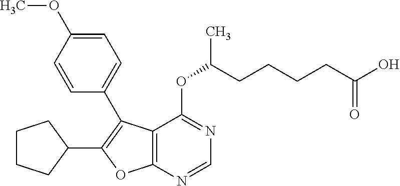

15 mg (0.03 mmol) of tert-butyl (6R)-6-{[6-cyclopentyl-5-(4-methoxyphenyl)furo[2,3-d]pyrimidin-4-yl]oxy}heptanoate are dissolved in 92 μl of dichloromethane, 23 μl of trifluoroacetic acid are added and the mixture is stirred at RT for 30 min. The reaction mixture is diluted with dichloromethane and then washed with water and saturated sodium chloride solution. The organic phase is dried over sodium sulfate, concentrated under reduced pressure and separated by preparative RP-HPLC (gradient: water/acetonitrile). This gives 9 mg (67% of theory) of the title compound.

LC-MS (method 6): Rₜ=2.46 min.; m/z=439 (M+H)⁺.

¹H-NMR (400 MHz, DMSO-d₆): δ=8.43 (s, 1H), 7.35 (d, 2H), 6.95 (d, 2H), 5.34 (m, 1H), 3.88 (s, 3H), 3.25 (m, 1H), 2.72 (t, 2H), 1.95 (m, 4H), 1.89 (m, 2H), 1.71-1.48 (m, 6H), 1.39-1.18 (m, 5H: including 1.29 (d, 3H)).

### Example 24

### (6R)-6-[(5-Cyclohex-1-en-1-yl-6-phenylfuro[2,3-d]pyrimidin-4-yl)oxy]heptanoic acid

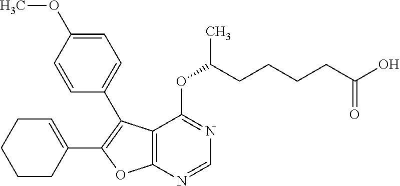

50 mg (0.10 mmol) of tert-butyl (6R)-6-[(5-cyclohex-1-en-1-yl-6-phenylfuro[2,3-d]pyrimidin-4-yl)oxy]heptanoate are dissolved in 1 ml of dichloromethane, and 76 μl of trifluoroacetic acid are added. The reaction mixture is stirred at RT for 1.5 h. The reaction solution is concentrated under reduced pressure and the residue is dissolved in DMSO and purified by preparative RP-HPLC (gradient: water/acetonitrile). This gives 12 mg of the target compound (27% of theory).

LC-MS (method 11): Rₜ=2.71 min; m/z=451 (M+H)⁺.

¹H-NMR (400 MHz, DMSO-d₆): δ=11.98 (br. s, 1H), 8.48 (s, 1H), 7.43 (d, 2H), 6.96 (d, 2H), 6.36 (m, 1H), 5.21 (m, 1H), 3.80 (s, 3H), 2.14 (m, 2H), 2.10 (t, 2H), 2.02 (m, 2H), 1.55 (m, 4H), 1.49-1.32 (m, 4H), 1.22-0.99 (m, 5H: including 1.18 (d, 3H)).

[α]_{D}²⁰=−54°, c=0.510, chloroform.

### Example 25

### (6R)-6-{[5-(4-Ethylcyclohex-1-en-1-yl)-6-phenylfuro[2,3-d]pyrimidin-4-yl]oxy}heptanoic acid

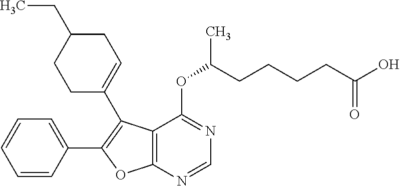

100 mg (0.20 mmol) of tert-butyl (6R)-6-{[5-(4-ethylcyclohex-1-en-1-yl)-6-phenylfuro[2,3-d]pyrimidin-4-yl]oxy}heptanoate are dissolved in 1 ml of dichloromethane, 0.3 ml of trifluoroacetic acid are added and the mixture is stirred at RT for 5 h. The reaction mixture is diluted with dichloromethane and then washed with water and saturated sodium chloride solution. The organic phase is dried over sodium sulfate and concentrated on a rotary evaporator, and the residue is dried under reduced pressure. This gives 76 mg (85% of theory) of the target compound.

LC-MS (method 7): Rₜ=3.39 min; m/z=449 (M+H)⁺.

¹H-NMR (400 MHz, DMSO-d₆): δ=12.00 (br. s, 1H), 8.53 (s, 1H), 7.82 (d, 2H), 7.51 (t, 2H), 7.42 (t, 1H), 5.78 (m, 1H), 5.39 (m, 1H), 2.43-2.13 (m, 5H), 1.96-1.87 (m, 1H), 1.85-1.28 (m, 14H), 0.95 (t, 3H).

[α]_{D}²⁰=−42°, c=0.540, chloroform.

The following compounds are obtained in an analogous manner:

### Example 26

### (6R)-6-{[5-(4-Methylcyclohex-1-en-1-yl)-6-phenylfuro[2,3-d]pyrimidin-4-yl]oxy}heptanoic acid

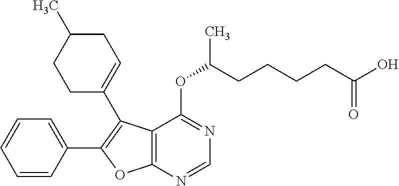

LC-MS (method 7): Rₜ=3.30 min; m/z=435 (M+H)⁺.

¹H-NMR (400 MHz, DMSO-d₆): δ=11.97 (br. s, 1H), 8.53 (s, 1H), 7.82 (d, 2H), 7.51 (t, 2H), 7.41 (t, 1H), 5.78 (m, 1H), 5.39 (m, 1H), 2.44-2.13 (m, 5H), 1.91-1.64 (m, 5H), 1.58-1.27 (m, 8H), 1.05 (d, 3H).

[α]_{D}²⁰=−38°, c=0.130, chloroform.

### Example 27

### (6R)-6-{[5-(4-Methoxycyclohex-1-en-1-yl)-6-phenylfuro[2,3-d]pyrimidin-4-yl]oxy}heptanoic acid

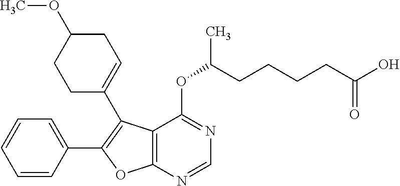

LC-MS (method 6): Rₜ=2.33 min; m/z=451 (M+H)⁺.

¹H-NMR (400 MHz, DMSO-d₆): δ=8.53 (s, 1H), 7.83 (d, 2H), 7.50 (t, 2H), 7.42 (t, 1H), 5.70 (m, 1H), 5.39 (m, 1H), 3.63 (m, 1H), 3.36 (s, 3H), 2.40 (m, 2H), 2.20 (t, 2H), 2.17-1.96 (m, 2H), 1.82 (m, 1H), 1.71 (m, 2H), 1.62-1.30 (m, 8H).

[α]_{D}²⁰=−89°, c=0.075, chloroform.

### Example 28

### (6R)-6-{[5-(4-Ethylcyclohexyl)-6-phenylfuro[2,3-d]pyrimidin-4-yl]oxy}heptanoic acid

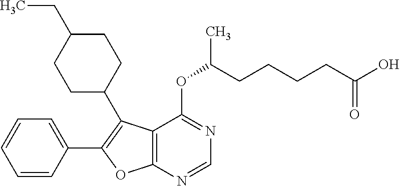

Under an atmosphere of argon, catalytic amounts of 10% palladium on carbon are initially charged in 5 ml of acetic acid, and 70 mg (0.16 mmol) of (6R)-6-{[5-(4-ethylcyclohex-1-en-1-yl)-6-phenylfuro[2,3-d]pyrimidin-4-yl]oxy}heptanoic acid, dissolved in 5 ml of acetic acid, are added. The reaction mixture is stirred under an atmosphere of hydrogen at atmospheric pressure and RT overnight. The catalyst is filtered off through Celite, the filter residue is washed with ethyl acetate and the combined filtrate is extracted twice with water and twice with saturated sodium bicarbonate solution. The organic phase is dried over sodium sulfate and concentrated under reduced pressure. The residue is purified by preparative RP-HPLC (gradient: water/acetonitrile). This gives 13 mg of the title compound (9% of theory).

LC-MS (method 11): Rₜ=3.02 min; m/z=449 (M+H)⁺.

### Example 29

### 6-{[5-(4-Methoxyphenyl)-6-pyridin-3-ylfuro[2,3-d]pyrimidin-4-yl]amino}hexanoic acid

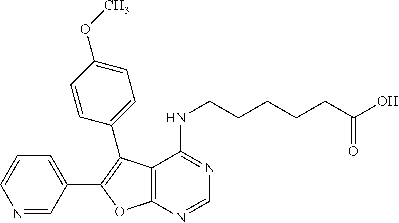

0.2 ml of 1 N aqueous sodium hydroxide solution are added to a solution of 21 mg (0.05 mmol) of methyl 6-{[5-(4-methoxyphenyl)-6-pyridin-3-ylfuro[2,3-d]pyrimidin-4-yl]amino}hexanoate in 0.8 ml of dioxane. The mixture is stirred at room temperature for 16 hours, and 0.2 ml of 1 N aqueous hydrochloric acid and 3 ml of ethyl acetate are added. After removal of the aqueous phase the organic phase is dried over sodium sulfate, filtered and concentrated. This gives 14 mg (68% of theory) of the desired product.

LC-MS (method 3): Rₜ=1.81 min.; m/z=433 (M+H)⁺

¹H-NMR (400 MHz, DMSO-d₆): δ=12.25 (br s, 1H), 8.56 (d, 1H), 8.49 (dd, 1H), 8.35 (s, 1H), 7.81 (dd, 1H), 7.46 (d, 2H), 7.41 (dd, 1H), 7.15 (d, 2H), 5.22 (t, NH), 3.85 (s, 3H), 3.40 (q, 2H), 2.16 (t, 2H), 1.48-1.38 (m, 4H), 1.20-1.12 (m, 2H).

### Example 30

### 6-{[5-(4-Methoxyphenyl)-6-pyridin-2-ylfuro[2,3-d]pyrimidin-4-yl]amino}hexanoic acid

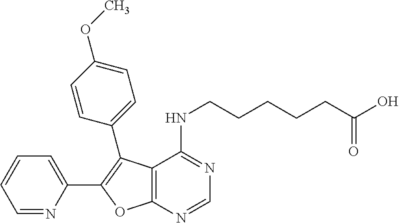

140 mg of potassium hydroxide and 307 mg (2.50 mmol) of pyridin-2-boronic acid (M. D. Sindkhedkar et al. Tetrahedron 2001, 57, 2991-2996) are added to a mixture of 224 mg (0.50 mmol) of methyl 6-{[6-bromo-5-(4-methoxyphenyl)furo[2,3-d]pyrimidin-4-yl]amino}hexanoate and 29 mg (0.03 mmol) of tetrakis(triphenylphosphine)palladium(0) in 3 ml of ethylene glycol dimethyl ether, and the mixture is stirred at 90° C. for 16 hours. After filtration through kieselguhr, the filtrate is isolated on preparative RP-HPLC (gradient: water/acetonitrile). This gives 10 mg (4% of theory) of the desired product.

LC-MS (method 3): Rₜ=1.89 min.; m/z=433 (M+H)⁺

¹H-NMR (400 MHz, CDCl₃): δ=8.41 (d, 1H), 8.27 (s, 1H), 8.45 (dd, 1H), 7.34-7.26 (m, 3H), 7.01 (dd, 1H), 6.92 (d, 2H), 4.82-4.78 (m, NH), 3.78 (s, 3H), 3.23-3.21 (m, 2H), 2.27 (t, 2H), 2.05-1.98 (m, 2H), 1.40-1.07 (m, 4H).

### Example 31

### 6-{[6-(2-Fluoropyridin-3-yl)-5-(4-methoxyphenyl)furo[2,3-d]pyrimidin-4-yl]amino}hexanoic acid

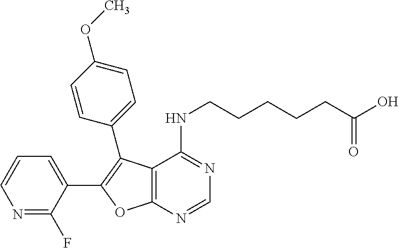

0.05 ml of 1 N aqueous sodium hydroxide solution is added to a solution of 7 mg (0.05 mmol) of methyl 6-{[6-(2-fluoropyridin-3-yl)-5-(4-methoxyphenyl)furo[2,3-d]pyrimidin-4-yl]amino}hexanoate in 0.2 ml of dioxane. The mixture is stirred at room temperature for 16 hours, and 0.05 ml of 1 N aqueous hydrochloric acid, 2 ml of water and 3 ml of dichloromethane are added. After removal of the aqueous phase the organic phase is dried over sodium sulfate, filtered and concentrated. This gives 7 mg (97% of theory) of the desired product.

LC-MS (method 8): Rₜ=2.18 min.; m/z=451 (M+H)⁺

¹H-NMR (400 MHz, DMSO-d₆): δ=12.22 (br s, 1H), 8.37 (s, 1H), 8.29 (dd, 1H), 7.43 (dd, 1H), 7.35-7.30 (m, 2H), 7.24 (dd, 1H), 7.07 (d, 2H), 5.49 (t, NH), 3.81 (s, 3H), 3.41 (q, 2H), 2.21 (t, 2H), 1.49-1.44 (m, 4H), 1.30-1.18 (m, 2H).

### Example 32

### 6-{[5-(4-Methoxyphenyl)-6-(2-thienyl)furo[2,3-d]pyrimidin-4-yl]amino}hexanoic acid

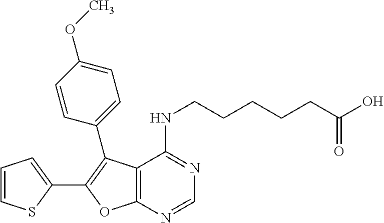

0.33 ml of 1 N aqueous sodium hydroxide solution is added to a solution of 50 mg (0.05 mmol) of methyl 6-{[5-(4-methoxyphenyl)-6-(2-thienyl)furo[2,3-d]pyrimidin-4-yl]amino}hexanoate in 0.6 ml of dioxane. The mixture is stirred at room temperature for 16 hours, and 0.33 ml of 1 N aqueous hydrochloric acid, 2 ml of water and 6 ml of ethyl acetate are added. After removal of the aqueous phase the organic phase is dried over sodium sulfate, washed with diethyl ether, filtered and concentrated. This gives 32 mg (66% of theory) of the desired product.

LC-MS (method 3): R=2.29 min.; m/z=438 (M+H)⁺

¹H-NMR (400 MHz, DMSO-d₆): δ=12.20 (br s, 1H), 8.30 (s, 1H), 7.52 (d, 1H), 7.46 (d, 2H), 7.21 (d, 1H), 7.15 (d, 2H), 7.06 (dd, 1H), 5.18 (t, NH), 3.82 (s, 3H), 3.31 (q, 2H), 2.15 (t, 2H), 1.48-1.40 (m, 4H), 1.28-1.15 (m, 2H).

### Example 33

### (3-{[5-(4-Ethylcyclohex-1-en-1-yl)-6-phenylfuro[2,3-d]pyrimidin-4-yl]amino}phenoxy)acetic acid

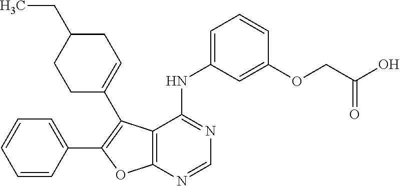

230.8 mg (0.508 mmol) of methyl {3-[(5-bromo-6-phenylfuro[2,3-d]pyrimidin-4-yl)amino]phenoxy}acetate are initially charged in 1.9 ml of DMSO, and under an argon atmosphere 0.5 ml (1.0 mmol) of 2M sodium carbonate solution, 35.7 mg (0.05 mmol) of bis(triphenylphosphine)palladium(II) chloride and 180 mg (0762 mmol) of 2-(4-ethylcyclohex-1-en-1-yl)-4,4,5,5-tetramethyl-1,3,2-dioxaborolane are added in succession. The mixture is heated to 80° C. and stirred vigorously for 2 h. After cooling, the reaction mixture is diluted with a little DMSO and separated directly by preparative RP-HPLC (gradient of acetonitrile and water). This gives 112.8 mg (47.3% of theory) of the title compound.

LC-MS (method 12): Rₜ=2.93 min; m/z=470 (M+H)⁺.

¹H-NMR (400 MHz, DMSO-d₆): δ=8.50 (s, 1H), 7.29 (d, 2H), 7.74 (s, 1H), 7.58-7.51 (m, 1H), 7.48-7.42 (m, 1H), 2.79 (s, 1H), 7.24 (t, 1H), 7.12 (dd, 1H), 6.56 (dd, 1H), 6.29 (s, 1H), 4.11 (s, 2H), 2.42-2.32 (m, 1H), 2.28-2.19 (m, 1H), 2.07-1.89 (m, 2H), 1.63-1.65 (m, 1H), 1.50-1.39 (m, 3H), 0.99 (t, 3H).

The reaction mixture described above also yields the corresponding methyl ester:

### Example 34

### Methyl(3-{[5-(4-ethylcyclohex-1-en-1-yl)-6-phenylfuro[2,3-d]pyrimidin-4-yl]amino}phenoxy)-acetate

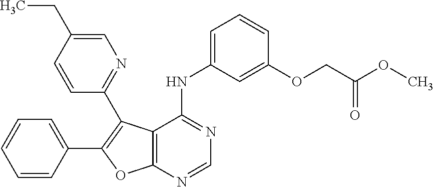

15.1 mg (6.2% of theory) of the title compound are isolated.

LC-MS (method 12): Rₜ=3.17 min; m/z=484 (M+H)⁺.

¹H-NMR (400 MHz, CDCl₃): δ=8.52 (s, 1H) 7.86 (d, 2H), 7.71 (s, 1H), 7.67-7.60 (m, 1H), 7.48-7.35 (m, 3H), 7.28 (t, 1H), 7.07 (d, 1H), 6.67 (dd, 1H), 6.26 (br s, 1H), 4.70 (s, 2H), 3.83 (s, 3H), 2.60-2.51 (m, 1H), 2.43-2.29 (m, 2H), 2.10-1.98 (m, 2H), 1.74-1.56 (m, 2H), 1.54-1.42 (m, including qu with 2H), 1.05 (t, 3H).

### Example 35

### Methyl(3-{[5-(5-ethylpyridin-2-yl)-6-phenylfuro[2,3-d]pyrimidin-4-yl]amino}phenoxy)acetate

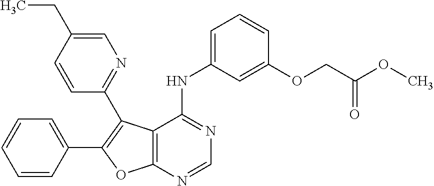

200 mg (0.44 mmol) of methyl {3-[(5-bromo-6-phenylfuro[2,3-d]pyrimidin-4-yl)amino]phenoxy}acetate and 209.3 mg (0.528 mmol) of 5-ethyl-2-(tributylstannyl)pyridine are dissolved in 10 ml of toluene, and 25.4 mg (0.022 mmol) of tetrakis(triphenylphosphine)palladium(0) are added under argon. The mixture is stirred under reflux, after 10 h a further 25 mg (0.02 mmol) of tetrakis(triphenylphosphine)palladium(0) are added and after a further 14 h 209.3 mg (0.528 mmol) of 5-ethyl-2-(tributylstannyl)pyridine and 25 mg (0.02 mmol) of tetrakis(triphenylphosphine)palladium(0) are added. The mixture is stirred under reflux for a further 24 h (i.e. 48 h in total). After cooling, the reaction mixture is filtered through Celite, washed with sat. sodium chloride solution, dried over sodium sulfate and concentrated under reduced pressure. This residue gives, by preparative RP-HPLC (gradient acetonitrile and water), 29.5 mg of the title compound (13.9% of theory).

LC-MS (method 7): Rₜ=3.12 min; m/z=481 (M+H)⁺.

¹H-NMR (400 MHz, 400 MHz, CDCl₃): δ=8.65 (s, 1H), 8.54 (s, 1H), 7.75-7.65 (m, 3H), 7.49-7.40 (m, 5H), 7.38-7.28 (m, 3H), 6.65 (d, 1H), 4.71 (s, 1H), 3.87 (s, 3H), 2.80-2.70 (m, 2H), 1.33 (t, 3H).

### Example 36

### (3-{[5-(5-Ethylpyridin-2-yl)-6-phenylfuro[2,3-d]pyrimidin-4-yl]amino}phenoxy)acetic acid

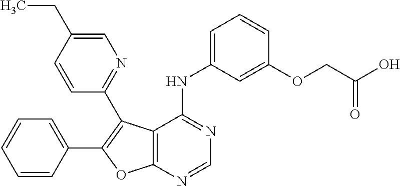

25.5 mg (0.053 mmol) of methyl(3-{[5-(5-ethylpyridin-2-yl)-6-phenylfuro[2,3-d]pyrimidin-4-yl]amino}phenoxy)acetate are dissolved in 0.59 ml of THF, and 0.53 ml of 1 N aqueous sodium hydroxide solution are added at RT. After 40 min at RT, the reaction mixture is acidified slightly (pH 4) by addition of a 1 N hydrochloric acid solution. The mixture is extracted with dichloromethane, and the organic phase is washed with sat. sodium chloride solution, dried and concentrated under reduced pressure. The product is dried under high vacuum. This gives 22.3 mg (90.1% of theory) of the title compound.

LC-MS (method 6): Rₜ=2.32 min; m/z=467 (M+H)⁺.

¹H-NMR (400 MHz, DMSO-d₆): δ=12.32 (s, 1H), 8.86 (s, 1H), 8.54 (s, 1H), 7.71-7.65 (m, 3H), 7.59-7.52 (m, 3H), 7.40-7.15 (m, 5H), 6.65 (d, 1H), 4.74 (s, 2H), 2.72 (qu, 2H), 1.28 (t, 3H).

General Procedure C: Pd-Mediated Coupling of tri-n-butylstannylpyridines with Heteroaryl Bromides

A mixture of heteroaryl bromide and (1.2 to 2.0 eq.) tri-n-butylstannylpyridine in toluene or xylene (about 0.05 to 0.5 mol/l) is repeatedly evacuated and flushed with argon, and about 0.1 eq. of tetrakis(triphenylphosphine)palladium(0) are then added as catalyst. The reaction mixture is from 2 h to 48° C. heated to a temperature from 80° C. to reflux. If required, additional amounts of tri-n-butylstannylpyridine (up to 1.0 eq.) and tetrakis(triphenylphosphine)palladium(0) (about 0.05 eq.) may be added after cooling, and the reaction may be restarted. After cooling, the mixture is concentrated under reduced pressure (alternatively after dilution with dichloromethane and aqueous work-up). The crude product obtained can be purified by chromatography on silica gel (mixtures of cyclohexane/ethyl acetate or dichloromethane/methanol) or by preparative RP-HPLC (mobile phase: water/acetonitrile-gradient); if appropriate, the two purification steps may also be combined.

The following examples are prepared according to General Procedure C:

| Example | Structure | Analytical data |
|---|---|---|
| 37 | 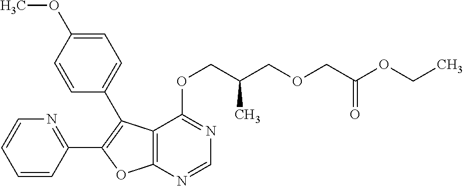 | LC-MS (method 14): Rₜ = 1.32 min; m/z = 478 (M + H)⁺. ¹H-NMR (400 MHz, DMSO-d₆): δ = 8.63 (s, 1H), 8.58 (d, 1H), 7.85 (t, 1H), 7.58 (d, 1H), 7.44 (d, 2H), 7.38 (m, 1H), 6.99 (d, 2H), 4.38 (dd, 1H), 4.32 (dd, 1H), 4.09 (q, 2H), 4.00 (s, 2H), 3.81 (s, 3H), 3.21 (d, 2H), 2.04 (m, 1H), 1.18 (t, 3H), 0.78 (d, 3H). [α]_{D}²⁰ = −8.7°, c = 0.450, chloroform. |
| 38 | 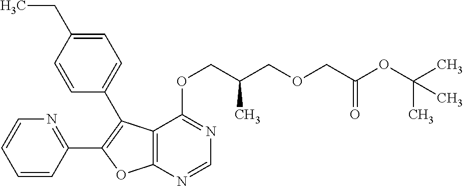 | LC-MS (method 14): Rₜ = 1.61 min; m/z = 504 (M + H)⁺. ¹H-NMR (400 MHz, DMSO-d₆): δ = 8.63 (s, 1H), 8.58 (d, 1H), 7.84 (t, 1H), 7.59 (d, 1H), 7.43 (d, 2H), 7.38 (m, 1H), 7.28 (d, 2H), 4.36-4.28 (m, 2H), 3.82 (s, 2H), 3.17 (d, 2H), 2.68 (q, 2H), 1.99 (m, 1H), 1.39 (s, 9H), 1.23 (t, 3H), 0.72 (d, 3H). [α]_{D}²⁰ = −13.3°, c = 0.50, chloroform. |
| 39 | 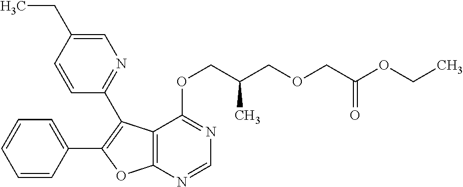 | LC-MS (method 7): Rₜ = 2.74 min; m/z = 476 (M + H)⁺. ¹H-NMR (400 MHz, DMSO-d₆): δ = 8.61 (s, 1H), 8.57 (d, 1H), 7.78 (dd, 1H), 7.60-7.53 (m, 3H), 7.43-7.39 (m, 3H), 4.34 (dd, 1H), 4.27 (dd, 1H), 4.09 (q, 2H), 3.98 (s, 2H), 3.19-3.13 (m, 2H), 2.72 (q, 2H), 1.98 (m, 1H), 1.27 (t, 3H), 1.18 (t, 3H), 0.71 (d, 3H). [α]_{D}²⁰ = −13.7°, c = 0.495, chloroform. |
| 40 | 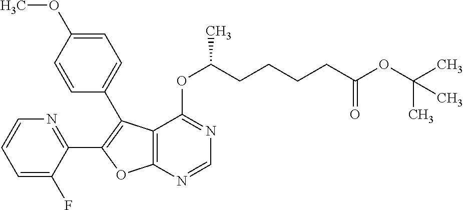 | LC-MS (method 14): Rₜ = 1.64 min; m/z = 522 (M + H)⁺. ¹H-NMR (500 MHz, CDCl₃): δ = 8.58 (s, 1H), 8.48 (s, 1H), 8.43 (d, 1H), 7.48 (t, 1H), 7.31 (d, 2H), 6.90 (d, 2H), 5.41 (m, 1H), 3.88 (s, 3H), 2.14 (t, 2H), 1.70- 1.50 (m, 4H), 1.42 (s, 9H), 1.35-1.20 (m, about 3H), 0.95- 0.84 (m, 2H). [α]_{D}²⁰ = −45.0°, c = 0.655, chloroform. |
| 41 | 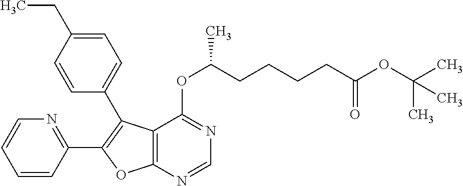 | LC-MS (method 14): Rₜ = 1.76 min; m/z = 502/503 (M + H)⁺. ¹H-NMR (400 MHz, DMSO-d₆): δ = 8.62 (s, 1H), 8.58 (d, 1H), 7.85 (t, 1H), 7.59 (d, 1H), 7.41 (d, 2H), 7.40-7.37 (m, 1H), 7.26 (d, 2H), 5.28 (m, 1H), 2.69 (q, 2H), 2.08 (t, 2H), 1.55-1.35 (m, 4H), 1.35 (s, 9H), 1.20-1.08 (m, 2H). [α]_{D}²⁰ = −63.3°, c = 0.570, chloroform. |
| 42 | 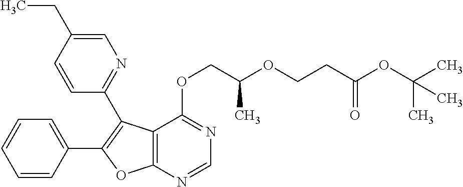 | LC-MS (method 6): Rₜ = 2.56 min; m/z = 504 (M + H)⁺. ¹H-NMR (400 MHz, DMSO-d₆): δ = 8.62 (s, 1H), 8.54 (s, 1H), 7.78 (d, 1H), 7.60-7.53 (m, 3H), 7.43-7.39 (m, 3H), 4.38-4.30 (m, 2H), 3.59 (m, 1H), 3.49- 3.40 (m, 2H), 2.72 (q, 2H), 2.28 (t, 2H), 1.35 (s, 9H), 1.26 (t, 3H), 0.94 (d, 3H). [α]_{D}²⁰ = +26.6°, c = 0.500, chloroform. |
| 43 | 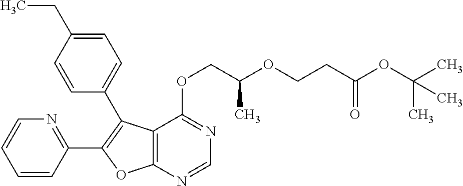 | LC-MS (method 7): Rₜ = 2.56 min; m/z = 504 (M + H)⁺. ¹H-NMR (400 MHz, DMSO-d₆): δ = 8.63 (s, 1H), 8.58 (d, 1H), 7.84 (t, 1H), 7.58 (d, 1H), 7.44 (d, 2H), 7.41-7.38 (m, 1H), 7.26 (d, 2H), 4.39-4.32 (m, 2H), 3.61 (m, 1H), 3.51-3.40 (m, 2H), 2.68 (q, 2H), 2.28 (t, 2H), 1.34 (s, 9H), 1.23 (t, 3H), 0.96 (d, 3H). [α]_{D}²⁰ = +32.5°, c = 0.470, chloroform. |
| 44 | 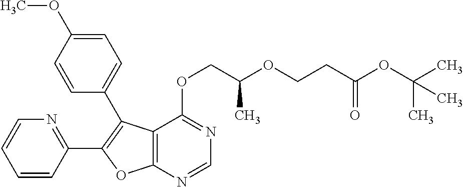 | LC-MS (method 7): Rₜ = 2.77 min; m/z = 506 (M + H)⁺. ¹H-NMR (400 MHz, DMSO-d₆): δ = 8.64 (s, 1H), 8.58 (d, 1H), 7.85 (t, 1H), 7.55 (d, 1H), 7.45 (d, 2H), 7.40-7.35 (m, 1H), 6.98 (d, 2H), 4.39-4.33 (m, 2H), 3.82 (s, 3H), 3.64 (m, 1H), 3.51-3.41 (m, 2H), 2.28 (t, 2H), 1.35 (s, 9H), 1.01 (d, 3H). [α]_{D}²⁰ = +31.6°, c = 0.445, chloroform. |

The following examples are prepared according to General Procedure A:

| Example | Structure | Analytical data |
|---|---|---|
| 45 | 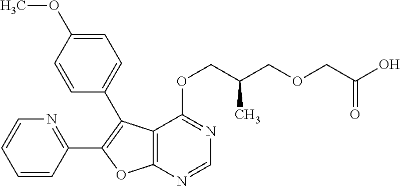 | LC-MS (method 7): Rₜ = 2.15 min; m/z = 450 (M + H)⁺. ¹H-NMR (400 MHz, DMSO-d₆): δ = 12.60 (br s, 1H), 8.62 (s, 1H), 8.58 (d, 1H), 7.85 (t, 1H), 7.57 (d, 1H), 7.45 (d, 2H), 7.38-7.34 (m, 1H), 6.98 (d, 2H), 4.40-4.28 (m, 2H), 3.88 (br s, 2H), 3.82 (s, 3H), 3.22 (d, 2H), 2.02 (m, 1H), 0.77 (d, 3H), [α]_{D}²⁰ = −11.5°, c = 0.450, chloroform. |
| 46 | 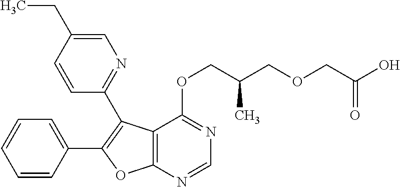 | LC-MS (method 14): Rₜ = 1.18 min; m/z = 448 (M + H)⁺. ¹H-NMR (400 MHz, DMSO-d₆): δ = 12.60 (br s, 1H), 8.61 (s, 1H), 8.55 (s, 1H), 7.89 (dd, 1H), 7.60- 7.54 (m, 3H), 7.44-7.39 (m, 3H), 4.35-4.25 (m, 2H) 3.89 (s, 2H), 3.20-3.10 (m, 2H), 2.71 (q, 2H), 1.98 (m, 1H), 1.27 (t, 3H), 0.69 (d, 3H). [α]_{D}²⁰ = −23.9°, c = 0.535, chloroform. |

General Procedure D: Hydrolysis of tert-butyl Esters to Give the Corresponding Carboxylic Acid Derivatives

At 0° C. to RT, TFA is added dropwise to a solution of the tert-butyl ester in dichloromethane (concentration 0.1 to 1.0 mol/l; additionally optionally a drop of water) until a dichloromethane/TFA ratio of about 2:1 to 1:1 is reached (alternatively, the tert-butyl ester can also be reacted undiluted in TFA). The reaction mixture is stirred at RT to 40° C. for 1-18 h and then concentrated under reduced pressure. Alternatively, the mixture is diluted with dichloromethane, washed with water and sat. sodium chloride solution, dried and concentrated under reduced pressure. If required, the reaction product can be purified by preparative RP-HPLC (mobile phase: acetonitrile/water-gradient).

The following examples are prepared according to General Procedure D:

| Example | Structure | Analytical data |
|---|---|---|
| 47 | 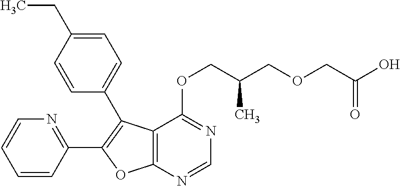 | LC-MS (method 14): Rₜ = 1.23 min; m/z = 448 (M + H)⁺. ¹H-NMR (400 MHz, DMSO-d₆): δ = 12.56 (br s, 1H), 8.63 (s, 1H), 8.59 (d, 1H), 7.84 (t, 1H), 7.59 (d, 1H), 7.42 (d, 2H), 7.39-7.35 (m, 1H), 7.28 (d, 2H), 4.37-4.29 (m, 2H), 3.88 (s, 2H), 3.20-3.14 (m, 2H), 2.68 (q, 2H), 1.99 (m, 1H), 1.22 (t, 3H), 0.73 (d, 3H). [α]_{D}²⁰ = −19.7°, c = 0.530, chloroform. |
| 48 | 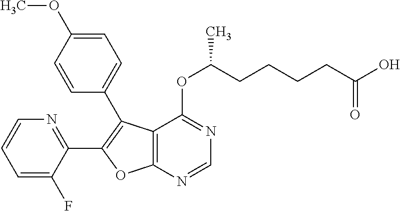 | LC-MS (method 14): Rₜ = 1.24 min; m/z = 466 (M + H)⁺. ¹H-NMR (500 MHz, CDCl₃): δ = 8.58 (s, 1H), 8.48 (s, 1H), 8.41 (d, 1H), 7.48 (t, 1H), 7.31 (d, 2H), 6.91 (d, 2H), 5.41 (m, 1H), 3.87 (s, 3H), 2.28 (t, 2H), 1.70-1.55 (m, about 4H), 1.34 (d, 3H), 1.33-1.25 (m, 2H). [α]_{D}²⁰ = −35.0°, c = 0.225, chloroform. |
| 49 | 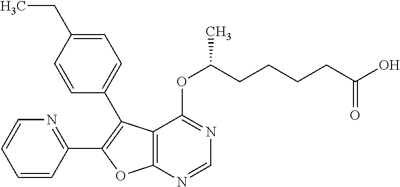 | LC-MS (method 14): Rₜ = 1.34 min; m/z = 446 (M + H)⁺. ¹H-NMR (400 MHz, DMSO-d₆): δ = 11.97 (s, 1H), 8.62 (s, 1H), 8.58 (d, 1H), 7.85 (t, 1H), 7.59 (d, 1H), 7.40 (d, 2H), 7.40-7.37 (m, 1H), 7.26 (d, 2H), 5.29 (m, 1H), 2.68 (q, 2H), 2.12 (t, 2H), 1.55-1.35 (m, 4H), 1.24 (d, 3H), 1.24-1.13 (m, about 2H). [α]_{D}²⁰ = −73.0°, c = 0.460, chloroform. |
| 50 | 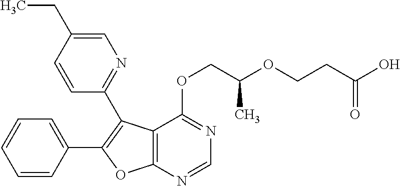 | LC-MS (method 14): Rₜ = 1.15 min; m/z = 448 (M + H)⁺. ¹H-NMR (400 MHz, DMSO-d₆): δ = 12.14 (br s, 1H), 8.61 (s, 1H), 8.53 (s, 1H), 7.78 (dd, 1H), 7.60- 7.54 (m, 3H), 7.44-7.39 (m, 3H), 4.37-4.29 (m, 2H), 3.60 (m, 1H), 3.53-3.42 (m, 2H), 2.72 (q, 2H), 2.30 (t, 2H), 1.27 (t, 3H), 0.95 (d, 3H). [α]_{D}²⁰ = +43.9°, c = 0.505, chloroform. |
| 51 | 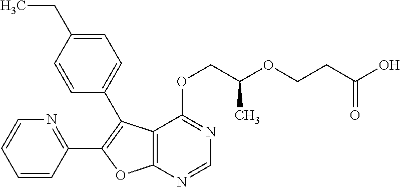 | LC-MS (method 14): Rₜ = 1.21 min; m/z = 448 (M + H)⁺. ¹H-NMR (400 MHz, DMSO-d₆): δ = 12.13 (br s, 1H), 8.64 (s, 1H), 8.58 (d, 1H), 7.84 (t, 1H), 7.59 (d, 1H), 7.43 (d, 2H), 7.40-7.37 (m, 1H), 7.27 (d, 2H), 4.38-4.30 (m, 2H), 3.62 (m, 1H), 3.55-3.41 (m, 2H), 2.69 (q, 2H), 2.31 (t, 2H), 1.24 (t, 3H), 0.97 (d, 3H). [α]_{D}²⁰ = +52.9°, c = 0.520, chloroform. |
| 52 | 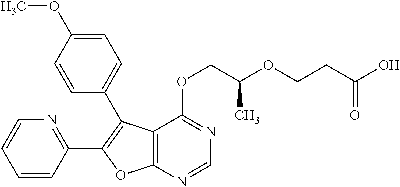 | LC-MS (method 14): Rₜ = 1.06 min; m/z = 450 (M + H)⁺. ¹H-NMR (400 MHz, DMSO-d₆): δ = 12.12 (br s, 1H), 8.63 (s, 1H), 8.58 (d, 1H), 7.85 (t, 1H), 7.57 (d, 1H), 7.44 (d, 2H), 7.40-7.36 (m, 1H), 6.98 (d, 2H), 4.39-4.31 (m, 2H), 3.81 (s, 3H), 3.65 (m, 1H), 3.57-3.45 (m, 2H), 2.30 (t, 2H), 1.00 (d, 3H). [α]_{D}²⁰ = +42.5°, c = 0.485, chloroform. |

### B. ASSESSMENT OF PHARMACOLOGICAL EFFICACY

The pharmacological action of the compounds according to the invention can be demonstrated in the following assays:

### B-1. Studies of Binding to Prostacyclin Receptors (IP Receptors) of Human Thrombocyte Membranes

Thrombocyte membranes are obtained by centrifuging 50 ml of human blood (Buffy coats with CDP Stabilizer, from Maco Pharma, Langen) for 20 min at 160×g. Remove the supernatant (platelet-rich plasma, PRP) and then centrifuge again at 2000×g for 10 min at room temperature. Resuspend the sediment in 50 mM tris(hydroxymethyl)amino-methane, which has been adjusted to a pH of 7.4 with 1 N hydrochloric acid, and store at −20° C. overnight. On the next day, centrifuge the suspension at 80 000×g and 4° C. for 30 min. Discard the supernatant. Resuspend the sediment in 50 mM tris(hydroxy-methyl)aminomethane/hydrochloric acid, 0.25 mM ethylene diamine tetraacetic acid (EDTA), pH 7.4, and then centrifuge once again at 80 000×g and 4° C. for 30 min. Take up the membrane sediment in binding buffer (50 mM tris(hydroxymethyl)-aminomethane/hydrochloric acid, 5 mM magnesium chloride, pH 7.4) and store at −70° C. until the binding test.

For the binding test, incubate 3 nM ³H-Iloprost (592 GBq/mmol, from AmershamBioscience) for 60 min with 300-1000 μg/ml of human thrombocyte membranes per charge (max. 0.2 ml) in the presence of the test substances at room temperature. After stopping, add cold binding buffer to the membranes and wash with 0.1% bovine serum albumin. After adding Ultima Gold Scintillator, quantify the radioactivity bound to the membranes using a scintillation counter. The nonspecific binding is defined as radioactivity in the presence of 1 μM Iloprost (from Cayman Chemical, Ann Arbor) and is as a rule <25% of the bound total radioactivity. The binding data (IC₅₀ values) are determined using the program GraphPad Prism Version 3.02.

Representative results for the compounds according to the invention are shown in Table 1.

TABLE 1

| Example No | IC₅₀ [nM] |
|---|---|
| 3 | 105 |
| 18 | 125 |
| 21 | 53 |
| 24 | 72 |
| 26 | 1871 |
| 32 | 134 |
| 37 | 56 |
| 46 | 63 |
| 48 | 121 |
| 49 | 19 |
| 51 | 193 |

### B-2. IP-Receptor Stimulation on Whole Cells

The IP-agonistic action of test substances is determined by means of the human erythroleukaemia cell line (HEL), which expresses the IP-receptor endogenously [Murray, R., FEBS Letters 1989, 1: 172-174]. For this, the suspension cells (4×10⁷ cells/ml) are incubated with the particular test substance for 5 minutes at 30° C. in buffer [10 mM HEPES (4-(2-hydroxyethyl)-1-piperazineethanesulfonic acid)/PBS (phosphate-buffered saline, from Oxoid, UK)], 1 mM calcium chloride, 1 mM magnesium chloride, 1 mM IBMX (3-isobutyl-1-methylxanthine), pH 7.4. Next, the reaction is stopped by addition of 4° C. cold ethanol and the charges are stored for a further 30 minutes at 4° C. Then the samples are centrifuged at 10 000×g and 4° C. The resultant supernatant is discarded and the sediment is used for determination of the concentration of cyclic adenosine monophosphate (cAMP) in a commercially available cAMP-radioimmunoassay (from IBL, Hamburg). In this test, IP agonists lead to an increase in cAMP concentration, but IP antagonists have no effect. The effective concentration (EC_{S}, values) is determined using the program GraphPad Prism Version 3.02.

### B-3. Inhibition of Thrombocyte Aggregation In Vitro

Inhibition of thrombocyte aggregation is determined using blood from healthy test subjects of both sexes. Mix 9 parts blood with one part 3.8% sodium citrate solution as coagulant. Centrifuge the blood at 900 rev/min for 20 min. Adjust the pH value of the platelet-rich plasma obtained to pH 6.5 with ACD solution (sodium citrate/citric acid/glucose). Then remove the thrombocytes by centrifugation, take up in buffer and centrifuge again. Take up the thrombocyte deposit in buffer and additionally resuspend with 2 mmol/l calcium chloride.

For the measurements of aggregation, incubate aliquots of the thrombocyte suspension with the test substance for 10 min at 37° C. Next, aggregation is induced by adding ADP and is determined by the turbidimetric method according to Born in the Aggregometer at 37° C. [Born G.V.R., J. Physiol. (London) 168, 178-179 (1963)].

B-4. Measurement of blood pressure of anaesthetized rats

Anaesthetize male Wistar rats with a body weight of 300-350 g with thiopental (100 mg/kg i.p.). After tracheotomy, catheterize the arteria femoralis for blood pressure measurement. Administer the test substances as solution, orally by oesophageal tube or intravenously via the femoral vein in a suitable vehicle.

### B-5. PAH Model in the Anaesthetized Dog

In this animal model of pulmonary arterial hypertension (PAH), mongrel dogs having a body weight of about 25 kg are used. Narcosis is induced by slow i.v. administration of 25 mg/kg of sodium thiopental (Trapanal®) and 0.15 mg/kg of alcuronium chloride (Allo-ferin®) and maintained during the experiment by continuous infusion of 0.04 mg/kg/h of Fentanyl®, 0.25 mg/kg/h of droperidol (Dehydrobenzperidol®) and 15 μg/kg/h of alcuronium chloride (Alloferin®). Reflectory effects on the pulse by lowering of the blood pressure are kept to a minimum by autonomous blockage [continuous infusion of atropin (about 10 μg/kg/h) and propranolol (about 20 μg/kg/h)]. After intubation, the animals are ventilated using a ventilator with constant tidal volume such that an end-tidal CO₂ concentration of about 5% is reached. Ventilation takes place with ambient air enriched with about 30% oxygen (normoxa). For measuring the hemodynamic parameters, a liquid-filled catheter is implanted into the femoralis artery for measuring the blood pressure. A double-lumiger Swan-Ganz® catheter is introduced via the jugulara vein into the pulmonary artery (distal lumen for measuring the pulmonary arterial pressure, proximal lumen for measuring the central venus pressure). The left-ventricular pressure is measured following introduction of a micro-tip catheter (Millar® Instruments) via the carotis artery into the left ventricle, and from this, the dP/dt value is derived as a measure for the contractility. Substances are administered i.v. via the femoralis vein. The hemodynamic signals are recorded and evaluated using pressure sensors/amplifiers and PONEMAH® as data acquisition software.

To induce acute pulmonary hypertension, the stimulus used is either hypoxia or continuous infusion of thromboxan A₂ or a thromboxan A₂ analog. Acute hypoxia is induced by gradually reducing the oxygen in the ventilation air to about 14%, such that the mPAP increases to values of >25 mm Hg. If the stimulus used is a thromboxan A_{2 analog,} 0.21-0.32 μg/kg/min of U-46619 [9,11-dideoxy-9α, 11α-epoxy-methanoprostaglandin F_{2α}, (from Sigma)] is infused to increase the mPAP to >25 mm Hg.

B-6. PAH Model in Anaesthetized Göttingen Minipig

In this animal model of pulmonary arterial hypertension (PAH), Göttingen minipigs having a body weight of about 25 kg are used. Narcosis is induced by 30 mg/kg of ketamine (Ketavet®) i.m., followed by i.v. administration of 10 mg/kg of sodium thiopental (Trapanal®); during the experiment, it is maintained by inhalation narcosis using enfluran (2-2.5%) in a mixture of ambient air enriched with about 30-35% oxygen/N₂O (1:1.5). For measuring the hemodynamic parameters, a liquid-filled catheter is implanted into the carotis artery for measuring the blood pressure. A double-lumiger Swan-Ganz® catheter is introduced via the jugulara vein into the pulmonary artery (distal lumen for measuring the pulmonary arterial pressure, proximal lumen for measuring the central venus pressure). The left-ventricular pressure is measured following introduction of a micro-tip catheter (Millar® Instruments) via the carotis artery into the left ventricle, and from this, the dP/dt value is derived as a measure for the contractility. Substances are administered i.v. via the femoralis vein. The hemodynamic signals are recorded and evaluated using pressure sensors/amplifiers and PONEMAH® as data acquisition software.

To induce acute pulmonary hypertension, the stimulus used is continuous infusion of a thromboxan A₂ analog. Here, 0.12-0.14 μg/kg/min of U-46619 [9,11-dideoxy-9α, 11α-epoxymethanoprostaglandin F_{2α} (from Sigma)] is infused to increase the mPAP to >25 mm Hg.

### C. EXEMPLARY EMBODIMENTS OF PHARMACEUTICAL COMPOSITIONS

The compounds of the invention can be converted into pharmaceutical preparations in the following ways:

### Tablet:

### Composition:

100 mg of the compound of the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

### Production:

The mixture of compound of the invention, lactose and starch is granulated with a 5% strength solution (m/m) of the PVP in water. The granules are mixed with the magnesium stearate for 5 minutes after drying. This mixture is compressed with a conventional tablet press (see above for format of the tablet). A guideline compressive force for the compression is 15 kN.

Suspension which can be Administered Orally:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

### Composition:

500 mg of the compound of the invention, 2.5 g of polysorbate and 97 g of polyethylene glycol 400.20 g of oral solution correspond to a single dose of 100 mg of the compound according to the invention.

### Production:

The compound of the invention is suspended in the mixture of polyethylene glycol and polysorbate with stirring. The stirring process is continued until the compound according to the invention has completely dissolved.

i.v. Solution:

The compound of the invention is dissolved in a concentration below the saturation solubility in a physiologically tolerated solvent (e.g. isotonic saline solution, 5% glucose solution and/or 30% PEG 400 solution). The solution is sterilized by filtration and used to fill sterile and pyrogen-free injection containers.

1. A compound of the formula (I) 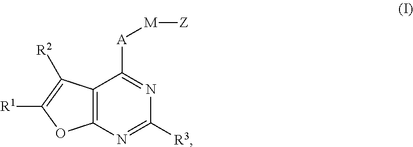
in which
A represents O or N—R⁴,
where
R⁴ represents hydrogen, (C₁-C₆)-alkyl, (C₃-C₇)-cycloalkyl or (C₄-C₇)-cycloalkenyl,
M represents a group of the formula 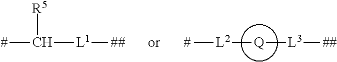
where
# represents the point of attachment to A,
## represents the point of attachment to Z,
R⁵ represents hydrogen or (C₁-C₄)-alkyl,
where alkyl may be substituted by hydroxyl or amino,
L¹ represents (C₁-C₇)-alkanediyl, (C₂-C₇)-alkenediyl or a group of the formula *-L^{1A}-V-L^{1B}-**,
where alkanediyl and alkenediyl may be mono- or disubstituted by fluorine,
where
* represents the point of attachment to —CHR⁵,
** represents the point of attachment to Z,
L^{1A} represents (C₁-C₅)-alkanediyl,
where alkanediyl may be substituted by 1 or 2 substituents independently of one another selected from the group consisting of (C₁-C₄)-alkyl and (C₁-C₄)-alkoxy,
L^{1B} represents a bond or (C₁-C₃)-alkanediyl,
where alkanediyl may be mono- or disubstituted by fluorine,
and
V represents O or N—R⁶,
where
R⁶ represents hydrogen, (C₁-C₆)-alkyl or (C₃-C₇)-cycloalkyl,
L² represents a bond or (C₁-C₄)-alkanediyl,
Q represents (C₃-C₇)-cycloalkyl, (C₄-C₇)-cycloalkenyl, 5- to 7-membered heterocyclyl, phenyl or 5- or 6-membered heteroaryl,
where cycloalkyl, cycloalkenyl, heterocyclyl, phenyl and heteroaryl may each be substituted by 1 or 2 substituents independently of one another selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino,
where alkyl may be substituted by a substituent selected from the group consisting of hydroxyl, (C₁-C₄)-alkoxy, amino, mono-(C₁-C₄)-alkylamino and di-(C₁-C₄)-alkylamino, and
L³ represents (C₁-C₄)-alkanediyl or (C₂-C₄)-alkenediyl,
where alkanediyl may be mono- or disubstituted by fluorine, and
where a methylene group of the alkanediyl group may be replaced by O or N—R⁷,
where
R⁷ represents hydrogen, (C₁-C₆)-alkyl or (C₃-C₇)-cycloalkyl,
Z represents a group of the formula 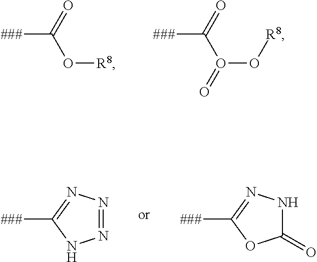
where
### represents the point of attachment to the group L¹ or L³,
and
R⁸ represents hydrogen or (C₁-C₄)-alkyl,
and either
R¹ represents (C₃-C₇)-cycloalkyl, (C₄-C₇)-cycloalkenyl, 5- to 7-membered heterocyclyl or 5- or 6-membered heteroaryl,
where cycloalkyl, cycloalkenyl, heterocyclyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
and
R² represents (C₃-C₇)-cycloalkyl, (C₄-C₇)-cycloalkenyl, 5- to 7-membered heterocyclyl, phenyl or 5- or 6-membered heteroaryl,
where cycloalkyl, cycloalkenyl, heterocyclyl, phenyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
or
two substituents attached to adjacent carbon atoms of a phenyl ring together form a group of the formula —O—CH₂—O—, —O—CHF—O—, —O—CF₂—O—, —O—CH₂—CH₂—O— or —O—CF₂—CF₂—O—,
or
R¹ represents (C₃-C₇)-cycloalkyl, (C₄-C₇)-cycloalkenyl, 5- to 7-membered heterocyclyl, phenyl or 5- or 6-membered heteroaryl,
where cycloalkyl, cycloalkenyl, heterocyclyl, phenyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
or
two substituents attached to adjacent carbon atoms of a phenyl ring together form a group of the formula —O—CH₂—O—, —O—CHF—O—, —O—CF₂—O—, —O—CH₂—CH₂—O— or —O—CF₂—CF₂—O—,
and
R² represents (C₃-C₇)-cycloalkyl, (C₄-C₇)-cycloalkenyl, 5- to 7-membered heterocyclyl or 5- or 6-membered heteroaryl,
where cycloalkyl, cycloalkenyl, heterocyclyl and heteroaryl may each be substituted by 1 to 3 substituents independently of one another selected from the group consisting of halogen, cyano, nitro, formyl, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
where alkyl and alkoxy may each be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
and
R³ represents hydrogen, (C₁-C₄)-alkyl or cyclopropyl, or one of its salts, solvates or solvates of the salts.

2. The compound of the formula (I) as claimed in claim 1 in which
A represents O or N—R⁴,
where
R⁴ represents hydrogen, (C₁-C₄)-alkyl or cyclopropyl,
M represents a group of the formula 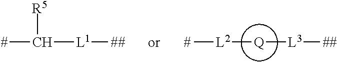
where
# represents the point of attachment to A,
## represents the point of attachment to Z,
R⁵ represents hydrogen or (C₁-C₃)-alkyl,
where alkyl may be substituted by hydroxyl or amino,
L¹ represents (C₃-C₇)-alkanediyl, (C₃-C₇)-alkenediyl or a group of the formula *-L^{1A}-V-L^{1B}-**,
where alkanediyl and alkenediyl may be mono- or disubstituted by fluorine,
where
* represents the point of attachment to —CHR⁵,
** represents the point of attachment to Z,
L^{1A} represents (C₁-C₃)-alkanediyl,
where alkanediyl may be substituted by 1 or 2 substituents independently of one another selected from the group consisting of methyl and ethyl,
L^{1B} represents (C₁-C₃)-alkanediyl,
where alkanediyl may be mono- or disubstituted by fluorine,
and
V represents O or N—R⁶,
where
R⁶ represents hydrogen, (C₁-C₃)-alkyl or cyclopropyl,
L² represents a bond or (C₁-C₃)-alkanediyl,
Q represents (C₃-C₆)-cycloalkyl, (C₄-C₆)-cycloalkenyl, 5- or 6-membered heterocyclyl or phenyl,
where cycloalkyl, cycloalkenyl, heterocyclyl and phenyl may each be substituted by 1 or 2 substituents independently of one another selected from the group consisting of fluorine, chlorine, (C₁-C₃)-alkyl, trifluoromethyl, hydroxyl, methoxy, ethoxy, trifluoromethoxy, amino, methylamino, dimethylamino, ethylamino and diethylamino,
and
L³ represents (C₁-C₃)-alkanediyl, (C₂-C₃)-alkenediyl or a group of the formula -W—CR⁹R¹⁰, -W—CH₂—CR⁹R¹⁰ or -CH₂—W—CR⁹R¹⁰,
where alkanediyl may be mono- or disubstituted by fluorine,
where
 represents the point of attachment to ring Q,
 represents the point of attachment to the group Z,
W represents O or N—R¹¹,
where
R¹¹ represents hydrogen, (C₁-C₃)-alkyl or cyclopropyl,
R⁹ represents hydrogen or fluorine,
and
R¹ represents hydrogen or fluorine,
Z represents a group of the formula 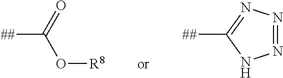
where
### represents the point of attachment to the group L¹ or L³,
and
R⁸ represents hydrogen, methyl or ethyl,
and either
R¹ represents (C₄-C₆)-cycloalkyl, (C₄-C₆)-cycloalkenyl, 5- or 6-membered heterocyclyl or 5- or 6-membered heteroaryl,
where cycloalkyl, cycloalkenyl, heterocyclyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
and
R² represents phenyl or 5- or 6-membered heteroaryl,
where phenyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
or
two substituents attached to adjacent carbon atoms of a phenyl ring together form a group of the formula —O—CH₂—O—, —O—CHF—O— or —O—CF₂—O—,
or
R¹ represents phenyl or 5- or 6-membered heteroaryl,
where phenyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
or
two substituents attached to adjacent carbon atoms of a phenyl ring together form a group of the formula —O—CH₂—O—, —O—CHF—O— or —O—CF₂—O—,
and
R² represents (C₄-C₆)-cycloalkyl, (C₄-C₆)-cycloalkenyl, 5- or 6-membered heterocyclyl or 5- or 6-membered heteroaryl,
where cycloalkyl, cycloalkenyl, heterocyclyl and heteroaryl may each be substituted by 1 to 3 substituents independently of one another selected from the group consisting of halogen, cyano, nitro, formyl, (C₁-C₄)-alkyl, (C₂-C₄)-alkenyl, (C₂-C₄)-alkynyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₄)-alkylcarbonyl, amino, mono-(C₁-C₄)-alkylamino, di-(C₁-C₄)-alkylamino and (C₁-C₄)-alkylcarbonylamino,
and
R³ represents hydrogen or (C₁-C₃)-alkyl,
or one of its salts, solvates or solvates of the salts.

3. The compound of the formula (1) as claimed in claim 1 in which
A represents O or NH,
M represents a group of the formula 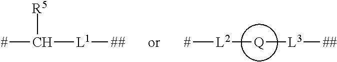
where
# represents the point of attachment to A,
## represents the point of attachment to Z,
R⁵ represents hydrogen, methyl or ethyl,
L¹ represents (C₃-C₇)-alkanediyl, (C₃-C₇)-alkenediyl or a group of the formula*-L^{1A}-V-L^{1B}-**,
where
* represents the point of attachment to —CHR⁵,
** represents the point of attachment to Z,
L^{1A} represents (C₁-C₃)-alkanediyl,
where alkanediyl may be substituted by 1 or 2 substituents independently of one another selected from the group consisting of methyl and ethyl,
L^{1B} represents (C₁-C₃)-alkanediyl,
and
V represents O or N—CH₃,
L² represents a bond, methylene, ethane-1,1-diyl or ethane-1,2-diyl,
Q represents cyclobutyl, cyclopentyl, cyclohexyl, piperidinyl or phenyl,
where cyclobutyl, cyclopentyl, cyclohexyl, piperidinyl and phenyl may each be substituted by 1 or 2 substituents independently of one another selected from the group consisting of fluorine, methyl, ethyl, trifluoromethyl, hydroxyl, methoxy and ethoxy,
and
L³ represents (C₁-C₃)-alkanediyl or a group of the formula -W—CH₂ or -W—CH₂CH₂-,
where
 represents the point of attachment to ring Q,
 represents the point of attachment to the group Z,
W represents O or N—R¹¹,
where
R¹¹ represents hydrogen or (C₁-C₃)-alkyl,
Z represents a group of the formula 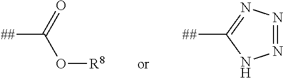
where
### represents the point of attachment to the group L¹ or L³,
and either
R¹ represents (C₄-C₆)-cycloalkyl, (C₄-C₆)-cycloalkenyl or 5- or 6-membered heteroaryl,
where cycloalkyl, cycloalkenyl and heteroaryl groups may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
and
R² represents phenyl,
where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
or
R¹ represents phenyl,
where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
and
R² represents (C₄-C₆)-cycloalkyl, (C₄-C₆)-cycloalkenyl or 5- or 6-membered heteroaryl,
where cycloalkyl, cycloalkenyl and heteroaryl may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
and
R³ represents hydrogen or methyl,
or one of its salts, solvates or solvates of the salts.

4. The compound of the formula (I) as claimed in claim 1 in which
A represents O or NH,
M represents a group of the formula 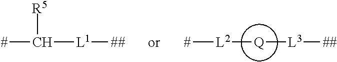
where
# represents the point of attachment to A,
## represents the point of attachment to Z,
R⁵ represents hydrogen, methyl or ethyl,
L¹ represents (C₃-C₇)-alkanediyl, (C₃-C₇)-alkenediyl or a group of the formula *-L^{1A}-V-L^{1B}-**,
where
* represents the point of attachment to —CHR⁵,
** represents the point of attachment to Z,
L^{1A} represents (C₁-C₃)-alkanediyl,
L^{1B} represents (C₁-C₃)-alkanediyl,
and
V represents O or N—CH₃,
L² represents a bond, methylene, ethane-1,1-diyl or ethane-1,2-diyl,
Q represents cyclobutyl, cyclopentyl, cyclohexyl, cyclopentenyl, pyrrolodinyl, piperidinyl, tetrahydrofuranyl, tetrahydropyranyl, morpholinyl or phenyl,
where cyclobutyl, cyclopentyl, cyclohexyl, cyclopentenyl, pyrrolodinyl, piperidinyl, tetrahydrofuranyl, tetrahydropyranyl, morpholinyl and phenyl may each be substituted by 1 or 2 substituents independently of one another selected from the group consisting of fluorine, methyl, ethyl, trifluoromethyl, hydroxyl, methoxy and ethoxy
and
L³ represents (C₁-C₃)-alkanediyl or a group of the formula -W—CH₂ or W-—CH₂CH₂-,
where
 represents the point of attachment to ring Q,
 represents the point of attachment to the group Z,
W represents O or N—R¹¹,
where
R¹¹ represents hydrogen or (C₁-C₃)-alkyl,
Z represents a group of the formula 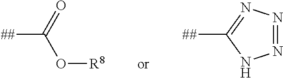
where
### represents the point of attachment to the group L¹ or L³,
and either
R¹ represents (C₄-C₆)-cycloalkyl, (C₄-C₆)-cycloalkenyl or 5- or 6-membered heteroaryl,
where cycloalkyl, cycloalkenyl and heteroaryl may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
and
R² represents phenyl,
where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
or
R¹ represents phenyl,
where phenyl may be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
and
R² represents (C₄-C₆)-cycloalkyl, (C₄-C₆)-cycloalkenyl or 5- or 6-membered heteroaryl,
where cycloalkyl, cycloalkenyl and heteroaryl may each be substituted by 1 or 2 substituents independently of one another selected from the group consisting of fluorine, chlorine, cyano, formyl, (C₁-C₄)-alkyl, (C₂-C₄)-alkenyl, (C₁-C₄)-alkoxy, trifluoromethyl and trifluoromethoxy,
and
R³ represents hydrogen or methyl,
or one of its salts, solvates or solvates of the salts.

5. The compound of the formula (I) as claimed in claim 1 in which
A represents O or NH,
M represents a group of the formula 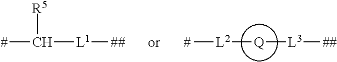
where
# represents the point of attachment to A,
## represents the point of attachment to Z,
R⁵ represents hydrogen or methyl,
L¹ represents butane-1,4-diyl or pentane-1,5-diyl,
L² represents a bond or methylene,
Q represents cyclopentyl, cyclohexyl or phenyl,
and
L³ represents methylene, ethane-1,2-diyl, propane-1,3-diyl or a group of the formula -O—CH₂- or -O—CH₂—CH₂-,
where
 represents the point of attachment to ring Q,
 represents the point of attachment to the group Z,
Z represents a group of the formula 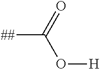
where
### represents the point of attachment to the group L¹ or L³,
and either
R¹ represents cyclopent-1-en-1-yl, cyclohexen-1-en-1-yl, thienyl or pyridyl,
where cyclopent-1-en-1-yl and cyclohexen-1-en-1-yl may each be monosubstituted by fluorine,
and
where thienyl and pyridyl may each be substituted by a substituent selected from the group consisting of fluorine, chlorine, methyl and trifluoromethyl,
and
R² represents phenyl,
where phenyl may be substituted by a substituent selected from the group consisting of methyl, ethyl, vinyl, trifluoromethyl, methoxy, ethoxy and trifluoromethoxy,
or
R¹ represents phenyl,
where phenyl may be substituted by a substituent selected from the group consisting of fluorine, chlorine, methyl, trifluoromethyl, methoxy and trifluoromethoxy,
and
R² represents cyclopent-1-en-1-yl, cyclohexen-1-en-1-yl or pyridyl,
where cyclopent-1-en-1-yl and cyclohexen-1-en-1-yl may each be substituted by a substituent selected from the group consisting of methyl, ethyl, trifluoromethyl and methoxy,
and
where pyridyl may be substituted by a substituent selected from the group consisting of methyl, ethyl, vinyl, trifluoromethyl, methoxy, ethoxy and trifluoromethoxy,
and
R³ represents hydrogen,
or one of its salts, solvates or solvates of the salts.

6. A process for preparing a compound of claim 1 in which Z represents —COOH, characterized in that either
[A] a compound of the formula (II-A) 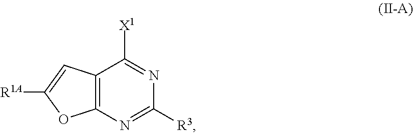
in which R³ has the meaning given in claim 1
and
R^{1a} represents (C₄-C₇)-cycloalkenyl, 5- or 6-membered heteroaryl or phenyl,
where the cycloalkenyl, heteroaryl and phenyl groups mentioned may be substituted within the scope of the meanings mentioned in claim 1,
and
X¹ represents a leaving group,
is reacted in the presence of a base, with a compound of the formula (III) 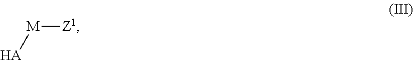
in which A and M each have the meanings given in claim 1
and
Z¹ represents cyano or a group of the formula COOR^{8A},
where
R^{8A} represents (C₁-C₄)-alkyl,
to give a compound of the formula (IV-A) 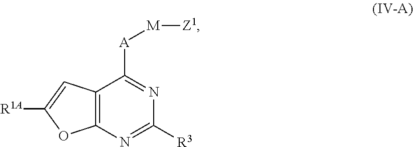
in which A, M, Z¹, R^{1A} and R³ each have the meanings given above,
which is then brominated in an inert solvent to give a compound of the formula (V-A) 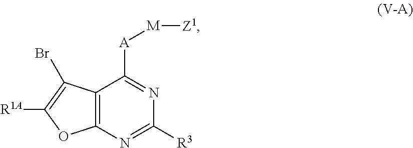
in which A, M, Z¹, R^{1A} and R³ have the meanings given above, which is coupled in an inert solvent in the presence of a base and a suitable palladium catalyst with a compound of the formula (VI-A) 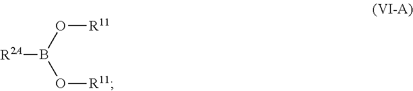
in which
R^{2A} represents (C₄-C₇)-cycloalkenyl or 5- or 6-membered heteroaryl,
where the cycloalkenyl and heteroaryl groups mentioned may be substituted within the scope of the meanings mentioned in claim 1,
and
R¹¹ represents hydrogen or the two radicals R¹¹ together form a —C(CH₃)₂—C(CH₃)₂— bridge,
to give a compound of the formula (VII-A) 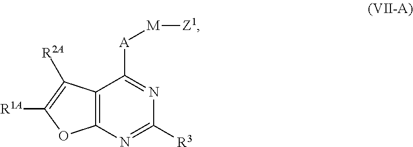
in which A, M, Z¹, R^{1A}, R^{2A} and R³ have the meanings given above,
or
[B] a compound of the formula (II-B) 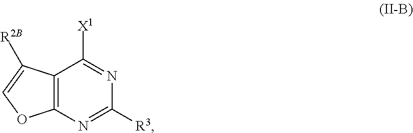
in which R³ has the meaning given in claim 1
and
X¹ represents a leaving group,
and
R^{2B} represents (C₄-C₇)-cycloalkenyl, 5- or 6-membered heteroaryl or phenyl,
where the cycloalkenyl, heteroaryl and phenyl groups mentioned may be substituted within the scope of the meanings mentioned in claim 1,
is reacted in the presence of a base with a compound of the formula (III) to give a compound of the formula (IV-B) 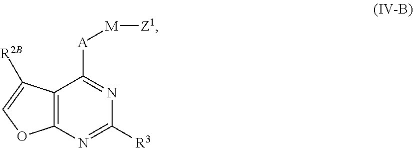
in which R^{2B} and R³ each have the meanings given above
and A and M each have the meanings given in claim 1
and
Z¹ represents cyano or a group of the formula COOR^{8A},
where
R^{8A} represents (C₁-C₄)-alkyl,
which is then brominated in an inert solvent to give a compound of the formula (V-B) 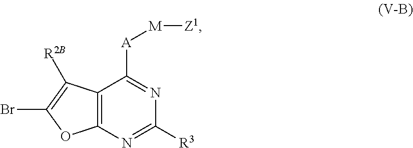
in which A, M, Z¹, R^{2B} and R³ have the meanings given above,
which is coupled in an inert solvent in the presence of a base and a suitable palladium catalyst with a compound of the formula (VI-B) 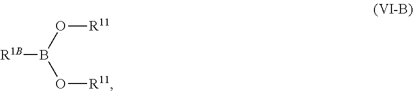
in which
R^{1B} represents (C₄-C₇)-cycloalkenyl or 5- or 6-membered heteroaryl,
where the cycloalkenyl and heteroaryl groups mentioned may be substituted within the scope of the meanings mentioned in claim 1,
and
R¹¹ represents hydrogen or the two radicals R¹¹ together form a —C(CH₃)₂—C(CH₃)₂-bridge.
to give a compound of the formula (VII-B) 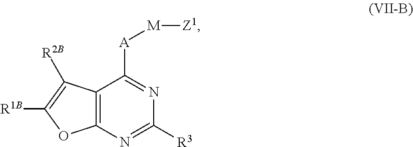
in which A, M, Z¹, R^{1B}, R^{2B} and R³ have the meanings indicated above,
or
[C] a compound of the formula (V-A) is coupled in an inert solvent in the presence of a suitable palladium catalyst with a compound of the formula (VI-C) 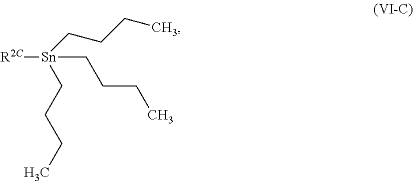
in which
R^{2c} represents 5- or 6-membered heteroaryl,
where heteroaryl may be substituted within the scope of the meaning mentioned in claim 1,
to give a compound of the formula (VII-C) 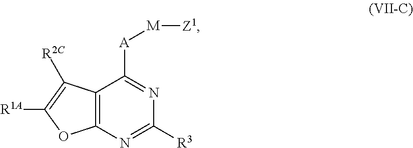
in which R^{2C} has the meaning indicated above,
and A, M, and R³ each have the meanings given in claim 1, R^{1A} represents (C₄-C₇)-cycloalkenyl, 5- or 6-membered heteroaryl or phenyl,
where the cycloalkenyl, heteroaryl and phenyl groups mentioned may be substituted within the scope of the meanings mentioned in claim 1,
and
Z¹ represents cyano or a group of the formula COOR^{8A},
where
R^{8A} presents (C₁-C₄)-alkyl,
or
[D] a compound of the formula (V-B) is coupled in an inert solvent in the presence of a suitable palladium catalyst with a compound of the formula (VI-D)
in which 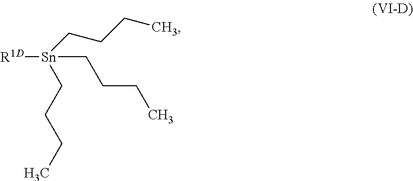
R^{1p} represents 5- or 6-membered heteroaryl,
where heteroaryl may be substituted within the scope of the meaning mentioned in claim 1,
to give a compound of the formula (VII-D) 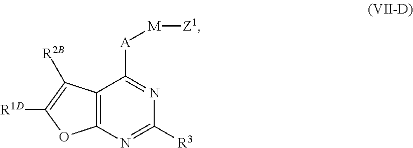
in which R^{1D} has the meaning indicated above,
and A, M, and R³ each have the meanings given in claim 1,
R^{1A} represents C₄-C₇)-cycloalkenyl 5- or 6-membered heteroaryl or phenyl,
where the cycloalkenyl, heteroaryl and phenyl groups mentioned may be substituted within the scope of the meanings mentioned in claim 1,
R^{2B} represents C₄-C₇-cycloalkenyl 5- or 6-membered heteroaryl or phenyl,
where the cycloalkenyl, heteroaryl and phenyl groups mentioned may be substituted within the scope of the meanings mentioned in claim 1,
and
Z¹ represents cyano or a group of the formula COOR^{8A},
where
R^{8A} represents C₁-C₄-alkyl
or
[E] a compound of the formula (V-A) is coupled in an inert solvent with 4,4,4′,4′,5,5,5′,5′-octamethyl-2,2′-bi-1,3,2-dioxoborolane (bis(pinacolato)-diboron) to give a compound of the formula (VI-E) 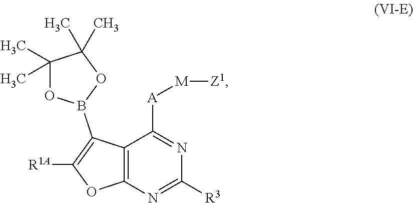
in which A, M, and R³ each have the meanings given in claim 1,
R^{1A} represents C₄-C₇-cycloalkenyl 5- or 6-membered heteroaryl or phenyl,
where the cycloalkenyl, heteroaryl and phenyl groups mentioned may be
substituted within the scope of the meanings mentioned in claim 1,
and
Z¹ represents cyano or a group of the formula COOR^{8A},
where
R^{8A} represents C₁-C₄-alkyl
which is then coupled with a compound of the formula (VII-E)
R^{2C}—X² (VII-E)
in which
R^{2c} represents 5- or 6-membered heteroaryl,
where heteroaryl may be substituted within the scope of the meaning mentioned in claim 1,
and
X² represents a leaving group,
in an inert solvent, to give a compound of the formula (VII-C),
and the compound of the formulae (VII-A), (VII-B), (VII-C) or (VII-D) is then converted by hydrolysis of the cyano or ester group Z¹ into a carboxylic acid of the formula (I-1) 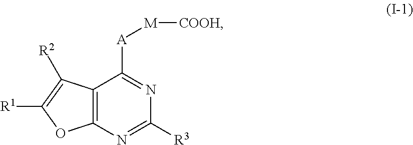
in which A, M, R¹, R² and R³ have the meanings given above,
which is optionally reacted with the appropriate (i) solvents and/or (ii) bases or acids to give its solvate, salt and/or solvate of a salt.

7. (canceled)

8. (canceled)

9. (canceled)

10. A pharmaceutical composition comprising a compound of claim 1 in combination with an inert non-toxic pharmaceutically acceptable auxiliary.

11. A pharmaceutical composition comprising a compound of claim 1 in combination with a further active compound.

12. (canceled)

13. A method for the treatment and/or prophylaxis of angina pectoris, pulmonary hypertension, thromboembolic disorders or peripheral occlusive diseases comprising administering to a human or animal in need thereof using a pharmaceutically effective amount of at least one compound of claim 1.

14. The process of claim 6, wherein X¹ is halogen, and X² is selected from the group consisting of halogen and trifluoromethanesulfonyloxy.

15. A method for the treatment and/or prophylaxis of angina pectoris, pulmonary hypertension, thromboembolic disorders or peripheral occlusive diseases comprising administering to a human or animal in need thereof a pharmaceutically effective amount of a medicament as defined in claim 10.

16. A method for the treatment and/or prophylaxis of angina pectoris, pulmonary hypertension, thromboembolic disorders or peripheral occlusive diseases comprising administering to a human or animal in need thereof a pharmaceutically effective amount of a medicament as defined in claim 11.
